SEQUENCE LISTING (4 sequences)

SEQ ID NO: 1 - DNA, 28 bp, Artificial Sequence
  k-mer
ggtgttttttgtttagtattttttagag

SEQ ID NO: 2 - DNA, 25 bp, Artificial Sequence
  k-mer
agggtagatttgaggtagtttagga

SEQ ID NO: 3 - DNA, 25 bp, Artificial Sequence
  k-mer
taggtgttttttagggttgtttttt

SEQ ID NO: 4 - DNA, 24 bp, Artificial Sequence
  k-mer
gttggggaatttgttgtttattag

(19) United States
(12) Patent Application Publication
(10) Pub. No.: US 2020/0181703 A1
(43) Pub. Date: Jun. 11, 2020

(54) EPIGENOME-WIDE ASSOCIATION STUDY IDENTIFIES CARDIAC DEVELOPMENTAL GENE PATTERNING AND A NOVEL CLASS OF BIOMARKERS FOR HEART FAILURE

(71) Applicant: Siemens Healthcare GmbH, Erlangen (DE)

(72) Inventors: Andreas Emanuel POSCH, Wien (AT); Benjamin MEDER, Dossenheim (DE); Jan HAAS, Walldorf (DE); Hugo A. KATUS, Heidelberg (DE); Maximilian WUERSTLE, Baiersdorf (DE); Farbod SEDAGHAT-HAMEDANI, Heidelberg (DE); Andreas KELLER, St. Ingbert (DE); Cord Friedrich STAEHLER, Hirschberg An Der Bergstrasse (DE)

(73) Assignee: Siemens Healthcare GmbH, Erlangen (DE)

(21) Appl. No.: 16/315,129

(22) PCT Filed: Jul. 6, 2017
(86) PCT No.: PCT/EP2017/066941

§ 371 Date: Jan. 3, 2019

(30) Foreign Application Priority Data
Jul. 7, 2016 (EP) 16178413.7
Sep. 16, 2016 (EP) 16189099.1
May 16, 2017 (EP) 17171336.5

(51) Int. Cl. C12Q 1/6883; G16B 20/20; G16B 30/00; G16H 10/40; G16H 70/60; G16H 50/30

(52) U.S. Cl. CPC C12Q 1/6883; G16B 20/20; G16B 30/00; G16H 10/40; C12Q 2600/106; G16H 50/30; C12Q 2600/154; C12Q 2600/118; G16H 70/60

(57) ABSTRACT

The present invention relates to a method of determining markers for a disease from a patient, wherein information from epigenomics and/or the transcriptome from peripheral blood and a diseased tissue or information from epigenomics and the transcriptome from peripheral blood or a diseased tissue is used for obtaining the markers, as well as a method of determining a risk for a disease in a patient using the markers obtained thereby.

### PRIORITY STATEMENT

This application is the national phase under 35 U.S.C. § 371 of PCT International Application No. PCT/EP2017/066941 which as an International filing date of 6 Jul. 2017, which designated the United States of America and which claims priority to European Application No. EP 16178413.7 filed 6 Jul. 2016 and European Application No. EP 16189099.1 filed 16 Sep. 2016 and European Application No. 17171336.5 filed 16 May 2017. The entire contents of each application recited above is incorporated herein by reference.

### FIELD OF THE INVENTION

At least one embodiment of the invention generally relates to a method of determining markers for a disease from a patient, wherein information from epigenomics and/or the transcriptome from peripheral blood and a diseased tissue or information from epigenomics and the transcriptome from peripheral blood or a diseased tissue is used for obtaining the markers, as well as a method of determining a risk for a disease in a patient using the markers obtained thereby.

### BACKGROUND

The finding of markers for diagnosing diseases is a recently growing field due to new high-throughput methods of analysis of samples of patients as well as the availability of sufficient computing power to analyze the vast amount of data generated thereby.

This enables the identification of a variety of markers for a multitude of diseases, e.g. cardiac diseases, cancer, etc.

Heart failure (HF) is one major cause of morbidity and mortality in the general population and is the leading cause of hospitalization in individuals older than 65. Currently, 2% of general population suffers from HF, in elderly this increases to about 10%. In all western countries there is additionally an increasing prevalence of clinical manifest HF predicted.

HF is the result of an underlying cardiac disease. The two most common reasons for developing HF are systolic and/or diastolic dysfunction. For systolic HF, also referred to as HF-rEF the main reasons are ischemic heart disease due to coronary artery disease and myocardial infarction and non-ischemic causes such as Dilated Cardiomyopathy (DCM). DCM is a frequent heart muscle disease with an estimated prevalence of 1:2500 up to 1:500, which is caused by genetic mechanism, inflammation or infection. The progressive nature of the disorder is responsible for nearly 50,000 hospitalizations and 10,000 deaths per year in the US alone and is the main cause for heart transplantation in young adults. Overall, the incidence of the disease has continually increased over the past years and it was recognized that DCM has a substantial genetic contribution. It is estimated that about 30-40% of all DCM cases show familial aggregation and until now more than 40 different genes were found to cause genetic DCM.

Diagnosis and risk stratification of HF and DCM is still challenging and relies predominantly on symptoms, cardiovascular imaging parameters and biomarkers such as N-terminal pro b-type natriuretic peptide (Nt-ProBNP). Although highly accurate, Nt-ProBNP has its own caveats. For instance, several confounding factors can alter plasma level of Nt-ProBNP such as, age, gender, race, obesity, exercise, renal failure and anemia.

For better understanding of diseases like HF and to define therapy and diagnostic strategies, more accurate molecular biomarkers are needed. While several studies have now identified common genetic polymorphisms that are associated with DCM or heart failure—disclosed in Friedrichs, F. et al.: HBEGF, SRA1, and IK: Three cosegregating genes as determinants of cardiomyopathy, 395-403 (2009), doi:10.1101/gr.076653.108.19; and Villard, E. et al.: A genome-wide association study identifies two loci associated with heart failure due to dilated cardiomyopathy, Eur. Heart J. 32, 1065-76 (2011); epigenetic alterations—disclosed in Haas, J. et al.: Alterations in cardiac DNA methylation in human dilated cardiomyopathy, EMBO Mol. Med. 5, 413-429 (2013); or miRNA expression patterns, there still is an unmet need for reliable markers of HF/DCM, as well as other diseases.

Heart failure is the leading cause of hospitalization and death in Western countries. Over the last decades the genetic causes and molecular events driving the progression of heart failure have only been partially unravelled. Besides genetic predisposition (Meder B, et al., A genome-wide association study identifies 6p21 as novel risk locus for dilated cardiomyopathy. Eur Heart J. 2014; 35:1069-77; Villard E, et al., A genome-wide association study identifies two loci associated with heart failure due to dilated cardiomyopathy. Eur Heart J. 2011; 32:1065-76), it is long known that additional aspects including environmental factors and life-style influence the outbreak and course of myocardial failure (Hang C T, et al., Chromatin regulation by Brg1 underlies heart muscle development and disease. Nature. 2010; 466:62-7). The precise mode of action how such extrinsic, environmental factors may influence the phenotype of an individual and his disease is basically unknown.

Most recently, cardiovascular research has made first steps towards elucidating the role of the cardiac epigenome. During cardiac development, a series of dynamic changes in the methylation of gene bodies and Histone marks of developmental and sarcomeric genes were detected, a pattern that is partially re-established in failing cardiomyocytes (Hang C T, et al., Chromatin regulation by Brg1 underlies heart muscle development and disease. Nature. 2010; 466:62-7; Sergeeva I A, et al., Identification of a regulatory domain controlling the Nppa-Nppb gene cluster during heart development and stress. Development. 2016; 143:2135-46; Greco C M, et al., DNA hydroxymethylation controls cardiomyocyte gene expression in development and hypertrophy. Nature communications. 2016; 7:12418). In the adaption to stress and during hypertrophy, similar observations were made in engineered heart tissue from rats, pointing towards conservation of methylation-based gene patterning across species (Stenzig J, et al., DNA methylation in an engineered heart tissue model of cardiac hypertrophy: common signatures and effects of DNA methylation inhibitors. Basic Res Cardiol. 2016; 111:9). While these studies indicate a potentially central role of epigenetic regulation in the heart and highly sophisticated technologies exist to assess Histone-modifications or DNA methylation at a base-pair resolution, the lack of availability of myocardial specimen from patients is a major roadblock for elucidating the impact of such changes on complex cardiovascular traits (Greco C M and Condorelli G. Epigenetic modifications and noncoding RNAs in cardiac hypertrophy and failure. Nat Rev Cardiol. 2015; 12:488-97). Hence, mainly animal studies or investigations of very small clinical cohorts could shed some light onto the presence and role of chemical alterations of cardiac DNA in heart failure or cardiomyopathy.

One of the pioneering studies on DNA methylation in heart failure was published by the group of Roger Foo in 2011 (Movassagh M, et al., Distinct epigenomic features in endstage failing human hearts. Circulation. 2011; 124:2411-22). They identified that epigenetic changes in heart failure occur not uniformly across the genome, but are concentrated in promoter CpG islands, intragenic CpG islands and gene bodies. The limitation of this study was the very small sample size of only 4 end-stage heart failure cardiac explants that were investigated. In 2013 Haas et al. were able to identify and replicate genome-wide signatures of lower resolution DNA methylation changes in living patients suffering from Dilated Cardiomyopathy (DCM), which is a major cause of non-ischemic heart failure (Haas J, et al., Alterations in cardiac DNA methylation in human dilated cardiomyopathy. EMBO Mol Med. 2013; 5:413-29). In this study, they identified a set of novel candidate genes that are potentially involved in heart failure, such as ADORA2A and LY75. Another of the few available examples identified Methyl-CpG-binding protein 2 (MeCP2), a downstream effector of DNA methylation, to be repressed during heart failure in humans and reactivated after mechanical unloading of the left ventricle by assist devices (Mayer S C, et al., Adrenergic Repression of the Epigenetic Reader MeCP2 Facilitates Cardiac Adaptation in Chronic Heart Failure. Circ. Res. 2015; 117:622-33), pointing towards a potential role of targeted epigenetic therapies for heart failure.

Biochemical DNA modification resembles a crucial regulatory layer between genetic information, environmental factors and the transcriptome.

### SUMMARY

To identify epigenetic susceptibility regions and novel biomarkers linked to myocardial dysfunction and heart failure, the inventors performed the first multi-omics study in myocardial tissue and blood of patients with Dilated Cardiomyopathy (DCM) and controls.

The present inventors dissected for the first time high-resolution epigenome-wide cardiac and blood DNA methylation in conjunction with mRNA and whole-genome sequencing in a large cohort of densely-phenotyped patients with systolic heart failure due to DCM. They provide the yet largest dataset of cardiac and blood DNA methylation profiles and identified key epigenomic patterns that are distinct fingerprints of human heart failure.

The present inventors have found that improved marker finding is possible when more than one characteristic of the sample, e.g. the nucleic acid sequence, is considered. Further, it was found that also improved marker finding is possible when more than one sample from different sources is considered, wherein one if preferably from tissue related to a disease and a further one from peripheral blood.

In a first aspect, the present invention is related to a method of determining markers for a disease from a patient, comprising

obtaining or providing at least one sample of peripheral blood and at least one sample of a diseased tissue of the patient diagnosed with the disease;

obtain an epigenomics profile and/or analyze a transcriptome of the at least one sample of the peripheral blood and the at least one sample of the diseased tissue;

compare the epigenomics profile and/or the transcriptome to an epigenomics profile and/or a transcriptome of a suitable control, respectively; and

determine one or more alteration in the epigenomics profile and/or the transcriptome in both the at least one sample of the peripheral blood and at least one sample of the diseased tissue of the patient diagnosed with the disease.

Further, the present invention relates to a method of determining markers for a disease from a patient, comprising

obtaining or providing at least one sample of peripheral blood or at least one sample of a diseased tissue of the patient diagnosed with the disease;

obtain an epigenomics profile and analyze a transcriptome of the at least one sample of the peripheral blood or the at least one sample of the diseased tissue;

compare the epigenomics profile and the transcriptome to an epigenomics profile and a transcriptome of a suitable control, respectively; and

determine one or more alteration in the epigenomics profile and the transcriptome in either the at least one sample of the peripheral blood or the at least one sample of the diseased tissue of the patient diagnosed with the disease.

Additionally, a method of determining a risk for a disease in a patient, comprising

obtaining or providing an epigenomics profile and/or a transcriptome of at least one sample of the peripheral blood and/or the a diseased tissue, e.g. the myocard/myocardium, of the patient, and

determining the presence of at least one marker as determined by the method of the first or second aspect is disclosed.

Further disclosed is a data bank comprising specific markers for heart failure and/or dilated cardiomyopathy in a patient, the use of this databank in a method of determining a risk for heart failure and/or dilated cardiomyopathy in a patient, and the use of the specific markers as a marker for heart failure and/or dilated cardiomyopathy in a patient.

In addition, a method of determining a risk for a disease in a patient, comprising

obtaining or providing data of an epigenomics profile and/or a transcriptome of at least one sample of the peripheral blood and/or a diseased tissue of the patient, and

determining the presence of at least one marker as determined by the method of the first or second aspect is disclosed, as well as a computer program product comprising computer executable instructions which, when executed, perform such a method.

Further aspects and embodiments of the invention are disclosed in the dependent claims and can be taken from the following description, figures and examples, without being limited thereto.

### BRIEF DESCRIPTION OF THE DRAWINGS

The enclosed drawings should illustrate embodiments of the present invention and convey a further understanding thereof. In connection with the description they serve as explanation of concepts and principles of the invention. Other embodiments and many of the stated advantages can be derived in relation to the drawings. The elements of the drawings are not necessarily to scale towards each other. Identical, functionally equivalent and acting equal features and components are denoted in the figures of the drawings with the same reference numbers, unless noted otherwise.

FIGS. 1 to 3 show schematically concepts for finding markers for a disease according to a method of the present invention.

FIG. 4 shows the relation between Simes significance level (SL) for association between DNA methylation and gene expression at increasing distances (D) as determined in the present Example 1.

FIGS. 5 to 21 show data referred to and obtained in present Example 2.

### DETAILED DESCRIPTION OF EXAMPLE EMBODIMENTS

### Definitions

Unless defined otherwise, technical and scientific terms used herein have the same meaning as commonly understood by one of ordinary skill in the art to which this invention belongs.

The term “nucleic acid molecule” refers to a polynucleotide molecule having a defined sequence. It comprises DNA molecules, RNA molecules, nucleotide analog molecules and combinations and derivatives thereof, such as DNA molecules or RNA molecules with incorporated nucleotide analogs or cDNA.

The term “nucleic acid sequence information” relates to information which can be derived from the sequence of a nucleic acid molecule, such as the sequence itself or a variation in the sequence as compared to a reference sequence.

The term “mutation” relates to a variation in the sequence as compared to a reference sequence. A mutation is for example a deletion of one or multiple nucleotides, an insertion of one or multiple nucleotides, or substitution of one or multiple nucleotides, duplication of one or a sequence of multiple nucleotides, translocation of one or a sequence of multiple nucleotides, and, in particular, a single nucleotide polymorphism (SNP).

In the context of the present invention a “sample” is a sample which comprises at least epigenetic information and/or information regarding the transcriptome of a patient. Examples for samples are: cells, tissue, biopsy specimens, body fluids, blood, urine, saliva, sputum, plasma, serum, cell culture supernatant, swab sample and others.

An epigenomics profile corresponds to the multitude of all epigenomic modifications, i.e. DNA methylation, Histone methylation, etc., that can occur in a patient.

A transcriptomics profile corresponds to the multitude of all transcribed nucleic acids, i.e. messenger RNA, micro RNAs, non-coding RNAs, etc.

Peripheral blood refers to the circulating pool of blood within the patient.

According to certain embodiments, the patient in the present methods is a vertebrate, more preferably a mammal and most preferred a human patient.

A vertebrate within the present invention refers to animals having a vertebrae, which includes mammals—including humans, birds, reptiles, amphibians and fishes. The present invention thus is not only suitable for human medicine, but also for veterinary medicine.

New and highly efficient methods of sequencing nucleic acids referred to as next generation sequencing have opened the possibility of large scale genomic analysis. The term “next generation sequencing” or “high throughput sequencing” refers to high-throughput sequencing technologies that parallelize the sequencing process, producing thousands or millions of sequences at once. Examples include Massively Parallel Signature Sequencing (MPSS), Polony sequencing, 454 pyrosequencing, Illumina (Solexa) sequencing, SOLiD sequencing, Ion semiconductor sequencing, DNA nanoball sequencing, Helioscope™ single molecule sequencing, Single Molecule SMRT™ sequencing, Single Molecule real time (RNAP) sequencing, Nanopore DNA sequencing, Sequencing By Hybridization, Amplicon Sequencing, GnuBio.

Before the invention is described in exemplary detail, it is to be understood that this invention is not limited to the particular component parts of the process steps of the methods described herein as such methods may vary. It is also to be understood that the terminology used herein is for purposes of describing particular embodiments only, and is not intended to be limiting. It must be noted that, as used in the specification and the appended claims, the singular forms “a,” “an” and “the” include singular and/or plural referents unless the context clearly dictates otherwise. For example, the term “a” as used herein can be understood as one single entity or in the meaning of “one or more” entities. It is also to be understood that plural forms include singular and/or plural referents unless the context clearly dictates otherwise. It is moreover to be understood that, in case parameter ranges are given which are delimited by numeric values, the ranges are deemed to include these limitation values.

In a first aspect, the present invention relates to a method of determining markers for a disease from a patient, comprising

obtaining or providing at least one sample of peripheral blood and at least one sample of a diseased tissue of the patient diagnosed with the disease;

obtain an epigenomics profile and/or analyze a transcriptome of the at least one sample of the peripheral blood and the at least one sample of the diseased tissue;

compare the epigenomics profile and/or the transcriptome to an epigenomics profile and/or a transcriptome of a suitable control, respectively; and

determine one or more alteration in the epigenomics profile and/or the transcriptome in both the at least one sample of the peripheral blood and at least one sample of the diseased tissue of the patient diagnosed with the disease.

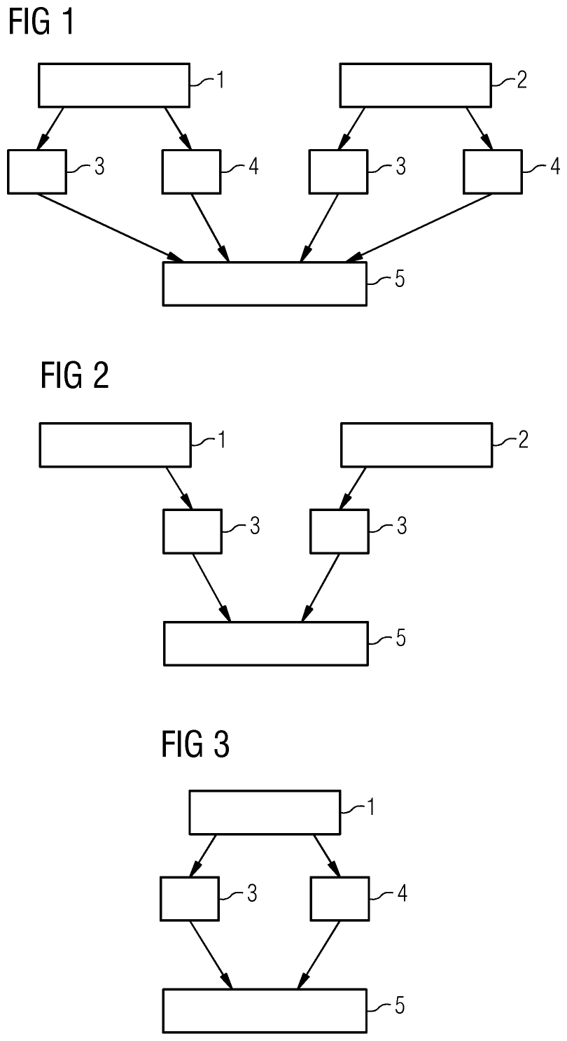

In this first aspect, thus at least two different samples are obtained, and these can be analyzed with regard to the epigenomics profile, the transcriptome, or both. This is schematically shown in exemplary FIGS. 1 and 2.

According to FIG. 1, two samples are provided, e.g. from a human, i.e. one sample from a diseased tissue 1, e.g. the myocard, and one sample from peripheral blood 2. For both samples the epigenomics profile 3 and the transcriptome 4 are obtained and analyzed with the present method, to obtain one or more markers 5. As an alternative, only the epigenomics profile 3 or the transcriptome 4 can be obtained and analyzed when two samples are provided (not shown). Preferably, only either the epigenomics profile 3 or the transcriptome 4 are then analyzed from both samples in such a case, i.e. not the epigenomics profile 3 from one sample and the transcriptome 4 from the other sample.

In an alternative method shown in FIG. 2, again two samples are provided, e.g. from a human, i.e. one sample from a diseased tissue 1, e.g. the myocard, and one sample from peripheral blood 2. For both samples only the epigenomics profile 3 is obtained, though, and analyzed with the present method, to obtain one or more markers 5. Of course, it is also possible to analyze the transcriptome 4 only instead of the epigenomics profile 3 in the scheme shown in FIG. 2.

In a second aspect, the present invention relates to a method of determining markers for a disease from a patient, comprising

obtaining or providing at least one sample of peripheral blood or at least one sample of a diseased tissue of the patient diagnosed with the disease;

obtain an epigenomics profile and analyze a transcriptome of the at least one sample of the peripheral blood or the at least one sample of the diseased tissue;

compare the epigenomics profile and the transcriptome to an epigenomics profile and a transcriptome of a suitable control, respectively; and

determine one or more alteration in the epigenomics profile and the transcriptome in either at least one sample of the peripheral blood or the at least one sample of the diseased tissue of the patient diagnosed with the disease.

In this second aspect, thus at least one sample is obtained, but not from different sources. This sample is then analyzed with regard to both the epigenomics profile and the transcriptome. This is schematically shown in exemplary FIG. 3.

According to FIG. 3, one sample is provided, e.g. from a human, i.e. one sample from a diseased tissue 1, e.g. the myocard. For this sample both the epigenomics profile 3 and the transcriptome 4 are obtained and analyzed with the present method, to obtain one or more markers 5. Of course, it is also possible to provide one sample from the peripheral blood 2 instead of from the diseased tissue 1 in this method, though.

The disease in the present invention is not particularly limited. According to certain embodiments, it is a non-infectious disease, particularly a cardiovascular disease. According to certain embodiments, the disease is heart failure (HF) and/or dilated cardiomyopathy (DCM). In such a case, the sample of the diseased tissue can be obtained from myocardial tissue.

The obtaining of the sample is also not particularly limited, but is preferably non-invasive, e.g. is taken from a stock or from a storage, etc.

Further, also the obtaining of the epigenomics profile as well as the analysis of the transcriptome are not particularly limited and can be suitably carried out using known means, including sequencing, bead array or microarray technology.

Also, the comparison to an epigenomics profile and/or a transcriptome of a suitable control is not particularly limited and can be done in any way, e.g. using computational programs, etc. Further, the alteration in the epigenomics profile and/or the transcriptome is not particularly limited. According to certain embodiments, the alteration is a hyper and/or hypo methylation and/or a change in chromatin marks and/or a change in the RNA (e.g. messenger RNA, micro RNA, non-coding RNA etc.) expression level, e.g. an increase or decrease in RNA expression level, wherein all combinations are possible, e.g. a hyper methylation in combination with a decrease or an increase in RNA expression level, or a hypo methylation in combination with a decrease or an increase in RNA expression level.

The control is not limited as well and can be suitably chosen based on the patient. For example, a control can be obtained from one or more patients not diagnosed with the disease, or from a publicly known control that is not affected by the disease. According to certain embodiments, the one or more alteration is determined with regard to the nucleic acid sequence information of the patient, e.g. the genome. According to certain embodiments, the patient is a human. According to certain embodiments, the patient is a human and the control is reference genome hg19, as provided by e.g. Genome Reference Consortium and the University of California, Santa Cruz (GRCh37/hg19, downloadable from http://hgdownload.cse.ucsc.edu/goldenPath/hg19/bigZips/ and http://www.ncbi.nlm.nih.gov/projects/genome/assembly/grc/human/). Gene regions are based on the GRCh37/hg19 and the Gencode 19 gene model (http://www.gencodegenes.org/).

According to certain embodiments a plurality of samples of the peripheral blood and/or the diseased tissue are obtained or provided from patients diagnosed with the disease. This way statistical significance of the found markers can be improved.

In a further aspect, the present invention relates to a method of determining a risk for a disease in a patient, comprising

obtaining or providing an epigenomics profile and/or a transcriptome of at least one sample of the peripheral blood and/or a diseased tissue, e.g. the myocard, of the patient, and

determining the presence of at least one marker as determined by the method of the first or the second aspect.

Again, the obtaining of the sample is not particularly limited, but is preferably non-invasive, e.g. is taken from a stock or from a storage, etc.

According to certain embodiments, the diseased tissue is the myocard, and preferably the disease is heart failure and/or dilated cardiomyopathy.

For heart failure and/or dilated cardiomyopathy, a list of markers for improved determination of a risk for these diseases has been found by the present methods of the first and second aspect. These are shown in the following tables.

Thus, according to certain embodiments, the at least one epigenetic and/or transcriptomic marker for determining a risk for heart failure and/or dilated cardiomyopathy

is contained in genomic regions with regard to reference genome hg19 that show coordinated hyper/hypo methylation in HF/DCM in peripheral blood and myocardial tissue and are associated with RNA expression levels and is chosen from the sequences disclosed in Table 1, preferably Table 1a, particularly preferably Table 1b; and/or

is contained in genomic regions with regard to reference genome hg19 that show hyper/hypo methylation in HF/DCM in myocardial tissue and are associated with RNA expression levels and is chosen from the sequences disclosed in Table 2, preferably Table 2a, particularly preferably Table 2b; and/or

is contained in genomic regions with regard to reference genome hg19 that show coordinated hyper/hypo methylation in HF/DCM in peripheral blood and myocardial tissue and is chosen from the sequences disclosed in Table 3, preferably Table 3a, particularly preferably Table 3b; and/or

is contained in genomic regions with regard to reference genome hg19 that show dysmethylation in HF/DCM in peripheral blood and is chosen from the sequences disclosed in Table 4; and/or

is contained in genomic regions with regard to the reference Infinium HumanMethylation450K database and the reference genome hg19, respectively, that show dysmethylation in HF/DCM in peripheral blood and is chosen from the cpg IDs or positions disclosed in Table 5; and/or

is contained in genomic regions with regard to reference genome hg19 that show dysmethylation in HF/DCM in peripheral blood and is chosen from the sequences disclosed in Table 6; and/or

is contained in genomic regions with regard to the reference Infinium HumanMethylation450K database and the reference genome hg19, respectively, that show dysmethylation in HF/DCM in peripheral blood and is chosen from the cpg IDs or positions disclosed in Table 7; and/or

is contained in genomic regions with regard to reference genome hg19 that show dysmethylation in HF/DCM in peripheral blood and is chosen from the sequences disclosed in Table 8; and/or

is contained in genomic regions with regard to the reference Infinium HumanMethylation450K database and the reference genome hg19, respectively, that show dysmethylation in HF/DCM in peripheral blood and is chosen from the cpg IDs or positions disclosed in Table 9; and/or

is contained in genomic regions with regard to reference genome hg19 that show dysmethylation in HF/DCM in peripheral blood and myocardial tissue and are associated with RNA expression levels and is chosen from the ANF and/or BNP loci and/or the sequences disclosed in Table 10. In the tables 1, 1a, 1b, 2, 2a, 2b, 3, 3a, 3b, 4, 6, 8, and 10 the sequences are the nucleic acid sequences between the positions in the columns titled start and end in the respective chromosomes (chr.), including the positions given under start and end, with regard to reference genome hg19. Further, in Tables 1, 1a, 1b, 2, 2a, 2b, 3, 3a, and 3b sequences are given in columns 1 and 2 as well as in columns 4 and 5 for brevity sake, i.e. one sequence is between and including the positions in columns 1 and 2, and one sequence is between and including the positions in columns 4 and 5. Tables 5, 7 and 9 represent distinct cpg IDs with regard to the reference Infinium HumanMethylation450K database and positions with regard to reference genome hg19 that show statistically significant dysmethylation in peripheral blood.

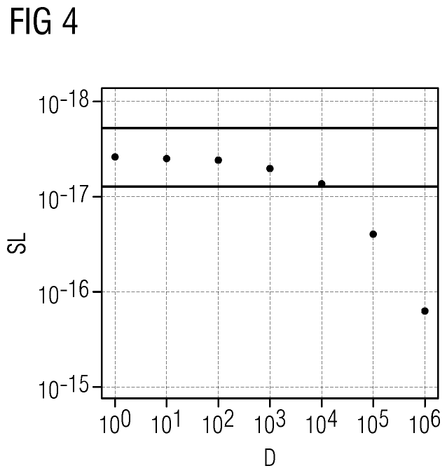

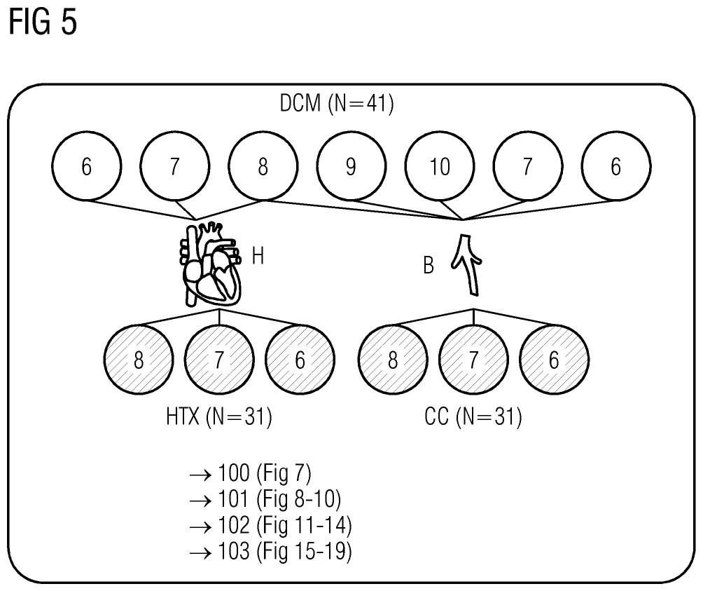

The inventors have found that a hyper/hypo methylation can affect both strands and therefore genes on both strands. They further found out that it also does not only affect the gene regions itself, but also the surrounding area, particularly within a region of 10000 base pairs, more particularly within a region of 1000 base pairs. Not only coding regions may be influenced thereby, but also regions surrounding the coding regions, e.g. promoter regions, etc. Thus, while the most significant results may be found in only a very limited region, hyper/hypo methylation was observed within a broad region around the position, without a significant change in the significance within 10000 base pairs, as is also shown in e.g. FIG. 4. Tables 1, 2, 3, 4, 6, 8, and 10 thus represent the respective ranges for a gene range −10000 base pairs at the start and +10000 at the end for genes affected by a change in methylation, i.e. a hyper/hypo methylation, whereas tables 1a, 2a and 3a represent the sequence ranges for the affected gene, and tables 1b, 2b and 3b represent the most significant methylation alterations.

TABLE 1
Markers, given as nucleic acid sequence with start and end,
that show coordinated hyper/hypo methylation in HF/DCM in peripheral
blood and myocardial tissue and are associated with RNA expression
levels (with regard to reference genome hg19)

| start | end | chr. | start | end | chr. |
|---|---|---|---|---|---|
| 56398246 | 56419869 | 17 | 129695326 | 129894119 | 10 |
| 56392812 | 56503127 | 17 | 14762811 | 14800933 | 2 |
| 77275701 | 77339673 | 15 | 407934 | 452011 | 11 |
| 82650409 | 83840204 | 16 | 131230374 | 132216716 | 11 |
| 79402358 | 80885905 | 2 | 19230868 | 19291495 | 11 |
| 80505484 | 80541874 | 2 | 150989427 | 151188609 | 4 |
| 217487552 | 217539159 | 2 | | | |

TABLE 1a
Preferred markers, given as nucleic acid sequence with start and
end, that show coordinated hyper/hypo methylation in HF/DCM in
peripheral blood and myocardial tissue and are associated with
RNA expression levels (with regard to reference genome hg19)

| start | end | chr. | start | end | chr. |
|---|---|---|---|---|---|
| 56408246 | 56409869 | 17 | 129705326 | 129884119 | 10 |
| 56402812 | 56493127 | 17 | 14772811 | 14790933 | 2 |
| 77285701 | 77329673 | 15 | 417934 | 442011 | 11 |
| 82660409 | 83830204 | 16 | 131240374 | 132206716 | 11 |
| 79412358 | 80875905 | 2 | 19240868 | 19281495 | 11 |
| 80515484 | 80531874 | 2 | 150999427 | 151178609 | 4 |
| 217497552 | 217529159 | 2 | | | |

TABLE 1b
Particularly preferred markers, given as nucleic acid sequence with
start and end, that show coordinated hyper/hypo methylation in HF/DCM
in peripheral blood and myocardial tissue and are associated with
RNA expression levels (with regard to reference genome hg19)

| start | end | chr. | start | end | chr. |
|---|---|---|---|---|---|
| 56408197 | 56408198 | 17 | 129846082 | 129846083 | 10 |
| 56408197 | 56408198 | 17 | 14772731 | 14772732 | 2 |
| 77287656 | 77287657 | 15 | 430036 | 430037 | 11 |
| 82970452 | 82970453 | 16 | 131533284 | 131533285 | 11 |
| 80531656 | 80531657 | 2 | 19250190 | 19250191 | 11 |
| 80531656 | 80531657 | 2 | 151038391 | 151038392 | 4 |
| 217508851 | 217508852 | 2 | | | |

TABLE 2
Markers, given as nucleic acid sequence with start and
end, that show hyper/hypo methylation in HF/DCM in
myocardial tissue and are associated with RNA expression
levels (with regard to reference genome hg19)

| start | end | chr. | start | end | chr. |
|---|---|---|---|---|---|
| 119415670 | 119542179 | 1 | 3117166 | 3150543 | 20 |
| 208185588 | 208427665 | 1 | 52173605 | 52236446 | 20 |
| 114835997 | 114860636 | 12 | 36150099 | 37386965 | 21 |
| 114781737 | 114856247 | 12 | 20773529 | 20860170 | 22 |
| 74954874 | 75089306 | 14 | 38854068 | 38889452 | 22 |
| 222272748 | 222448922 | 2 | 123318897 | 123613178 | 3 |
| 11895767 | 11918402 | 1 | 127397910 | 127552051 | 3 |
| 151013448 | 151052801 | 1 | 15481641 | 15573258 | 3 |
| 154117785 | 154177124 | 1 | 185813458 | 186090026 | 3 |
| 16320732 | 16345302 | 1 | 42685177 | 42719072 | 3 |
| 183888797 | 184016863 | 1 | 43318005 | 43476256 | 3 |
| 27658514 | 27690421 | 1 | 56751447 | 57123357 | 3 |
| 53961911 | 54209877 | 1 | 146668780 | 146869787 | 4 |
| 842246 | 866396 | 1 | 15331443 | 15457790 | 4 |
| 125455724 | 125709783 | 10 | 186275033 | 186327053 | 4 |
| 50212291 | 50333554 | 10 | 54315469 | 54577572 | 4 |
| 71019741 | 71171638 | 10 | 76944836 | 76972568 | 4 |
| 72962328 | 73072621 | 10 | 138717636 | 138740885 | 5 |
| 90629492 | 90744910 | 10 | 168078746 | 168738133 | 5 |
| 10584639 | 10725535 | 11 | 58254866 | 59827947 | 5 |
| 33870123 | 33923836 | 11 | 71393062 | 71515395 | 5 |
| 65647876 | 65669105 | 11 | 33229788 | 33254287 | 6 |
| 68070078 | 68226743 | 11 | 33530330 | 33558019 | 6 |
| 73009335 | 73090136 | 11 | 106495724 | 106557590 | 7 |
| 73101533 | 73319234 | 11 | 149554787 | 149587699 | 7 |
| 93852095 | 93925138 | 11 | 149560058 | 149587784 | 7 |
| 94429598 | 94619918 | 11 | 47304753 | 47632156 | 7 |
| 95699763 | 96086344 | 11 | 756339 | 839190 | 7 |
| 26101963 | 26242825 | 12 | 128796780 | 129123499 | 8 |
| 102094967 | 102385456 | 13 | 25689247 | 25912913 | 8 |
| 108860728 | 108896603 | 13 | 116197012 | 116370018 | 9 |
| 53181606 | 53227919 | 13 | 9701791 | 9799172 | 1 |
| 96495662 | 96570417 | 14 | 28189056 | 28223196 | 1 |
| 101830819 | 102075405 | 15 | 198597802 | 198736545 | 1 |
| 68584051 | 68734501 | 15 | 68582306 | 68634585 | 2 |
| 74456013 | 74479213 | 15 | 235391686 | 235415697 | 2 |
| 83766160 | 83823606 | 15 | 47366412 | 47410127 | 11 |
| 15787030 | 15960890 | 16 | 63964151 | 64001354 | 11 |
| 27788851 | 28084830 | 16 | 46690056 | 46796006 | 13 |
| 31119400 | 31140068 | 16 | 89169385 | 89209714 | 15 |
| 49301829 | 49325742 | 16 | 27314990 | 27386099 | 16 |
| 17736829 | 17885736 | 17 | 30184149 | 30210397 | 16 |
| 42102004 | 42154987 | 17 | 31261312 | 31354213 | 16 |
| 5009734 | 5088329 | 17 | 84589201 | 84661683 | 16 |
| 62214588 | 62350661 | 17 | 85922410 | 85966215 | 16 |
| 78183499 | 78237299 | 17 | 7229849 | 7264797 | 17 |
| 78992934 | 79018501 | 17 | 76116852 | 76149049 | 17 |
| 8367524 | 8544079 | 17 | 10371512 | 10407291 | 19 |
| 31755852 | 31850453 | 19 | 36385304 | 36409197 | 19 |
| 7102267 | 7304045 | 19 | 51864861 | 51885969 | 19 |
| 176991341 | 177047830 | 2 | 39304489 | 39327880 | 20 |
| 223054608 | 223173715 | 2 | 46295869 | 46361904 | 21 |
| 23598089 | 23941481 | 2 | 44558837 | 44625413 | 22 |
| 55189326 | 55349757 | 2 | | | |

TABLE 2a
Preferred markers, given as nucleic acid sequence with
start and end, that show hyper/hypo methylation in HF/DCM
in myocardial tissue and are associated with RNA expression
levels (with regard to reference genome hg19)

| start | end | chr. | start | end | chr. |
|---|---|---|---|---|---|
| 119425670 | 119532179 | 1 | 3127166 | 3140543 | 20 |
| 208195588 | 208417665 | 1 | 52183605 | 52226446 | 20 |
| 114845997 | 114850636 | 12 | 36160099 | 37376965 | 21 |
| 114791737 | 114846247 | 12 | 20783529 | 20850170 | 22 |
| 74964874 | 75079306 | 14 | 38864068 | 38879452 | 22 |
| 222282748 | 222438922 | 2 | 123328897 | 123603178 | 3 |
| 11905767 | 11908402 | 1 | 127407910 | 127542051 | 3 |
| 151023448 | 151042801 | 1 | 15491641 | 15563258 | 3 |
| 154127785 | 154167124 | 1 | 185823458 | 186080026 | 3 |
| 16330732 | 16335302 | 1 | 42695177 | 42709072 | 3 |
| 183898797 | 184006863 | 1 | 43328005 | 43466256 | 3 |
| 27668514 | 27680421 | 1 | 56761447 | 57113357 | 3 |
| 53971911 | 54199877 | 1 | 146678780 | 146859787 | 4 |
| 852246 | 856396 | 1 | 15341443 | 15447790 | 4 |
| 125465724 | 125699783 | 10 | 186285033 | 186317053 | 4 |
| 50222291 | 50323554 | 10 | 54325469 | 54567572 | 4 |
| 71029741 | 71161638 | 10 | 76954836 | 76962568 | 4 |
| 72972328 | 73062621 | 10 | 138727636 | 138730885 | 5 |
| 90639492 | 90734910 | 10 | 168088746 | 168728133 | 5 |
| 10594639 | 10715535 | 11 | 58264866 | 59817947 | 5 |
| 33880123 | 33913836 | 11 | 71403062 | 71505395 | 5 |
| 65657876 | 65659105 | 11 | 33239788 | 33244287 | 6 |
| 68080078 | 68216743 | 11 | 33540330 | 33548019 | 6 |
| 73019335 | 73080136 | 11 | 106505724 | 106547590 | 7 |
| 73111533 | 73309234 | 11 | 149564787 | 149577699 | 7 |
| 93862095 | 93915138 | 11 | 149570058 | 149577784 | 7 |
| 94439598 | 94609918 | 11 | 47314753 | 47622156 | 7 |
| 95709763 | 96076344 | 11 | 766339 | 829190 | 7 |
| 26111963 | 26232825 | 12 | 128806780 | 129113499 | 8 |
| 102104967 | 102375456 | 13 | 25699247 | 25902913 | 8 |
| 108870728 | 108886603 | 13 | 116207012 | 116360018 | 9 |
| 53191606 | 53217919 | 13 | 9711791 | 9789172 | 1 |
| 96505662 | 96560417 | 14 | 28199056 | 28213196 | 1 |
| 101840819 | 102065405 | 15 | 198607802 | 198726545 | 1 |
| 68594051 | 68724501 | 15 | 68592306 | 68624585 | 2 |
| 74466013 | 74469213 | 15 | 235401686 | 235405697 | 2 |
| 83776160 | 83813606 | 15 | 47376412 | 47400127 | 11 |
| 15797030 | 15950890 | 16 | 63974151 | 63991354 | 11 |
| 27798851 | 28074830 | 16 | 46700056 | 46786006 | 13 |
| 31129400 | 31130068 | 16 | 89179385 | 89199714 | 15 |
| 49311829 | 49315742 | 16 | 27324990 | 27376099 | 16 |
| 17746829 | 17875736 | 17 | 30194149 | 30200397 | 16 |
| 42112004 | 42144987 | 17 | 31271312 | 31344213 | 16 |
| 5019734 | 5078329 | 17 | 84599201 | 84651683 | 16 |
| 62224588 | 62340661 | 17 | 85932410 | 85956215 | 16 |
| 78193499 | 78227299 | 17 | 7239849 | 7254797 | 17 |
| 79002934 | 79008501 | 17 | 76126852 | 76139049 | 17 |
| 8377524 | 8534079 | 17 | 10381512 | 10397291 | 19 |
| 31765852 | 31840453 | 19 | 36395304 | 36399197 | 19 |
| 7112267 | 7294045 | 19 | 51874861 | 51875969 | 19 |
| 177001341 | 177037830 | 2 | 39314489 | 39317880 | 20 |
| 223064608 | 223163715 | 2 | 46305869 | 46351904 | 21 |
| 23608089 | 23931481 | 2 | 44568837 | 44615413 | 22 |
| 55199326 | 55339757 | 2 | | | |

TABLE 2b
Particularly preferred markers, given as nucleic acid sequence
with start and end, that show hyper/hypo methylation in
HF/DCM in myocardial tissue and are associated with RNA
expression levels (with regard to reference genome hg19)

| start | end | chr. | start | end | chr. |
|---|---|---|---|---|---|
| 119526255 | 119526256 | 1 | 78190755 | 78190756 | 17 |
| 119526882 | 119526883 | 1 | 79012396 | 79012397 | 17 |
| 119527008 | 119527009 | 1 | 8382941 | 8382942 | 17 |
| 119527111 | 119527112 | 1 | 31848310 | 31848311 | 19 |
| 119532189 | 119532190 | 1 | 7224513 | 7224514 | 19 |
| 119532542 | 119532543 | 1 | 7224713 | 7224714 | 19 |
| 119534644 | 119534645 | 1 | 177025198 | 177025199 | 2 |
| 208293478 | 208293479 | 1 | 223164925 | 223164926 | 2 |
| 208405868 | 208405869 | 1 | 23843711 | 23843712 | 2 |
| 208412585 | 208412586 | 1 | 55339939 | 55339940 | 2 |
| 114841202 | 114841203 | 12 | 3148787 | 3148788 | 20 |
| 114841671 | 114841672 | 12 | 52199729 | 52199730 | 20 |
| 114841708 | 114841709 | 12 | 52199748 | 52199749 | 20 |
| 114841792 | 114841793 | 12 | 36577638 | 36577639 | 21 |
| 114845868 | 114845869 | 12 | 20780298 | 20780299 | 22 |
| 114846162 | 114846163 | 12 | 38864868 | 38864869 | 22 |
| 114846162 | 114846163 | 12 | 123372199 | 123372200 | 3 |
| 114846321 | 114846322 | 12 | 127494852 | 127494853 | 3 |
| 114846321 | 114846322 | 12 | 15540137 | 15540138 | 3 |
| 114846399 | 114846400 | 12 | 186080868 | 186080869 | 3 |
| 114846399 | 114846400 | 12 | 42694144 | 42694145 | 3 |
| 114846412 | 114846413 | 12 | 42694803 | 42694804 | 3 |
| 75043777 | 75043778 | 14 | 43405624 | 43405625 | 3 |
| 75072120 | 75072121 | 14 | 56789178 | 56789179 | 3 |
| 75086513 | 75086514 | 14 | 146740968 | 146740969 | 4 |
| 222323493 | 222323494 | 2 | 146841472 | 146841473 | 4 |
| 222333289 | 222333290 | 2 | 15397288 | 15397289 | 4 |
| 222367110 | 222367111 | 2 | 186283800 | 186283801 | 4 |
| 11900652 | 11900653 | 1 | 54357316 | 54357317 | 4 |
| 151021364 | 151021365 | 1 | 76945459 | 76945460 | 4 |
| 154164699 | 154164700 | 1 | 138718914 | 138718915 | 5 |
| 16335452 | 16335453 | 1 | 168139607 | 168139608 | 5 |
| 184005063 | 184005064 | 1 | 58882753 | 58882754 | 5 |
| 27677240 | 27677241 | 1 | 71402031 | 71402032 | 5 |
| 54058616 | 54058617 | 1 | 33240333 | 33240334 | 6 |
| 854824 | 854825 | 1 | 33551533 | 33551534 | 6 |
| 125618188 | 125618189 | 10 | 106507474 | 106507475 | 7 |
| 50289110 | 50289111 | 10 | 149578384 | 149578385 | 7 |
| 50298306 | 50298307 | 10 | 149578384 | 149578385 | 7 |
| 71094286 | 71094287 | 10 | 47479433 | 47479434 | 7 |
| 73026288 | 73026289 | 10 | 811491 | 811492 | 7 |
| 90712739 | 90712740 | 10 | 128808063 | 128808064 | 8 |
| 10716164 | 10716165 | 11 | 25908057 | 25908058 | 8 |
| 33913716 | 33913717 | 11 | 25908279 | 25908280 | 8 |
| 65659393 | 65659394 | 11 | 25908503 | 25908504 | 8 |
| 68142234 | 68142235 | 11 | 116359818 | 116359819 | 9 |
| 73034459 | 73034460 | 11 | 9711791 | 9789172 | 1 |
| 73108402 | 73108403 | 11 | 28199056 | 28213196 | 1 |
| 93885254 | 93885255 | 11 | 198607802 | 198726545 | 1 |
| 94521117 | 94521118 | 11 | 68592306 | 68624585 | 2 |
| 96071506 | 96071507 | 11 | 235401686 | 235405697 | 2 |
| 26111821 | 26111822 | 12 | 47376412 | 47400127 | 11 |
| 102104991 | 102104992 | 13 | 63974151 | 63991354 | 11 |
| 108867111 | 108867112 | 13 | 46700056 | 46786006 | 13 |
| 53191046 | 53191047 | 13 | 89179385 | 89199714 | 15 |
| 96520233 | 96520234 | 14 | 27324990 | 27376099 | 16 |
| 101932559 | 101932560 | 15 | 30194149 | 30200397 | 16 |
| 68645969 | 68645970 | 15 | 31271312 | 31344213 | 16 |
| 74466337 | 74466338 | 15 | 84599201 | 84651683 | 16 |
| 83776915 | 83776916 | 15 | 85932410 | 85956215 | 16 |
| 15923487 | 15923488 | 16 | 7239849 | 7254797 | 17 |
| 28079611 | 28079612 | 16 | 76126852 | 76139049 | 17 |
| 31129199 | 31129200 | 16 | 10381512 | 10397291 | 19 |
| 49312543 | 49312544 | 16 | 36395304 | 36399197 | 19 |
| 17832220 | 17832221 | 17 | 51874861 | 51875969 | 19 |
| 42151680 | 42151681 | 17 | 39314489 | 39317880 | 20 |
| 5019989 | 5019990 | 17 | 46305869 | 46351904 | 21 |
| 62294665 | 62294666 | 17 | 44568837 | 44615413 | 22 |

TABLE 3
Markers, given as nucleic acid sequence with start and end, that
show coordinated hyper/hypo methylation in HF/DCM in peripheral
blood and myocardial tissue (with regard to reference genome hg19)

| start | end | chr. | start | end | chr. |
|---|---|---|---|---|---|
| 2339980 | 2409222 | 11 | 171459078 | 171625390 | 5 |
| 2455915 | 2880339 | 11 | 176900396 | 176934607 | 5 |
| 81762703 | 82001899 | 16 | 33239788 | 33244287 | 6 |
| 84033273 | 84086241 | 16 | 75784043 | 75925767 | 6 |
| 82650409 | 83840204 | 16 | 157089064 | 157541913 | 6 |
| 31421065 | 31814916 | 18 | 99603205 | 99649312 | 7 |
| 32063255 | 32481808 | 18 | 139198603 | 139239730 | 7 |
| 30242635 | 30363025 | 18 | 157321751 | 158390480 | 7 |
| 6041527 | 6171253 | 1 | 37631710 | 37712414 | 8 |
| 40314411 | 40848193 | 2 | 41500740 | 41764280 | 8 |
| 239959865 | 240333348 | 2 | 42938659 | 42988577 | 8 |
| 140762242 | 141029076 | 9 | 54128285 | 54174257 | 8 |
| 12388733 | 12562348 | 11 | 14071843 | 14408982 | 9 |
| 98595913 | 98686551 | 13 | 17174254 | 17254053 | 10 |
| 24795228 | 24819251 | 14 | 42998959 | 43058270 | 10 |
| 24824880 | 24858810 | 14 | 95316423 | 95374237 | 10 |
| 3765056 | 3940727 | 16 | 108323422 | 108934292 | 10 |
| 70107162 | 70132561 | 17 | 7524530 | 7688358 | 11 |
| 45513264 | 45827492 | 20 | 75100531 | 75143324 | 11 |
| 50673613 | 50699834 | 22 | 117175274 | 117293984 | 11 |
| 32073288 | 32108119 | 1 | 49319507 | 49361334 | 12 |
| 32807123 | 32839913 | 1 | 74862597 | 74902805 | 14 |
| 41817595 | 41859262 | 1 | 85986489 | 86105034 | 14 |
| 53182127 | 53303014 | 1 | 45374849 | 45416542 | 15 |
| 66248198 | 66850259 | 1 | 69442924 | 69574556 | 15 |
| 111126203 | 111184096 | 1 | 84312839 | 84718594 | 15 |
| 151010217 | 151034462 | 1 | 47485035 | 47745434 | 16 |
| 176816439 | 177144109 | 1 | 55928605 | 56042684 | 17 |
| 28815 | 56870 | 2 | 77896143 | 78019647 | 17 |
| 5822800 | 5851516 | 2 | 78133792 | 78193130 | 17 |
| 43854413 | 44005126 | 2 | 5279019 | 5307052 | 18 |
| 11587545 | 11772220 | 3 | 10656481 | 11158587 | 18 |
| 42613333 | 42646606 | 3 | 52558741 | 52636739 | 18 |
| 62236541 | 62365005 | 3 | 36026498 | 36048428 | 19 |
| 190560667 | 190620218 | 3 | 49288320 | 49324286 | 19 |
| 166784411 | 167035047 | 4 | 57311446 | 57362096 | 19 |
| 137657625 | 137695416 | 5 | 40918370 | 41055064 | 21 |
| 170836661 | 170894627 | 5 | 38291665 | 38348829 | 21 |

TABLE 3a
Preferred markers, given as nucleic acid sequence with
start and end, that show coordinated hyper/hypo methylation
in HF/DCM in peripheral blood and myocardial tissue
(with regard to reference genome hg19)

| start | end | chr. | start | end | chr. |
|---|---|---|---|---|---|
| 2349980 | 2399222 | 11 | 171469078 | 171615390 | 5 |
| 2465915 | 2870339 | 11 | 176910396 | 176924607 | 5 |
| 81772703 | 81991899 | 16 | 33239788 | 33244287 | 6 |
| 84043273 | 84076241 | 16 | 75794043 | 75915767 | 6 |
| 82660409 | 83830204 | 16 | 157099064 | 157531913 | 6 |
| 31431065 | 31804916 | 18 | 99613205 | 99639312 | 7 |
| 32073255 | 32471808 | 18 | 139208603 | 139229730 | 7 |
| 30252635 | 30353025 | 18 | 157331751 | 158380480 | 7 |
| 6051527 | 6161253 | 1 | 37641710 | 37702414 | 8 |
| 40324411 | 40838193 | 2 | 41510740 | 41754280 | 8 |
| 239969865 | 240323348 | 2 | 42948659 | 42978577 | 8 |
| 140772242 | 141019076 | 9 | 54138285 | 54164257 | 8 |
| 12398733 | 12552348 | 11 | 14081843 | 14398982 | 9 |
| 98605913 | 98676551 | 13 | 17184254 | 17244053 | 10 |
| 24805228 | 24809251 | 14 | 43008959 | 43048270 | 10 |
| 24834880 | 24848810 | 14 | 95326423 | 95364237 | 10 |
| 3775056 | 3930727 | 16 | 108333422 | 108924292 | 10 |
| 70117162 | 70122561 | 17 | 7534530 | 7678358 | 11 |
| 45523264 | 45817492 | 20 | 75110531 | 75133324 | 11 |
| 50683613 | 50689834 | 22 | 117185274 | 117283984 | 11 |
| 32083288 | 32098119 | 1 | 49329507 | 49351334 | 12 |
| 32817123 | 32829913 | 1 | 74872597 | 74892805 | 14 |
| 41827595 | 41849262 | 1 | 85996489 | 86095034 | 14 |
| 53192127 | 53293014 | 1 | 45384849 | 45406542 | 15 |
| 66258198 | 66840259 | 1 | 69452924 | 69564556 | 15 |
| 111136203 | 111174096 | 1 | 84322839 | 84708594 | 15 |
| 151020217 | 151024462 | 1 | 47495035 | 47735434 | 16 |
| 176826439 | 177134109 | 1 | 55938605 | 56032684 | 17 |
| 38815 | 46870 | 2 | 77906143 | 78009647 | 17 |
| 5832800 | 5841516 | 2 | 78143792 | 78183130 | 17 |
| 43864413 | 43995126 | 2 | 5289019 | 5297052 | 18 |
| 11597545 | 11762220 | 3 | 10666481 | 11148587 | 18 |
| 42623333 | 42636606 | 3 | 52568741 | 52626739 | 18 |
| 62246541 | 62355005 | 3 | 36036498 | 36038428 | 19 |
| 190570667 | 190610218 | 3 | 49298320 | 49314286 | 19 |
| 166794411 | 167025047 | 4 | 57321446 | 57352096 | 19 |
| 137667625 | 137685416 | 5 | 40928370 | 41045064 | 21 |
| 170846661 | 170884627 | 5 | 38301665 | 38338829 | 21 |

TABLE 3b
Particularly preferred markers, given as nucleic acid
sequence with start and end, that show coordinated hyper/hypo
methylation in HF/DCM in peripheral blood and myocardial
tissue (with regard to reference genome hg19)

| start | end | chr. | start | end | chr. |
|---|---|---|---|---|---|
| 2368070 | 2368071 | 11 | 170848039 | 170848040 | 5 |
| 2376275 | 2376276 | 11 | 171469429 | 171469430 | 5 |
| 2594153 | 2594154 | 11 | 176924827 | 176924828 | 5 |
| 2594840 | 2594841 | 11 | 33241974 | 33241975 | 6 |
| 2690304 | 2690305 | 11 | 75798778 | 75798779 | 6 |
| 81806083 | 81806084 | 16 | 157342220 | 157342221 | 6 |
| 84076320 | 84076321 | 16 | 99627985 | 99627986 | 7 |
| 82970452 | 82970453 | 16 | 139208852 | 139208853 | 7 |
| 31805151 | 31805152 | 18 | 157452656 | 157452657 | 7 |
| 32173093 | 32173094 | 18 | 37655503 | 37655504 | 8 |
| 30351983 | 30351984 | 18 | 41625127 | 41625128 | 8 |
| 6146988 | 6146989 | 1 | 42948547 | 42948548 | 8 |
| 40678691 | 40678692 | 2 | 54164391 | 54164392 | 8 |
| 240082420 | 240082421 | 2 | 14313043 | 14313044 | 9 |
| 140773129 | 140773130 | 9 | 17183411 | 17183412 | 10 |
| 12524208 | 12524209 | 11 | 43048646 | 43048647 | 10 |
| 98605951 | 98605952 | 13 | 95326974 | 95326975 | 10 |
| 24804339 | 24804340 | 14 | 108924398 | 108924399 | 10 |
| 24836148 | 24836149 | 14 | 7535256 | 7535257 | 11 |
| 3824553 | 3824554 | 16 | 75110505 | 75110506 | 11 |
| 70117522 | 70117523 | 17 | 117283767 | 117283768 | 11 |
| 45523996 | 45523997 | 20 | 49330158 | 49330159 | 12 |
| 50689804 | 50689805 | 22 | 74892569 | 74892570 | 14 |
| 32083535 | 32083536 | 1 | 85999731 | 85999732 | 14 |
| 32827834 | 32827835 | 1 | 85999933 | 85999934 | 14 |
| 41827960 | 41827961 | 1 | 45404157 | 45404158 | 15 |
| 53238307 | 53238308 | 1 | 69452537 | 69452538 | 15 |
| 66259081 | 66259082 | 1 | 84323154 | 84323155 | 15 |
| 111148984 | 111148985 | 1 | 47494711 | 47494712 | 16 |
| 151019727 | 151019728 | 1 | 55952063 | 55952064 | 17 |
| 177034184 | 177034185 | 1 | 77951858 | 77951859 | 17 |
| 47150 | 47151 | 2 | 78152051 | 78152052 | 17 |
| 5836181 | 5836182 | 2 | 5295760 | 5295761 | 18 |
| 43986106 | 43986107 | 2 | 11148769 | 11148770 | 18 |
| 11623526 | 11623527 | 3 | 52625368 | 52625369 | 18 |
| 42626083 | 42626084 | 3 | 36036028 | 36036029 | 19 |
| 62354546 | 62354547 | 3 | 49306842 | 49306843 | 19 |
| 190580644 | 190580645 | 3 | 57352269 | 57352270 | 19 |
| 166797526 | 166797527 | 4 | 40984780 | 40984781 | 21 |
| 137674194 | 137674195 | 5 | 38337780 | 38337781 | 22 |

ID numbers for the methylation (methyl. ID) refer to the Infinium HumanMethylation450 BeadChip Kit probe IDs as listed in the HumanMethylation450 v1.2 Manifest (http://support.illumina.com/downloads/infinium_humanmethylation450_product_files.html), preferred reading directions for the respective double helix strand (str.; + or −) with regard to the reference genome for the genes as well as gene names, gene ensemble IDs (gene ID) and chromosomes (chr.) are found in Tables 1c, 2c and 2d, and 3c for Tables 1, 1a, 1b; 2, 2a, 2b; and 3, 3a, and 3b, respectively. Also, the starts and ends are given, with the respective tables in brackets. It should be noted that table 2, respectively 2a and 2b, has been split in two tables 2c and 2d, since for Table 2d the whole region has been shown to be significantly deregulated on methylation and expression level. Further, gene IDs, gene names and chromosomes are also given in Tables 4, 6, 8 and 10. In Tables 5, 7 and 9 cpg IDs—representing methylation locations (representing either a nucleobase or a paired nucleobase)—are given with regard to the Infinium HumanMethylation450K database, and chromosomes and positions (pos) are given with regard to the reference genome.

TABLE 4
Markers, given as nucleic acid sequence with start
and end, that show dysmethylation in HF/DCM in peripheral
blood (with regard to reference genome hg19)

| gene ID | gene name | chr. | start | end |
|---|---|---|---|---|
| ENSG00000176697 | BDNF | chr11 | 27686441 | 27753605 |
| ENSG00000137825 | ITPKA | chr15 | 41775592 | 41785747 |
| ENSG00000062524 | LTK | chr15 | 41805837 | 41816085 |
| ENSG00000165609 | NUDT5 | chr10 | 12217325 | 12248143 |
| ENSG00000151465 | CDC123 | chr10 | 12227965 | 12282588 |
| ENSG00000101493 | ZNF516 | chr18 | 74079645 | 74217146 |
| ENSG00000198925 | ATG9A | chr2 | 220084495 | 220104439 |
| ENSG00000163521 | GLB1L | chr2 | 220111329 | 220120200 |
| ENSG00000163516 | ANKZF1 | chr2 | 220084480 | 220091391 |
| ENSG00000090376 | IRAK3 | chr12 | 66572660 | 66638402 |
| ENSG00000144567 | FAM134A | chr2 | 220030948 | 220040201 |
| ENSG00000115649 | CNPPD1 | chr2 | 220046620 | 220052828 |
| ENSG00000213901 | SLC23A3 | chr2 | 219950052 | 220045549 |
| ENSG00000155093 | PTPRN2 | chr7 | 157341751 | 158390480 |
| ENSG00000108641 | B9D1 | chr17 | 19250868 | 19291495 |
| ENSG00000188803 | SHISA6 | chr17 | 11134581 | 1145738 |

The markers in Table 4 represent genomic regions with 10 kb up/downstream of genes that show statistically significant, particularly the statistically most significant, validated dysmethylation in peripheral blood, particularly in independent discovery (41 DCM and 31 CTRL) and verification cohorts (9 DCM and 28 CTRL). (DCM=dilated cardiomyopathy; CTRL=control)

TABLE 5
Markers, given as cpg ID with regard to the reference Infinium
HumanMethylation450K database, and as position (pos),
given with regard to the reference genome hg19, that show
dysmethylation in HF/DCM in peripheral blood

| cpg ID | chr. | pos |
|---|---|---|
| cg01642653 | chr11 | 27743476 |
| cg03177551 | chr15 | 41794747 |
| cg06109724 | chr10 | 12237553 |
| cg06688621 | chr18 | 74062785 |
| cg10545083 | chr2 | 220094517 |
| cg13807985 | chr12 | 66583255 |
| cg18822719 | chr2 | 220035962 |
| cg23618588 | chr7 | 158286570 |
| cg24884140 | chr17 | 19250190 |
| cg25215117 | chr17 | 11461665 |

The markers in Table 5 represent distinct cpg IDs and genomic positions (particularly top 10) that show statistically significant, particularly the statistically most significant, validated dysmethylation in peripheral blood, particularly in independent discovery (41 DCM and 31 CTRL) and verification cohorts (9 DCM and 28 CTRL).

TABLE 6
Markers, given as nucleic acid sequence with start
and end, that show dysmethylation in HF/DCM in peripheral
blood (with regard to reference genome hg19)

| Gene ID | gene name | chr. | start | end |
|---|---|---|---|---|
| ENSG00000167977 | KCTD5 | 16 | 2722477 | 2749031 |
| ENSG00000172382 | PRSS27 | 16 | 2772420 | 2780552 |
| ENSG00000221866 | PLXNA4 | 7 | 131818092 | 132343447 |
| ENSG00000108039 | XPNPEP1 | 10 | 111634525 | 111693311 |
| ENSG00000237976 | | 1 | 151309444 | 151310503 |
| ENSG00000143390 | RFX5 | 1 | 151323117 | 151329833 |
| ENSG00000064115 | TM7SF3 | 12 | 27136129 | 27177367 |
| ENSG00000144567 | FAM134A | 2 | 220030948 | 220040201 |
| ENSG00000115649 | CNPPD1 | 2 | 220046620 | 220052828 |
| ENSG00000213901 | SLC23A3 | 2 | 219950052 | 220045549 |
| ENSG00000100644 | HIF1A | 14 | 62152232 | 62204976 |
| ENSG00000258667 | HIF1A-AS2 | 14 | 62192277 | 62227815 |
| ENSG00000070540 | WIPI1 | 17 | 66427090 | 66463654 |
| ENSG00000141337 | ARSG | 17 | 66245324 | 66408872 |
| ENSG00000207561 | MIR635 | 17 | 66430593 | 66430689 |
| ENSG00000267009 | | 17 | 66399765 | 66511090 |
| ENSG00000145216 | FIP1L1 | 4 | 54233811 | 55151439 |

The markers in Table 6 represent genomic regions with 10 kb up/downstream of genes that show validated dysmethylation in peripheral blood, particularly in independent discovery (41 DCM and 31 CTRL) and verification cohorts (9 DCM and 28 CTRL) with an area under the curve (AUC) of more than 85% in the discovery and verification cohorts.

TABLE 7
Markers, given as cpg ID with regard to the reference Infinium
HumanMethylation450K database, and as position (pos),
given with regard to the reference genome hg19, that show
dysmethylation in HF/DCM in peripheral blood

| cpg ID | chr. | pos |
|---|---|---|
| cg04880804 | chr16 | 2762569 |
| cg06183123 | chr7 | 132340279 |
| cg11055926 | chr10 | 111683227 |
| cg11797228 | chr1 | 151319782 |
| cg12659065 | chr12 | 27156738 |
| cg18822719 | chr2 | 220035962 |
| cg20931965 | chr14 | 62186141 |
| cg27225708 | chr17 | 66420734 |
| cg27543103 | chr4 | 54975677 |

The markers in Table 7 represent distinct cpg IDs and genomic positions that show validated dysmethylation in peripheral blood, particularly in independent discovery (41 DCM and 31 CTRL) and verification cohorts (9 DCM and 28 CTRL) with an area under the curve (AUC) of more than 85% in the discovery and verification cohorts.

TABLE 8
Markers, given as nucleic acid sequence with start
and end, that show dysmethylation in HF/DCM in peripheral
blood (with regard to reference genome hg19)

| gene ID | gene name | chr. | start | end |
|---|---|---|---|---|
| ENSG00000134138 | MEIS2 | 15 | 37191407 | 37403504 |
| ENSG00000219438 | FAM19A5 | 22 | 48875273 | 49236724 |
| ENSG00000137309 | HMGA1 | 6 | 34194651 | 34204008 |
| ENSG00000156466 | GDF6 | 8 | 97164563 | 97183020 |
| ENSG00000124766 | SOX4 | 6 | 21583973 | 21588847 |
| ENSG00000007520 | TSR3 | 16 | 1409242 | 1411912 |
| ENSG00000090581 | GNPTG | 16 | 1391925 | 1403352 |
| ENSG00000007516 | BAIAP3 | 16 | 1373603 | 1389439 |
| ENSG00000132535 | DLG4 | 17 | 7103210 | 7133021 |
| ENSG00000072778 | ACADVL | 17 | 7110445 | 7118592 |
| ENSG00000199053 | MIR324 | 17 | 7136617 | 7136698 |
| ENSG00000004975 | DVL2 | 17 | 7138661 | 7147864 |
| ENSG00000236364 | | 1 | 165875117 | 165879920 |
| ENSG00000143179 | UCK2 | 1 | 165786769 | 165870855 |
| ENSG00000150907 | FOXO1 | 13 | 41139805 | 41250734 |
| ENSG00000115840 | SLC25A12 | 2 | 172650881 | 172874766 |
| ENSG00000128708 | HAT1 | 2 | 172768959 | 172838599 |
| ENSG00000002933 | TMEM176A | 7 | 150487492 | 150492208 |
| ENSG00000106565 | TMEM176B | 7 | 150498374 | 150508448 |
| ENSG00000009830 | POMT2 | 14 | 77751300 | 77797227 |
| ENSG00000100577 | GSTZ1 | 14 | 77777228 | 77787940 |
| ENSG00000122786 | CALD1 | 7 | 134419004 | 134645479 |
| ENSG00000091536 | MYO15A | 17 | 18002021 | 18073116 |
| ENSG00000129353 | SLC44A2 | 19 | 10703134 | 10745235 |
| ENSG00000129351 | ILF3 | 19 | 10754938 | 10793093 |
| ENSG00000267100 | ILF3-AS1 | 19 | 10772539 | 10774520 |
| ENSG00000163155 | LYSMD1 | 1 | 151142225 | 151148424 |
| ENSG00000163159 | VPS72 | 1 | 151152464 | 151177797 |
| ENSG00000163156 | SCNM1 | 1 | 151119141 | 151132773 |
| ENSG00000163154 | TNFAIP8L2 | 1 | 151119106 | 151122225 |
| ENSG00000234936 | | 2 | 43446713 | 43450533 |
| ENSG00000115970 | THADA | 2 | 43403801 | 43833185 |
| ENSG00000152518 | ZFP36L2 | 2 | 43459542 | 43463748 |
| ENSG00000198879 | SFMBT2 | 10 | 7210587 | 7463450 |
| ENSG00000178814 | OPLAH | 8 | 145116168 | 145128735 |
| ENSG00000128918 | ALDH1A2 | 15 | 58255623 | 58800065 |
| ENSG00000109180 | OCIAD1 | 4 | 48797230 | 48853834 |
| ENSG00000068383 | INPP5A | 10 | 134341325 | 134586979 |
| ENSG00000072657 | TRHDE | 12 | 72471047 | 73049422 |
| ENSG00000236333 | TRHDE-AS1 | 12 | 72657289 | 72678687 |
| ENSG00000167977 | KCTD5 | 16 | 2722477 | 2749031 |
| ENSG00000172382 | PRSS27 | 16 | 2772420 | 2780552 |
| ENSG00000137691 | C11orf70 | 11 | 101908175 | 101945291 |
| ENSG00000075618 | FSCN1 | 7 | 5622440 | 5636286 |
| ENSG00000011275 | RNF216 | 7 | 5669679 | 5831370 |
| ENSG00000165609 | NUDT5 | 10 | 12217325 | 12248143 |
| ENSG00000151465 | CDC123 | 10 | 12227965 | 12282588 |
| ENSG00000228989 | | 2 | 242619830 | 242623704 |
| ENSG00000168395 | ING5 | 2 | 242631451 | 242658893 |
| ENSG00000173083 | HPSE | 4 | 84223615 | 84266306 |
| ENSG00000173085 | COQ2 | 4 | 84192690 | 84216067 |
| ENSG00000221866 | PLXNA4 | 7 | 131818092 | 132343447 |
| ENSG00000240859 | | 7 | 139598 | 145465 |
| ENSG00000242474 | | 7 | 145854 | 159466 |
| ENSG00000165025 | SYK | 9 | 93554070 | 93650831 |
| ENSG00000125810 | CD93 | 20 | 23069987 | 23076977 |
| ENSG00000128917 | DLL4 | 15 | 41211539 | 41221237 |
| ENSG00000213719 | CLIC1 | 6 | 31708359 | 31717540 |
| ENSG00000211451 | GNRHR2 | 1 | 145519753 | 145526076 |
| ENSG00000131795 | RBM8A | 1 | 145497599 | 145503536 |
| ENSG00000197008 | ZNF138 | 7 | 64244767 | 64284054 |
| ENSG00000154122 | ANKH | 5 | 14714911 | 14881887 |
| ENSG00000266903 | | 19 | 45145501 | 45232031 |
| ENSG00000269834 | | 19 | 52902096 | 52911019 |
| ENSG00000167555 | ZNF528 | 19 | 52891103 | 52911665 |
| ENSG00000196730 | DAPK1 | 9 | 90102144 | 90313548 |
| ENSG00000090273 | NUDC | 1 | 27216730 | 27263353 |
| ENSG00000198746 | GPATCH3 | 1 | 27226980 | 27236957 |
| ENSG00000142751 | GPN2 | 1 | 27212625 | 27226788 |
| ENSG00000153162 | BMP6 | 6 | 7717031 | 7871655 |
| ENSG00000239264 | TXNDC5 | 6 | 7891484 | 8036646 |
| ENSG00000137203 | TFAP2A | 6 | 10403420 | 10429892 |
| ENSG00000106333 | PCOLCE | 7 | 100189801 | 100195798 |
| ENSG00000106336 | FBXO24 | 7 | 100171606 | 100188740 |
| ENSG00000224729 | PCOLCE- | 7 | 100197026 | 100211829 |
| | AS1 | | | |
| ENSG00000106330 | MOSPD3 | 7 | 100199726 | 100203007 |
| ENSG00000136271 | DDX56 | 7 | 44615017 | 44624650 |
| ENSG00000158604 | TMED4 | 7 | 44627494 | 44631886 |
| ENSG00000185215 | TNFAIP2 | 14 | 103579780 | 103593776 |
| ENSG00000163071 | SPATA18 | 4 | 52907498 | 52953458 |
| ENSG00000183060 | LYSMD4 | 15 | 100265903 | 100283766 |
| ENSG00000068305 | MEF2A | 15 | 100007371 | 100246671 |
| ENSG00000142453 | CARM1 | 19 | 10972190 | 11023453 |
| ENSG00000142444 | C19orf52 | 19 | 11029410 | 11034211 |
| ENSG00000130733 | YIPF2 | 19 | 11043445 | 11049357 |
| ENSG00000130159 | ECSIT | 19 | 11626732 | 11649989 |
| ENSG00000161914 | ZNF653 | 19 | 11604243 | 11626738 |
| ENSG00000135269 | TES | 7 | 115840548 | 115888837 |
| ENSG00000108039 | XPNPEP1 | 10 | 111634525 | 111693311 |
| ENSG00000155980 | KIF5A | 12 | 57933782 | 57970415 |
| ENSG00000175203 | DCTN2 | 12 | 57933886 | 57951114 |
| ENSG00000162415 | ZSWIM5 | 1 | 45492072 | 45781881 |
| ENSG00000233954 | | 1 | 16143680 | 16144194 |
| ENSG00000237976 | | 1 | 151309444 | 151310503 |
| ENSG00000143390 | RFX5 | 1 | 151323117 | 151329833 |
| ENSG00000204581 | | 2 | 111865923 | 111883165 |
| ENSG00000153094 | BCL2L11 | 2 | 111866956 | 111916024 |
| ENSG00000153093 | ACOXL | 2 | 111480151 | 111865799 |
| ENSG00000159692 | CTBP1 | 4 | 1215237 | 1253741 |
| ENSG00000064115 | TM7SF3 | 12 | 27136129 | 27177367 |
| ENSG00000113721 | PDGFRB | 5 | 149503401 | 149545435 |
| ENSG00000176095 | IP6K1 | 3 | 49771728 | 49833975 |
| ENSG00000204344 | STK19 | 6 | 31928869 | 31940598 |
| ENSG00000115339 | GALNT3 | 2 | 166614102 | 166661192 |
| ENSG00000170312 | CDK1 | 10 | 62528090 | 62544610 |
| ENSG00000005471 | ABCB4 | 7 | 87041014 | 87119751 |
| ENSG00000117143 | UAP1 | 1 | 162521324 | 162559627 |
| ENSG00000145506 | NKD2 | 5 | 998945 | 1029058 |
| ENSG00000169604 | ANTXR1 | 2 | 69230311 | 69466459 |
| ENSG00000140939 | NOL3 | 16 | 67194058 | 67199643 |
| ENSG00000179044 | EXOC3L1 | 16 | 67228270 | 67234107 |
| ENSG00000102878 | HSF4 | 16 | 67187289 | 67193848 |
| ENSG00000196123 | KIAA0895L | 16 | 67219506 | 67227943 |
| ENSG00000165138 | ANNS6 | 9 | 101503612 | 101569247 |
| ENSG00000133111 | RFXAP | 13 | 37383362 | 37393241 |
| ENSG00000160563 | MED27 | 9 | 134745495 | 134965295 |
| ENSG00000184465 | WDR27 | 6 | 169867308 | 170112159 |
| ENSG00000135094 | SDS | 12 | 113840251 | 113874106 |
| ENSG00000124831 | LRRFIP1 | 2 | 238526220 | 238712325 |
| ENSG00000106012 | IQCE | 7 | 2588633 | 2644368 |
| ENSG00000204463 | BAG6 | 6 | 31616806 | 31630482 |
| ENSG00000165355 | FBXO33 | 14 | 39876874 | 39911704 |
| ENSG00000197757 | HOXC6 | 12 | 54374409 | 54414607 |
| ENSG00000114316 | USP4 | 3 | 49325265 | 49388145 |
| ENSG00000237641 | | 2 | 232664192 | 232664597 |
| ENSG00000156973 | PDE6D | 2 | 232607136 | 232660982 |
| ENSG00000144524 | COPS7B | 2 | 232636382 | 232663963 |
| ENSG00000002587 | HS3ST1 | 4 | 11404775 | 11441389 |
| ENSG00000136238 | RAC1 | 7 | 6404155 | 6433608 |
| ENSG00000113387 | SUB1 | 5 | 32521740 | 32594185 |
| ENSG00000128652 | HOXD3 | 2 | 176991341 | 177027830 |
| ENSG00000144567 | FAM134A | 2 | 220030948 | 220040201 |
| ENSG00000115649 | CNPPD1 | 2 | 220046620 | 220052828 |
| ENSG00000213901 | SLC23A3 | 2 | 219950052 | 220045549 |
| ENSG00000152953 | STK32B | 4 | 5043170 | 5492725 |
| ENSG00000148814 | LRRC27 | 10 | 134135615 | 134185010 |
| ENSG00000011105 | TSPAN9 | 12 | 3176522 | 3385730 |
| ENSG00000139684 | ESD | 13 | 47355392 | 47381367 |
| ENSG00000182667 | NTM | 11 | 131230374 | 132196716 |
| ENSG00000133313 | CNDP2 | 18 | 72153052 | 72178366 |
| ENSG00000140506 | LMAN1L | 15 | 75095058 | 75108099 |
| ENSG00000261606 | | 15 | 75098565 | 75114136 |
| ENSG00000140474 | ULK3 | 15 | 75138458 | 75145687 |
| ENSG00000144744 | UBA3 | 3 | 69113882 | 69139559 |
| ENSG00000244513 | | 3 | 69053093 | 69095773 |
| ENSG00000144747 | TMF1 | 3 | 69078979 | 69111484 |
| ENSG00000073712 | FERMT2 | 14 | 53333987 | 53429153 |
| ENSG00000100644 | HIF1A | 14 | 62152232 | 62204976 |
| ENSG00000258667 | HIF1A-AS2 | 14 | 62192277 | 62227815 |
| ENSG00000106066 | CPVL | 7 | 29044848 | 29245067 |
| ENSG00000106069 | CHN2 | 7 | 29151891 | 29543944 |
| ENSG00000144649 | FAM198A | 3 | 43010760 | 43091703 |
| ENSG00000267282 | | 19 | 45395285 | 45404133 |
| ENSG00000130202 | PVRL2 | 19 | 45339433 | 45382485 |
| ENSG00000130204 | TOMM40 | 19 | 45383827 | 45396946 |
| ENSG00000126214 | KLC1 | 14 | 104018234 | 104157888 |
| ENSG00000162396 | PARS2 | 1 | 55232572 | 55240187 |
| ENSG00000139832 | RAB20 | 13 | 111185418 | 111224080 |
| ENSG00000182557 | SPNS3 | 17 | 4326984 | 4381503 |
| ENSG00000136720 | HS6ST1 | 2 | 129004291 | 129086151 |
| ENSG00000179348 | GATA2 | 3 | 128208271 | 128222028 |
| ENSG00000244300 | | 3 | 128198056 | 128211191 |
| ENSG00000065675 | PRKCQ | 10 | 6479106 | 6632263 |
| ENSG00000172428 | MYEOV2 | 2 | 241075981 | 241086224 |
| ENSG00000142459 | EVI5L | 19 | 7885120 | 7919862 |
| ENSG00000086827 | ZW10 | 11 | 113613910 | 113654533 |
| ENSG00000176973 | FAM89B | 11 | 65329821 | 65331669 |
| ENSG00000173465 | SSSCA1 | 11 | 65327902 | 65331413 |
| ENSG00000260233 | SSSCA1- | 11 | 65347132 | 65347744 |
| | AS1 | | | |
| ENSG00000173442 | EHBP1L1 | 11 | 65333510 | 65350121 |
| ENSG00000168056 | LTBP3 | 11 | 65316277 | 65336401 |
| ENSG00000233527 | | 19 | 37053973 | 37075610 |
| ENSG00000186020 | ZNF529 | 19 | 37035677 | 37106178 |
| ENSG00000152291 | TGOLN2 | 2 | 85555148 | 85565548 |
| ENSG00000198612 | COPS8 | 2 | 237983956 | 237999109 |
| ENSG00000227252 | | 2 | 237978078 | 238004460 |
| ENSG00000169398 | PTK2 | 8 | 141678000 | 142022315 |
| ENSG00000131473 | ACLY | 17 | 40033162 | 40096795 |
| ENSG00000145247 | OCIAD2 | 4 | 48897037 | 48918954 |
| ENSG00000111452 | GPR133 | 12 | 131428453 | 131616014 |
| ENSG00000099942 | CRKL | 22 | 21261715 | 21298037 |
| ENSG00000070540 | WIPI1 | 17 | 66427090 | 66463654 |
| ENSG00000141337 | ARSG | 17 | 66245324 | 66408872 |
| ENSG00000207561 | MIR635 | 17 | 66430593 | 66430689 |
| ENSG00000267009 | | 17 | 66399765 | 66511090 |
| ENSG00000154957 | ZNF18 | 17 | 11890757 | 11910827 |
| ENSG00000171217 | CLDN20 | 6 | 155575148 | 155587682 |
| ENSG00000235381 | | 6 | 155584274 | 155587858 |
| ENSG00000146426 | TIAM2 | 6 | 155143832 | 155568857 |
| ENSG00000029639 | TFB1M | 6 | 155588644 | 155645627 |
| ENSG00000145216 | FIP1L1 | 4 | 54233811 | 55151439 |

The markers in Table 8 represent genomic regions with 10 kb up/downstream of genes that show validated dysmethylation in peripheral blood, particularly in independent discovery (41 DCM and 31 CTRL) and verification cohorts (9 DCM and 28 CTRL) with an area under the curve (AUC) of more than 80% in the discovery and verification cohorts.

TABLE 9
Markers, given as cpg ID with regard to the reference Infinium
HumanMethylation450K database, and as position (pos),
given with regard to the reference genome hg19, that show
dysmethylation in HF/DCM in peripheral blood

| cpg ID | chr. | pos |
|---|---|---|
| cg00398764 | chr15 | 37402637 |
| cg00481629 | chr22 | 48972850 |
| cg00544436 | chr6 | 34203564 |
| cg00585714 | chr8 | 97156859 |
| cg00792966 | chr6 | 21595983 |
| cg01258653 | chr16 | 1393103 |
| cg01377644 | chr17 | 7126609 |
| cg01574241 | chr1 | 165873825 |
| cg01995660 | chr13 | 41238844 |
| cg02155405 | chr2 | 172776401 |
| cg02244695 | chr7 | 150497346 |
| cg02315508 | chr14 | 77787366 |
| cg02516134 | chr7 | 134575187 |
| cg02628561 | chr17 | 18061605 |
| cg03301945 | chr19 | 10764555 |
| cg03316474 | chr1 | 151138495 |
| cg03443205 | chr2 | 43454133 |
| cg03832371 | chr10 | 7290545 |
| cg03932271 | chr8 | 145111468 |
| cg04189295 | chr15 | 58653220 |
| cg04422289 | chr4 | 48833305 |
| cg04716580 | chr10 | 134546291 |
| cg04775889 | chr12 | 72665880 |
| cg04880804 | chr16 | 2762569 |
| cg05892674 | chr11 | 101918304 |
| cg06109226 | chr7 | 5650145 |
| cg06109724 | chr10 | 12237553 |
| cg06164187 | chr2 | 242641258 |
| cg06168319 | chr4 | 84205972 |
| cg06183123 | chr7 | 132340279 |
| cg06601579 | chr7 | 142966 |
| cg07160163 | chr9 | 93563778 |
| cg07286123 | chr20 | 23067126 |
| cg07431199 | chr15 | 41218265 |
| cg07584663 | chr6 | 31697834 |
| cg07600211 | chr1 | 145516081 |
| cg08135727 | chr7 | 64254733 |
| cg08482307 | chr5 | 14728684 |
| cg08485918 | chr19 | 45207541 |
| cg08525430 | chr19 | 52900882 |
| cg08797471 | chr9 | 90113120 |
| cg09174009 | chr1 | 27216796 |
| cg09245939 | chr6 | 7881428 |
| cg09288780 | chr6 | 10413394 |
| cg09326362 | chr7 | 100202679 |
| cg10045804 | chr7 | 44621958 |
| cg10367412 | chr14 | 103590195 |
| cg10418567 | chr4 | 52917567 |
| cg10620429 | chr15 | 100253266 |
| cg10706553 | chr19 | 11039446 |
| cg10707300 | chr19 | 11616032 |
| cg10728469 | chr7 | 115850755 |
| cg11055926 | chr10 | 111683227 |
| cg11087358 | chr12 | 57940980 |
| cg11155625 | chr1 | 45769710 |
| cg11650974 | chr1 | 16134399 |
| cg11797228 | chr1 | 151319782 |
| cg12427896 | chr2 | 111880694 |
| cg12525219 | chr4 | 1228640 |
| cg12659065 | chr12 | 27156738 |
| cg12727795 | chr5 | 149535695 |
| cg13033938 | chr3 | 49824475 |
| cg13116438 | chr6 | 31940606 |
| cg13169065 | chr2 | 166650947 |
| cg13227473 | chr10 | 62538143 |
| cg13338827 | chr7 | 87104932 |
| cg13471915 | chr1 | 162531167 |
| cg13621612 | chr5 | 1021202 |
| cg13766043 | chr2 | 69396932 |
| cg14174336 | chr16 | 67208654 |
| cg14281264 | chr9 | 101556171 |
| cg14522731 | chr13 | 37393990 |
| cg14573676 | chr9 | 134954987 |
| cg14582523 | chr6 | 169952299 |
| cg15277108 | chr12 | 113842998 |
| cg15579587 | chr2 | 238600061 |
| cg15776929 | chr7 | 2643444 |
| cg15875502 | chr6 | 31630077 |
| cg16507511 | chr14 | 39901950 |
| cg17026220 | chr12 | 54410580 |
| cg17336172 | chr3 | 49377548 |
| cg17355126 | chr2 | 232651397 |
| cg17997641 | chr4 | 11401872 |
| cg18404925 | chr7 | 6413861 |
| cg18721397 | chr5 | 32584912 |
| cg18750960 | chr2 | 177016417 |
| cg18822719 | chr2 | 220035962 |
| cg18827954 | chr4 | 5053585 |
| cg18878654 | chr10 | 134186874 |
| cg19182035 | chr12 | 3393005 |
| cg19196918 | chr13 | 47371267 |
| cg19417526 | chr11 | 131895599 |
| cg19523664 | chr18 | 72160077 |
| cg19785742 | chr15 | 75118821 |
| cg19821425 | chr3 | 69101663 |
| cg19909334 | chr14 | 53418212 |
| cg20931965 | chr14 | 62186141 |
| cg21110052 | chr7 | 29234262 |
| cg21396456 | chr3 | 43021214 |
| cg21549639 | chr19 | 45394156 |
| cg22353818 | chr14 | 104095074 |
| cg22693570 | chr1 | 55224579 |
| cg22983760 | chr13 | 111214246 |
| cg23288535 | chr17 | 4336494 |
| cg23366762 | chr2 | 128991292 |
| cg23520930 | chr3 | 128206967 |
| cg23875854 | chr10 | 6531368 |
| cg23973558 | chr2 | 241075520 |
| cg24411648 | chr19 | 7939467 |
| cg24427944 | chr11 | 113644552 |
| cg25010805 | chr11 | 65334385 |
| cg25445244 | chr19 | 37064171 |
| cg25654619 | chr2 | 85555411 |
| cg25656096 | chr2 | 237990400 |
| cg26099902 | chr8 | 141901449 |
| cg26476599 | chr17 | 40086761 |
| cg26731119 | chr4 | 48908849 |
| cg26829071 | chr12 | 131590596 |
| cg27088449 | chr22 | 21272634 |
| cg27225708 | chr17 | 66420734 |
| cg27296352 | chr17 | 11900707 |
| cg27383562 | chr6 | 155584850 |
| cg27543103 | chr4 | 54975677 |

The markers in Table 9 represent distinct cpg IDs and genomic positions that show validated dysmethylation in peripheral blood, particularly in independent discovery (41 DCM and 31 CTRL) and verification cohorts (9 DCM and 28 CTRL) with an area under the curve (AUC) of more than 80% in the discovery and verification cohorts.

TABLE 10
Markers, given as nucleic acid sequence with start and
end, that show dysmethylation in HF/DCM in peripheral
blood and myocardial tissue and are associated with RNA
expression levels (with regard to reference genome hg19)

| gene name | Chr. | start | end |
|---|---|---|---|
| NPPA | chr1 | 11915767 | 11918402 |
| NPPB | chr1 | 11927522 | 11928988 |

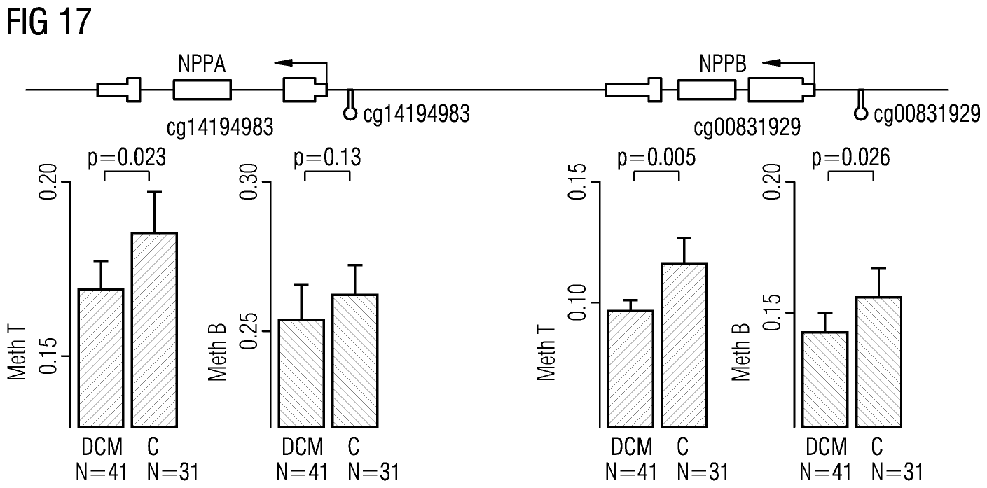

The markers in Table 10 represent markers that show dysmethylation in HF/DCM in peripheral blood and myocardial tissue and are associated with RNA expression levels and represent the genes NPPA and NPPB. The ANF and BNP loci encode atrial natriuretic factor (ANF) and brain natriuretic peptide (BNP), and the latter represents the present gold-standard biomarker for heart failure. The inventors found the same direction of dysmethylation in DNA, as also shown in FIG. 17 with regard to present Example 2, from heart tissue (red bars) and peripheral blood (blue bars). As expected, gene expression of NPPA (ANF) and NPPB is significantly dysregulated in the opposite direction in tissue (upregulation, p=0.0001 for both, data not shown) and transcript levels of NPPB highly correlate with NT-proBNP levels measured in plasma of the patients (R2=0.55). Accordingly, DNA methylation and RNA expression of both loci can serve as a biomarker for heart failure.

FIG. 17 shows therein the DNA methylation of the NPPA and NPPB locus. Natriuretic peptides are the gold-standard biomarkers in HF. In DCM, hypomethylation of the 5′ CpG is associated with increased expression. In blood, the same direction of dysmethylation is found representing a cross-tissue conservation. Hg19 coordinates for ANF (NPPA) and NPPB loci with 10 kb up/downstream window that can serve as biomarker for heart failure are given in table 10. Thus disclosed is also the usage of DNA methylation and RNA expression of ANF and BNP loci as biomarker for heart failure.

TABLE 1c
Summary of tables 1, 1a and 1b with additional data

| Methyl. ID | Start (1b) | End (1b) | Gene ID | Gene name | chr. | Start (1a) | End (1a) | str. | Start (1) | End (1) |
|---|---|---|---|---|---|---|---|---|---|---|
| cg03649649 | 56408197 | 56408198 | ENSG00000265206 | MIR142 | 17 | 56408246 | 56409869 | − | 56398246 | 56419869 |
| cg03649649 | 56408197 | 56408198 | ENSG00000265148 | BZRAP1-AS1 | 17 | 56402812 | 56493127 | + | 56392812 | 56503127 |
| cg06613515 | 77287656 | 77287657 | ENSG00000140368 | PSTPIP1 | 15 | 77285701 | 77329673 | + | 77275701 | 77339673 |
| cg10495227 | 82970452 | 82970453 | ENSG00000140945 | CDH13 | 16 | 82660409 | 83830204 | + | 82650409 | 83840204 |
| cg02856109 | 80531656 | 80531657 | ENSG00000066032 | CTNNA2 | 2 | 79412358 | 80875905 | + | 79402358 | 80885905 |
| cg02856109 | 80531656 | 80531657 | ENSG00000162951 | LRRTM1 | 2 | 80515484 | 80531874 | − | 80505484 | 80541874 |
| cg17033080 | 217508851 | 217508852 | ENSG00000115457 | IGFBP2 | 2 | 217497552 | 217529159 | + | 217487552 | 217539159 |
| cg20689294 | 129846082 | 129846083 | ENSG00000132334 | PTPRE | 10 | 129705326 | 129884119 | + | 129695326 | 129894119 |
| cg20720059 | 14772731 | 14772732 | ENSG00000162981 | FAM84A | 2 | 14772811 | 14790933 | + | 14762811 | 14800933 |
| cg16362232 | 430036 | 430037 | ENSG00000185101 | ANO9 | 11 | 417934 | 442011 | − | 407934 | 452011 |
| cg25943276 | 131533284 | 131533285 | ENSG00000182667 | NTM | 11 | 131240374 | 132206716 | + | 131230374 | 132216716 |
| cg24884140 | 19250190 | 19250191 | ENSG00000108641 | B9D1 | 17 | 19240868 | 19281495 | − | 19230868 | 19291495 |
| cg12115081 | 151038391 | 151038392 | ENSG00000170390 | DCLK2 | 4 | 150999427 | 151178609 | + | 150989427 | 151188609 |

TABLE 2c
Summary of tables 2, 2a and 2b (part 1) with additional data

| Methyl. ID | Start (2b) | End (2b) | Gene ID | Gene name | chr. | Start (2a) | End (2a) | str. | Start (2) | End (2) |
|---|---|---|---|---|---|---|---|---|---|---|
| cg24720355 | 119526255 | 119526256 | ENSG00000092607 | TBX15 | 1 | 119425670 | 119532179 | − | 119415670 | 119542179 |
| cg24144440 | 119526882 | 119526883 | ENSG00000092607 | TBX15 | 1 | 119425670 | 119532179 | − | 119415670 | 119542179 |
| cg02829688 | 119527008 | 119527009 | ENSG00000092607 | TBX15 | 1 | 119425670 | 119532179 | − | 119415670 | 119542179 |
| cg21647227 | 119527111 | 119527112 | ENSG00000092607 | TBX15 | 1 | 119425670 | 119532179 | − | 119415670 | 119542179 |
| cg05940231 | 119532189 | 119532190 | ENSG00000092607 | TBX15 | 1 | 119425670 | 119532179 | − | 119415670 | 119542179 |
| cg08942939 | 119532542 | 119532543 | ENSG00000092607 | TBX15 | 1 | 119425670 | 119532179 | − | 119415670 | 119542179 |
| cg21301805 | 119534644 | 119534645 | ENSG00000092607 | TBX15 | 1 | 119425670 | 119532179 | − | 119415670 | 119542179 |
| cg10587082 | 208293478 | 208293479 | ENSG00000076356 | PLXNA2 | 1 | 208195588 | 208417665 | − | 208185588 | 208427665 |
| cg01876531 | 208405868 | 208405869 | ENSG00000076356 | PLXNA2 | 1 | 208195588 | 208417665 | − | 208185588 | 208427665 |
| cg16045271 | 208412585 | 208412586 | ENSG00000076356 | PLXNA2 | 1 | 208195588 | 208417665 | − | 208185588 | 208427665 |
| cg04685570 | 114841202 | 114841203 | ENSG00000255399 | TBX5-AS1 | 12 | 114845997 | 114850636 | + | 114835997 | 114860636 |
| cg00182639 | 114841671 | 114841672 | ENSG00000255399 | TBX5-AS1 | 12 | 114845997 | 114850636 | + | 114835997 | 114860636 |
| cg00642359 | 114841708 | 114841709 | ENSG00000255399 | TBX5-AS1 | 12 | 114845997 | 114850636 | + | 114835997 | 114860636 |
| cg22045225 | 114841792 | 114841793 | ENSG00000255399 | TBX5-AS1 | 12 | 114845997 | 114850636 | + | 114835997 | 114860636 |
| cg21907579 | 114845868 | 114845869 | ENSG00000255399 | TBX5-AS1 | 12 | 114845997 | 114850636 | + | 114835997 | 114860636 |
| cg03877376 | 114846162 | 114846163 | ENSG00000255399 | TBX5-AS1 | 12 | 114845997 | 114850636 | + | 114835997 | 114860636 |
| cg03877376 | 114846162 | 114846163 | ENSG00000089225 | TBX5 | 12 | 114791737 | 114846247 | − | 114781737 | 114856247 |
| cg17645823 | 114846321 | 114846322 | ENSG00000255399 | TBX5-AS1 | 12 | 114845997 | 114850636 | + | 114835997 | 114860636 |
| cg17645823 | 114846321 | 114846322 | ENSG00000089225 | TBX5 | 12 | 114791737 | 114846247 | − | 114781737 | 114856247 |
| cg10281002 | 114846399 | 114846400 | ENSG00000255399 | TBX5-AS1 | 12 | 114845997 | 114850636 | + | 114835997 | 114860636 |
| cg10281002 | 114846399 | 114846400 | ENSG00000089225 | TBX5 | 12 | 114791737 | 114846247 | − | 114781737 | 114856247 |
| cg16458436 | 114846412 | 114846413 | ENSG00000255399 | TBX5-AS1 | 12 | 114845997 | 114850636 | + | 114835997 | 114860636 |
| cg16056219 | 75043777 | 75043778 | ENSG00000119681 | LTBP2 | 14 | 74964874 | 75079306 | − | 74954874 | 75089306 |
| cg14340889 | 75072120 | 75072121 | ENSG00000119681 | LTBP2 | 14 | 74964874 | 75079306 | − | 74954874 | 75089306 |
| cg08140459 | 75086513 | 75086514 | ENSG00000119681 | LTBP2 | 14 | 74964874 | 75079306 | − | 74954874 | 75089306 |
| cg09004195 | 222323493 | 222323494 | ENSG00000116106 | EPHA4 | 2 | 222282748 | 222438922 | − | 222272748 | 222448922 |
| cg13364311 | 222333289 | 222333290 | ENSG00000116106 | EPHA4 | 2 | 222282748 | 222438922 | − | 222272748 | 222448922 |
| cg03850035 | 222367110 | 222367111 | ENSG00000116106 | EPHA4 | 2 | 222282748 | 222438922 | − | 222272748 | 222448922 |
| cg01179095 | 11900652 | 11900653 | ENSG00000175206 | NPPA | 1 | 11905767 | 11908402 | − | 11895767 | 11918402 |
| cg03603260 | 151021364 | 151021365 | ENSG00000197622 | CDC42SE1 | 1 | 151023448 | 151042801 | − | 151013448 | 151052801 |
| cg13740187 | 154164699 | 154164700 | ENSG00000143549 | TPM3 | 1 | 154127785 | 154167124 | − | 154117785 | 154177124 |
| cg14529268 | 16335452 | 16335453 | ENSG00000183888 | C1orf64 | 1 | 16330732 | 16335302 | + | 16320732 | 16345302 |
| cg09013655 | 184005063 | 184005064 | ENSG00000198756 | COLGALT2 | 1 | 183898797 | 184006863 | − | 183888797 | 184016863 |
| cg01963906 | 27677240 | 27677241 | ENSG00000142765 | SYTL1 | 1 | 27668514 | 27680421 | + | 27658514 | 27690421 |
| cg16254946 | 54058616 | 54058617 | ENSG00000174332 | GLIS1 | 1 | 53971911 | 54199877 | − | 53961911 | 54209877 |
| cg08029603 | 854824 | 854825 | ENSG00000223764 | | 1 | 852246 | 856396 | − | 842246 | 866396 |
| cg09608533 | 125618188 | 125618189 | ENSG00000121898 | CPXM2 | 10 | 125465724 | 125699783 | − | 125455724 | 125709783 |
| cg04109883 | 50289110 | 50289111 | ENSG00000165633 | VSTM4 | 10 | 50222291 | 50323554 | − | 50212291 | 50333554 |
| cg00857536 | 50298306 | 50298307 | ENSG00000165633 | VSTM4 | 10 | 50222291 | 50323554 | − | 50212291 | 50333554 |
| cg24699895 | 71094286 | 71094287 | ENSG00000156515 | HK1 | 10 | 71029741 | 71161638 | + | 71019741 | 71171638 |
| cg02378006 | 73026288 | 73026289 | ENSG00000107731 | UNC5B | 10 | 72972328 | 73062621 | + | 72962328 | 73072621 |
| cg07216529 | 90712739 | 90712740 | ENSG00000138134 | STAMBPL1 | 10 | 90639492 | 90734910 | + | 90629492 | 90744910 |
| cg06595154 | 10716164 | 10716165 | ENSG00000072952 | MRVI1 | 11 | 10594639 | 10715535 | − | 10584639 | 10725535 |
| cg11822932 | 33913716 | 33913717 | ENSG00000135363 | LMO2 | 11 | 33880123 | 33913836 | − | 33870123 | 33923836 |
| cg02337873 | 65659393 | 65659394 | ENSG00000175602 | CCDC85B | 11 | 65657876 | 65659105 | + | 65647876 | 65669105 |
| cg21746120 | 68142234 | 68142235 | ENSG00000162337 | LRP5 | 11 | 68080078 | 68216743 | + | 68070078 | 68226743 |
| cg08679180 | 73034459 | 73034460 | ENSG00000110237 | ARHGEF17 | 11 | 73019335 | 73080136 | + | 73009335 | 73090136 |
| cg10630085 | 73108402 | 73108403 | ENSG00000054965 | FAM168A | 11 | 73111533 | 73309234 | − | 73101533 | 73319234 |
| cg15542639 | 93885254 | 93885255 | ENSG00000110218 | PANX1 | 11 | 93862095 | 93915138 | + | 93852095 | 93925138 |
| cg20735050 | 94521117 | 94521118 | ENSG00000166025 | AMOTL1 | 11 | 94439598 | 94609918 | + | 94429598 | 94619918 |
| cg24088496 | 96071506 | 96071507 | ENSG00000184384 | MAML2 | 11 | 95709763 | 96076344 | − | 95699763 | 96086344 |
| cg11513088 | 26111821 | 26111822 | ENSG00000123094 | RASSF8 | 12 | 26111963 | 26232825 | + | 26101963 | 26242825 |
| cg22070156 | 102104991 | 102104992 | ENSG00000198542 | ITGBL1 | 13 | 102104967 | 102375456 | + | 102094967 | 102385456 |
| cg07403350 | 108867111 | 108867112 | ENSG00000139826 | ABHD13 | 13 | 108870728 | 108886603 | + | 108860728 | 108896603 |
| cg02215357 | 53191046 | 53191047 | ENSG00000139675 | HNRNPA1L2 | 13 | 53191606 | 53217919 | + | 53181606 | 53227919 |
| cg19910802 | 96520233 | 96520234 | ENSG00000227051 | C14orf132 | 14 | 96505662 | 96560417 | + | 96495662 | 96570417 |
| cg27370471 | 101932559 | 101932560 | ENSG00000140479 | PCSK6 | 15 | 101840819 | 102065405 | − | 101830819 | 102075405 |
| cg05377733 | 68645969 | 68645970 | ENSG00000137809 | ITGA11 | 15 | 68594051 | 68724501 | − | 68584051 | 68734501 |
| cg17258195 | 74466337 | 74466338 | ENSG00000129009 | ISLR | 15 | 74466013 | 74469213 | + | 74456013 | 74479213 |
| cg27009545 | 83776915 | 83776916 | ENSG00000136404 | TM6SF1 | 15 | 83776160 | 83813606 | + | 83766160 | 83823606 |
| cg09284275 | 15923487 | 15923488 | ENSG00000133392 | MYH11 | 16 | 15797030 | 15950890 | − | 15787030 | 15960890 |
| cg04674421 | 28079611 | 28079612 | ENSG00000169181 | GSG1L | 16 | 27798851 | 28074830 | − | 27788851 | 28084830 |
| cg09509739 | 31129199 | 31129200 | ENSG00000262766 | | 16 | 31129400 | 31130068 | + | 31119400 | 31140068 |
| cg02696327 | 49312543 | 49312544 | ENSG00000102924 | CBLN1 | 16 | 49311829 | 49315742 | − | 49301829 | 49325742 |
| cg27232494 | 17832220 | 17832221 | ENSG00000175662 | TOM1L2 | 17 | 17746829 | 17875736 | − | 17736829 | 17885736 |
| cg26535547 | 42151680 | 42151681 | ENSG00000161654 | LSM12 | 17 | 42112004 | 42144987 | − | 42102004 | 42154987 |
| cg03995300 | 5019989 | 5019990 | ENSG00000129204 | USP6 | 17 | 5019734 | 5078329 | + | 5009734 | 5088329 |
| cg00864012 | 62294665 | 62294666 | ENSG00000136478 | TEX2 | 17 | 62224588 | 62340661 | − | 62214588 | 62350661 |
| cg06331359 | 78190755 | 78190756 | ENSG00000181045 | SLC26A11 | 17 | 78193499 | 78227299 | + | 78183499 | 78237299 |
| cg12475142 | 79012396 | 79012397 | ENSG00000226137 | BAIAP2-AS1 | 17 | 79002934 | 79008501 | − | 78992934 | 79018501 |
| cg22588546 | 8382941 | 8382942 | ENSG00000133026 | MYH10 | 17 | 8377524 | 8534079 | − | 8367524 | 8544079 |
| cg01085362 | 31848310 | 31848311 | ENSG00000121297 | TSHZ3 | 19 | 31765852 | 31840453 | − | 31755852 | 31850453 |
| cg09779027 | 7224513 | 7224514 | ENSG00000171105 | INSR | 19 | 7112267 | 7294045 | − | 7102267 | 7304045 |
| cg00428638 | 7224713 | 7224714 | ENSG00000171105 | INSR | 19 | 7112267 | 7294045 | − | 7102267 | 7304045 |
| cg07077013 | 177025198 | 177025199 | ENSG00000128652 | HOXD3 | 2 | 177001341 | 177037830 | + | 176991341 | 177047830 |
| cg10035294 | 223164925 | 223164926 | ENSG00000135903 | PAX3 | 2 | 223064608 | 223163715 | − | 223054608 | 223173715 |
| cg17245125 | 23843711 | 23843712 | ENSG00000119771 | KLHL29 | 2 | 23608089 | 23931481 | + | 23598089 | 23941481 |
| cg05403316 | 55339939 | 55339940 | ENSG00000115310 | RTN4 | 2 | 55199326 | 55339757 | − | 55189326 | 55349757 |
| cg16665041 | 3148787 | 3148788 | ENSG00000215251 | FASTKD5 | 20 | 3127166 | 3140543 | − | 3117166 | 3150543 |
| cg22164891 | 52199729 | 52199730 | ENSG00000171940 | ZNF217 | 20 | 52183605 | 52226446 | − | 52173605 | 52236446 |
| cg20979153 | 52199748 | 52199749 | ENSG00000171940 | ZNF217 | 20 | 52183605 | 52226446 | − | 52173605 | 52236446 |
| cg21172011 | 36577638 | 36577639 | ENSG00000159216 | RUNX1 | 21 | 36160099 | 37376965 | − | 36150099 | 37386965 |
| cg14703829 | 20780298 | 20780299 | ENSG00000099910 | KLHL22 | 22 | 20783529 | 20850170 | − | 20773529 | 20860170 |
| cg01640635 | 38864868 | 38864869 | ENSG00000100196 | KDELR3 | 22 | 38864068 | 38879452 | + | 38854068 | 38889452 |
| cg13066481 | 123372199 | 123372200 | ENSG00000065534 | MYLK | 3 | 123328897 | 123603178 | − | 123318897 | 123613178 |
| cg18274619 | 127494852 | 127494853 | ENSG00000074416 | MGLL | 3 | 127407910 | 127542051 | − | 127397910 | 127552051 |
| cg20950633 | 15540137 | 15540138 | ENSG00000206561 | COLQ | 3 | 15491641 | 15563258 | − | 15481641 | 15573258 |
| cg00434119 | 186080868 | 186080869 | ENSG00000058866 | DGKG | 3 | 185823458 | 186080026 | − | 185813458 | 186090026 |
| cg10960375 | 42694144 | 42694145 | ENSG00000114853 | ZBTB47 | 3 | 42695177 | 42709072 | + | 42685177 | 42719072 |
| cg02316506 | 42694803 | 42694804 | ENSG00000114853 | ZBTB47 | 3 | 42695177 | 42709072 | + | 42685177 | 42719072 |
| cg24074783 | 43405624 | 43405625 | ENSG00000163788 | SNRK | 3 | 43328005 | 43466256 | + | 43318005 | 43476256 |
| cg08052292 | 56789178 | 56789179 | ENSG00000163947 | ARHGEF3 | 3 | 56761447 | 57113357 | − | 56751447 | 57123357 |
| cg09427605 | 146740968 | 146740969 | ENSG00000151612 | ZNF827 | 4 | 146678780 | 146859787 | − | 146668780 | 146869787 |
| cg19116959 | 146841472 | 146841473 | ENSG00000151612 | ZNF827 | 4 | 146678780 | 146859787 | − | 146668780 | 146869787 |
| cg25924602 | 15397288 | 15397289 | ENSG00000163145 | C1QTNF7 | 4 | 15341443 | 15447790 | + | 15331443 | 15457790 |
| cg13832772 | 186283800 | 186283801 | ENSG00000109771 | LRP2BP | 4 | 186285033 | 186317053 | − | 186275033 | 186327053 |
| cg23664174 | 54357316 | 54357317 | ENSG00000072201 | LNX1 | 4 | 54325469 | 54567572 | − | 54315469 | 54577572 |
| cg14855841 | 76945459 | 76945460 | ENSG00000169248 | CXCL11 | 4 | 76954836 | 76962568 | − | 76944836 | 76972568 |
| cg21631086 | 138718914 | 138718915 | ENSG00000228672 | PROB1 | 5 | 138727636 | 138730885 | − | 138717636 | 138740885 |
| cg11462252 | 168139607 | 168139608 | ENSG00000184347 | SLIT3 | 5 | 168088746 | 168728133 | − | 168078746 | 168738133 |
| cg13112511 | 58882753 | 58882754 | ENSG00000113448 | PDE4D | 5 | 58264866 | 59817947 | − | 58254866 | 59827947 |
| cg02511723 | 71402031 | 71402032 | ENSG00000131711 | MAP1B | 5 | 71403062 | 71505395 | + | 71393062 | 71515395 |
| cg25515801 | 33240333 | 33240334 | ENSG00000231500 | RPS18 | 6 | 33239788 | 33244287 | + | 33229788 | 33254287 |
| cg04201373 | 33551533 | 33551534 | ENSG00000030110 | BAK1 | 6 | 33540330 | 33548019 | − | 33530330 | 33558019 |
| cg00604356 | 106507474 | 106507475 | ENSG00000105851 | PIK3CG | 7 | 106505724 | 106547590 | + | 106495724 | 106557590 |
| cg09374838 | 149578384 | 149578385 | ENSG00000204934 | ATP6V0E2- | 7 | 149564787 | 149577699 | − | 149554787 | 149587699 |
| | | | | AS1 | | | | | | |
| cg09374838 | 149578384 | 149578385 | ENSG00000171130 | ATP6V0E2 | 7 | 149570058 | 149577784 | + | 149560058 | 149587784 |
| cg26672672 | 47479433 | 47479434 | ENSG00000136205 | TNS3 | 7 | 47314753 | 47622156 | − | 47304753 | 47632156 |
| cg03143486 | 811491 | 811492 | ENSG00000164818 | HEATR2 | 7 | 766339 | 829190 | + | 756339 | 839190 |
| cg11201447 | 128808063 | 128808064 | ENSG00000249859 | PVT1 | 8 | 128806780 | 129113499 | + | 128796780 | 129123499 |
| cg25079691 | 25908057 | 25908058 | ENSG00000221818 | EBF2 | 8 | 25699247 | 25902913 | − | 25689247 | 25912913 |
| cg04244354 | 25908279 | 25908280 | ENSG00000221818 | EBF2 | 8 | 25699247 | 25902913 | − | 25689247 | 25912913 |
| cg12563372 | 25908503 | 25908504 | ENSG00000221818 | EBF2 | 8 | 25699247 | 25902913 | − | 25689247 | 25912913 |
| cg14523204 | 116359818 | 116359819 | ENSG00000138835 | RGS3 | 9 | 116207012 | 116360018 | + | 116197012 | 116370018 |

TABLE 2d
Summary of tables 2, 2a and 2b (part 2) with additional data

| Gene ID | Gene name | Chr. | Start (2a, 2b) | End (2a, 2b) | str. | Start (2) | End (2) |
|---|---|---|---|---|---|---|---|
| ENSG00000171608 | PIK3CD | 1 | 9711791 | 9789172 | + | 9701791 | 9799172 |
| ENSG00000130775 | THEMIS2 | 1 | 28199056 | 28213196 | + | 28189056 | 28223196 |
| ENSG00000081237 | PTPRC | 1 | 198607802 | 198726545 | + | 198597802 | 198736545 |
| ENSG00000115956 | PLEK | 2 | 68592306 | 68624585 | + | 68582306 | 68634585 |
| ENSG00000188042 | ARL4C | 2 | 235401686 | 235405697 | − | 235391686 | 235415697 |
| ENSG00000066336 | SPI1 | 11 | 47376412 | 47400127 | − | 47366412 | 47410127 |
| ENSG00000149781 | FERMT3 | 11 | 63974151 | 63991354 | + | 63964151 | 64001354 |
| ENSG00000136167 | LCP1 | 13 | 46700056 | 46786006 | − | 46690056 | 46796006 |
| ENSG00000172183 | ISG20 | 15 | 89179385 | 89199714 | + | 89169385 | 89209714 |
| ENSG00000077238 | IL4R | 16 | 27324990 | 27376099 | + | 27314990 | 27386099 |
| ENSG00000102879 | CORO1A | 16 | 30194149 | 30200397 | + | 30184149 | 30210397 |
| ENSG00000169896 | ITGAM | 16 | 31271312 | 31344213 | + | 31261312 | 31354213 |
| ENSG00000103187 | COTL1 | 16 | 84599201 | 84651683 | − | 84589201 | 84661683 |
| ENSG00000140968 | IRF8 | 16 | 85932410 | 85956215 | + | 85922410 | 85966215 |
| ENSG00000072818 | ACAP1 | 17 | 7239849 | 7254797 | + | 7229849 | 7264797 |
| ENSG00000167895 | TMC8 | 17 | 76126852 | 76139049 | + | 76116852 | 76149049 |
| ENSG00000090339 | ICAM1 | 19 | 10381512 | 10397291 | + | 10371512 | 10407291 |
| ENSG00000011600 | TYROBP | 19 | 36395304 | 36399197 | − | 36385304 | 36409197 |
| ENSG00000105374 | NKG7 | 19 | 51874861 | 51875969 | − | 51864861 | 51885969 |
| ENSG00000204103 | MAFB | 20 | 39314489 | 39317880 | − | 39304489 | 39327880 |
| ENSG00000160255 | ITGB2 | 21 | 46305869 | 46351904 | − | 46295869 | 46361904 |
| ENSG00000138964 | PARVG | 22 | 44568837 | 44615413 | + | 44558837 | 44625413 |

TABLE 3c
Summary of tables 3, 3a and 3b with additional data

| Methyl. ID | Start (3b) | End (3b) | Gene ID | Gene name | Chr. | Start (3a) | End (3a) | str. | Start (3) | End (3) |
|---|---|---|---|---|---|---|---|---|---|---|
| cg05532869 | 2368070 | 2368071 | ENSG00000238184 | CD81-AS1 | 11 | 2349980 | 2399222 | − | 2339980 | 2409222 |
| cg12121166 | 2376275 | 2376276 | ENSG00000238184 | CD81-AS1 | 11 | 2349980 | 2399222 | − | 2339980 | 2409222 |
| cg20751395 | 2594153 | 2594154 | ENSG00000053918 | KCNQ1 | 11 | 2465915 | 2870339 | + | 2455915 | 2880339 |
| cg13145504 | 2594840 | 2594841 | ENSG00000053918 | KCNQ1 | 11 | 2465915 | 2870339 | + | 2455915 | 2880339 |
| cg22239603 | 2690304 | 2690305 | ENSG00000053918 | KCNQ1 | 11 | 2465915 | 2870339 | + | 2455915 | 2880339 |
| cg21522797 | 81806083 | 81806084 | ENSG00000197943 | PLCG2 | 16 | 81772703 | 81991899 | + | 81762703 | 82001899 |
| cg02516845 | 84076320 | 84076321 | ENSG00000166558 | SIC38A8 | 16 | 84043273 | 84076241 | − | 84033273 | 84086241 |
| cg10495227 | 82970452 | 82970453 | ENSG00000140945 | CDH13 | 16 | 82660409 | 83830204 | + | 82650409 | 83840204 |
| cg25794153 | 31805151 | 31805152 | ENSG00000101746 | NOL4 | 18 | 31431065 | 31804916 | − | 31421065 | 31814916 |
| cg26530706 | 32173093 | 32173094 | ENSG00000134769 | DTNA | 18 | 32073255 | 32471808 | + | 32063255 | 32481808 |
| cg22648949 | 30351983 | 30351984 | ENSG00000197705 | KLHL14 | 18 | 30252635 | 30353025 | − | 30242635 | 30363025 |
| cg24068761 | 6146988 | 6146989 | ENSG00000069424 | KCNAB2 | 1 | 6051527 | 6161253 | + | 6041527 | 6171253 |
| cg12748607 | 40678691 | 40678692 | ENSG00000183023 | SIC8A1 | 2 | 40324411 | 40838193 | − | 40314411 | 40848193 |
| cg09283977 | 240082420 | 240082421 | ENSG00000068024 | HDAC4 | 2 | 239969865 | 240323348 | − | 239959865 | 240333348 |
| cg03912954 | 140773129 | 140773130 | ENSG00000148408 | CACNA1B | 9 | 140772242 | 141019076 | + | 140762242 | 141029076 |
| cg01744056 | 12524208 | 12524209 | ENSG00000197702 | PARVA | 11 | 12398733 | 12552348 | + | 12388733 | 12562348 |
| cg16932472 | 98605951 | 98605952 | ENSG00000065150 | IPO5 | 13 | 98605913 | 98676551 | + | 98595913 | 98686551 |
| cg14287235 | 24804339 | 24804340 | ENSG00000129465 | RIPK3 | 14 | 24805228 | 24809251 | − | 24795228 | 24819251 |
| cg25076767 | 24836148 | 24836149 | ENSG00000100968 | NFATC4 | 14 | 24834880 | 24848810 | + | 24824880 | 24858810 |
| cg03368634 | 3824553 | 3824554 | ENSG00000005339 | CREBBP | 16 | 3775056 | 3930727 | − | 3765056 | 3940727 |
| cg03547745 | 70117522 | 70117523 | ENSG00000125398 | SOX9 | 17 | 70117162 | 70122561 | + | 70107162 | 70132561 |
| cg09306675 | 45523996 | 45523997 | ENSG00000064655 | EYA2 | 20 | 45523264 | 45817492 | + | 45513264 | 45827492 |
| cg26943378 | 50689804 | 50689805 | ENSG00000100429 | HDAC10 | 22 | 50683613 | 50689834 | − | 50673613 | 50699834 |
| cg05536984 | 32083535 | 32083536 | ENSG00000121764 | HCRTR1 | 1 | 32083288 | 32098119 | + | 32073288 | 32108119 |
| cg15061530 | 32827834 | 32827835 | ENSG00000162526 | TSSK3 | 1 | 32817123 | 32829913 | + | 32807123 | 32839913 |
| cg12431729 | 41827960 | 41827961 | ENSG00000204060 | FOXO6 | 1 | 41827595 | 41849262 | + | 41817595 | 41859262 |
| cg11750103 | 53238307 | 53238308 | ENSG00000162378 | ZYG11B | 1 | 53192127 | 53293014 | + | 53182127 | 53303014 |
| cg26963271 | 66259081 | 66259082 | ENSG00000184588 | PDE4B | 1 | 66258198 | 66840259 | + | 66248198 | 66850259 |
| cg00791468 | 111148984 | 111148985 | ENSG00000177301 | KCNA2 | 1 | 111136203 | 111174096 | − | 111126203 | 111184096 |
| cg13072446 | 151019727 | 151019728 | ENSG00000143443 | C1orf56 | 1 | 151020217 | 151024462 | + | 151010217 | 151034462 |
| cg13474719 | 177034184 | 177034185 | ENSG00000152092 | ASTN1 | 1 | 176826439 | 177134109 | − | 176816439 | 177144109 |
| cg23548885 | 47150 | 47151 | ENSG00000184731 | FAM110C | 2 | 38815 | 46870 | − | 28815 | 56870 |
| cg26659079 | 5836181 | 5836182 | ENSG00000176887 | SOX11 | 2 | 5832800 | 5841516 | + | 5822800 | 5851516 |
| cg05939149 | 43986106 | 43986107 | ENSG00000152527 | PLEKHH2 | 2 | 43864413 | 43995126 | + | 43854413 | 44005126 |
| cg18809126 | 11623526 | 11623527 | ENSG00000144560 | VGLL4 | 3 | 11597545 | 11762220 | − | 11587545 | 11772220 |
| cg24823485 | 42626083 | 42626084 | ENSG00000008324 | SS18L2 | 3 | 42623333 | 42636606 | + | 42613333 | 42646606 |
| cg06327727 | 62354546 | 62354547 | ENSG00000241472 | PTPRG-AS1 | 3 | 62246541 | 62355005 | − | 62236541 | 62365005 |
| cg27338287 | 190580644 | 190580645 | ENSG00000205835 | GMNC | 3 | 190570667 | 190610218 | − | 190560667 | 190620218 |
| cg08923494 | 166797526 | 166797527 | ENSG00000038295 | TLL1 | 4 | 166794411 | 167025047 | + | 166784411 | 167035047 |
| cg14553364 | 137674194 | 137674195 | ENSG00000120709 | FAM53C | 5 | 137667625 | 137685416 | + | 137657625 | 137695416 |
| cg12364324 | 170848039 | 170848040 | ENSG00000156427 | FGF18 | 5 | 170846661 | 170884627 | + | 170836661 | 170894627 |
| cg26651429 | 171469429 | 171469430 | ENSG00000072786 | STK10 | 5 | 171469078 | 171615390 | − | 171459078 | 171625390 |
| cg13898548 | 176924827 | 176924828 | ENSG00000196923 | PDLIM7 | 5 | 176910396 | 176924607 | − | 176900396 | 176934607 |
| cg05560494 | 33241974 | 33241975 | ENSG00000096150 | RPS18 | 6 | 33239788 | 33244287 | + | 33229788 | 33254287 |
| cg15089846 | 75798778 | 75798779 | ENSG00000111799 | COL12A1 | 6 | 75794043 | 75915767 | − | 75784043 | 75925767 |
| cg26732340 | 157342220 | 157342221 | ENSG00000049618 | ARID1B | 6 | 157099064 | 157531913 | + | 157089064 | 157541913 |
| cg00155447 | 99627985 | 99627986 | ENSG00000106261 | ZKSCAN1 | 7 | 99613205 | 99639312 | + | 99603205 | 99649312 |
| cg08832906 | 139208852 | 139208853 | ENSG00000236279 | CLEC2L | 7 | 139208603 | 139229730 | + | 139198603 | 139239730 |
| cg00461149 | 157452656 | 157452657 | ENSG00000155093 | PTPRN2 | 7 | 157331751 | 158380480 | − | 157321751 | 158390480 |
| cg09121695 | 37655503 | 37655504 | ENSG00000020181 | GPR124 | 8 | 37641710 | 37702414 | + | 37631710 | 37712414 |
| cg09125812 | 41625127 | 41625128 | ENSG00000029534 | ANK1 | 8 | 41510740 | 41754280 | − | 41500740 | 41764280 |
| cg16587988 | 42948547 | 42948548 | ENSG00000185900 | POMK | 8 | 42948659 | 42978577 | + | 42938659 | 42988577 |
| cg16491617 | 54164391 | 54164392 | ENSG00000082556 | OPRK1 | 8 | 54138285 | 54164257 | − | 54128285 | 54174257 |
| cg01924448 | 14313043 | 14313044 | ENSG00000147862 | NFIB | 9 | 14081843 | 14398982 | − | 14071843 | 14408982 |
| cg24701032 | 17183411 | 17183412 | ENSG00000107614 | TRDMT1 | 10 | 17184254 | 17244053 | − | 17174254 | 17254053 |
| cg17003301 | 43048646 | 43048647 | ENSG00000234420 | ZNF37BP | 10 | 43008959 | 43048270 | − | 42998959 | 43058270 |
| cg25497250 | 95326974 | 95326975 | ENSG00000186188 | FFAR4 | 10 | 95326423 | 95364237 | + | 95316423 | 95374237 |
| cg26554592 | 108924398 | 108924399 | ENSG00000108018 | SORCS1 | 10 | 108333422 | 108924292 | − | 108323422 | 108934292 |
| cg12486121 | 7535256 | 7535257 | ENSG00000166387 | PPFIBP2 | 11 | 7534530 | 7678358 | + | 7524530 | 7688358 |
| cg19279432 | 75110505 | 75110506 | ENSG00000149273 | RPS3 | 11 | 75110531 | 75133324 | + | 75100531 | 75143324 |
| cg14727452 | 117283767 | 117283768 | ENSG00000110274 | CEP164 | 11 | 117185274 | 117283984 | + | 117175274 | 117293984 |
| cg07732097 | 49330158 | 49330159 | ENSG00000134287 | ARF3 | 12 | 49329507 | 49351334 | − | 49319507 | 49361334 |
| cg13222752 | 74892569 | 74892570 | ENSG00000183379 | SYNDIG1L | 14 | 74872597 | 74892805 | − | 74862597 | 74902805 |
| cg05295297 | 85999731 | 85999732 | ENSG00000185070 | FLRT2 | 14 | 85996489 | 86095034 | + | 85986489 | 86105034 |
| cg14400498 | 85999933 | 85999934 | ENSG00000185070 | FLRT2 | 14 | 85996489 | 86095034 | + | 85986489 | 86105034 |
| cg21883598 | 45404157 | 45404158 | ENSG00000140279 | DUOX2 | 15 | 45384849 | 45406542 | − | 45374849 | 45416542 |
| cg22381808 | 69452537 | 69452538 | ENSG00000138604 | GLCE | 15 | 69452924 | 69564556 | + | 69442924 | 69574556 |
| cg11611600 | 84323154 | 84323155 | ENSG00000156218 | ADAMTSL3 | 15 | 84322839 | 84708594 | + | 84312839 | 84718594 |
| cg01852244 | 47494711 | 47494712 | ENSG00000102893 | PHKB | 16 | 47495035 | 47735434 | + | 47485035 | 47745434 |
| cg07665510 | 55952063 | 55952064 | ENSG00000180891 | CUEDC1 | 17 | 55938605 | 56032684 | − | 55928605 | 56042684 |
| cg14787267 | 77951858 | 77951859 | ENSG00000167291 | TBC1D16 | 17 | 77906143 | 78009647 | − | 77896143 | 78019647 |
| cg14893129 | 78152051 | 78152052 | ENSG00000141527 | CARD14 | 17 | 78143792 | 78183130 | + | 78133792 | 78193130 |
| cg24498538 | 5295760 | 5295761 | ENSG00000198081 | ZBTB14 | 18 | 5289019 | 5297052 | − | 5279019 | 5307052 |
| cg24362812 | 11148769 | 11148770 | ENSG00000154864 | PIEZO2 | 18 | 10666481 | 11148587 | − | 10656481 | 11158587 |
| cg12113740 | 52625368 | 52625369 | ENSG00000166510 | CCDC68 | 18 | 52568741 | 52626739 | − | 52558741 | 52636739 |
| cg03124313 | 36036028 | 36036029 | ENSG00000105677 | TMEM147 | 19 | 36036498 | 36038428 | + | 36026498 | 36048428 |
| cg09430060 | 49306842 | 49306843 | ENSG00000105552 | BCAT2 | 19 | 49298320 | 49314286 | − | 49288320 | 49324286 |
| cg19098268 | 57352269 | 57352270 | ENSG00000198300 | PEG3 | 19 | 57321446 | 57352096 | − | 57311446 | 57362096 |
| cg08448665 | 40984780 | 40984781 | ENSG00000183778 | B3GALT5 | 21 | 40928370 | 41045064 | + | 40918370 | 41055064 |
| cg01777170 | 38337780 | 38337781 | ENSG00000100139 | MICALL1 | 22 | 38301665 | 38338829 | + | 38291665 | 38348829 |

According to certain embodiments, the presence of a plurality of markers is determined, so that the risk of heart failure and/or dilated cardiomyopathy can be determined more accurately.

A further aspect of the present invention is directed to the use of the markers in Table 1, Table 2, Table 3, Table 4, Table 5, Table 6, Table 7, Table 8, Table 9 and/or Table 10, preferably Table 1a, Table 2a, Table 3a, Table 4, Table 5, Table 6, Table 7, Table 8, Table 9 and/or Table 10, particularly preferably Table 1b, Table 2b, Table 3b, Table 4, Table 5, Table 6, Table 7, Table 8, Table 9 and/or Table 10, e.g. Table 1a, Table 2a and/or Table 3a, e.g. Table 1b, Table 2b and/or Table 3b, as a marker for heart failure and/or dilated cardiomyopathy in a patient.

Furthermore disclosed is a data bank comprising the markers disclosed in Table 1, Table 2, Table 3, Table 4, Table 5, Table 6, Table 7, Table 8, Table 9 and/or Table 10, preferably Table 1a, Table 2a, Table 3a, Table 4, Table 5, Table 6, Table 7, Table 8, Table 9 and/or Table 10, particularly preferably Table 1b, Table 2b and/or Table 3b, Table 4, Table 5, Table 6, Table 7, Table 8, Table 9 and/or Table 10, e.g. Table 1a, Table 2a and/or Table 3a, e.g. Table 1b, Table 2b and/or Table 3b.

According to certain embodiments, the data bank can be at a remote location and can be queried from a local client.

The present data banks can be used in a variety of applications. For example, the data bank can then be used, according to an aspect of the invention, in a method of determining a risk for heart failure and/or dilated cardiomyopathy in a patient.

Also disclosed is a data bank comprising markers obtained by the first and/or second aspect of the invention.

In addition, the present invention relates in a further aspect to a method of determining a risk for a disease in a patient, comprising

obtaining or providing data of an epigenomics profile and/or a transcriptome of at least one sample of the peripheral blood and/or a diseased tissue of the patient, and

determining the presence of at least one marker as determined by the method of the first and/or second aspect.

According to certain embodiments, the disease is heart failure (HF) and/or dilated cardiomyopathy (DCM), and the at least one marker as determined by the method of first and/or second aspect is at least a marker disclosed in Table 1, Table 2, Table 3, Table 4, Table 5, Table 6, Table 7, Table 8, Table 9 and/or Table 10, preferably Table 1a, Table 2a, Table 3a, Table 4, Table 5, Table 6, Table 7, Table 8, Table 9 and/or Table 10, particularly preferably Table 1b, Table 2b, Table 3b, Table 4, Table 5, Table 6, Table 7, Table 8, Table 9 and/or Table 10, e.g. Table 1a, Table 2a and/or Table 3a, e.g. Table 1b, Table 2b and/or Table 3b.

In a still further aspect the present invention relates to a computer program product comprising computer executable instructions which, when executed, perform a method of determining a risk for a disease in a patient.

In certain embodiments the computer program product is one on which program commands or program codes of a computer program for executing said method are stored. According to certain embodiments the computer program product is a storage medium.

The present invention also relates to the use of the computer program product in a method of determining a risk for a disease in a patient.

Further disclosed is a method of prognosis and/or for monitoring and/or assisting in drug-based therapy of patients diagnosed with heart failure and/or dilated cardiomyopathy, wherein a marker as disclosed in Table 1, Table 2, Table 3, Table 4, Table 5, Table 6, Table 7, Table 8, Table 9 and/or Table 10, preferably Table 1a, Table 2a, Table 3a, Table 4, Table 5, Table 6, Table 7, Table 8, Table 9 and/or Table 10, particularly preferably Table 1b, Table 2b, Table 3b, Table 4, Table 5, Table 6, Table 7, Table 8, Table 9 and/or Table 10, e.g. Table 1a, Table 2a and/or Table 3a, e.g. Table 1b, Table 2b and/or Table 3b, is used. The markers disclosed in Table 1, Table 2, Table 3, Table 4, Table 5, Table 6, Table 7, Table 8, Table 9 and/or Table 10, preferably Table 1a, Table 2a, Table 3a, Table 4, Table 5, Table 6, Table 7, Table 8, Table 9 and/or Table 10, particularly preferably Table 1b, Table 2b, Table 3b, Table 4, Table 5, Table 6, Table 7, Table 8, Table 9 and/or Table 10, e.g. Table 1a, Table 2a and/or Table 3a, e.g. Table 1b, Table 2b and/or Table 3b, allow a prognosis of the course of the disease as well as a monitoring thereof and can assist in deriving a conclusion regarding the medication prescription, etc., during the therapeutic treatment thereof.

### EXAMPLES

The present invention will now be described in detail with reference to several examples thereof. However, these examples are illustrative and do not limit the scope of the invention.

Some clinical perspectives are briefly discussed with regard to the Examples.

Clinical Perspective

1) What is new?

The application shows that Multi-omics studies allow detection of functional patterns in cardiovascular disease.

Epigenetic patterns are associated with heart failure due to dilated cardiomyopathy. The multi-omics studies design furthermore allowed detection of connected functional layers in cardiovascular disease.

DNA methylation of distinct genomic regions is conserved between heart tissue and peripheral blood. DNA methylation could represent a new class of heart failure biomarkers.

Transcriptional Regulation of natriuretic factors ANP and BNP is associated with conserved DNA methylation.

2) What are the clinical implications?

Epigenetic mechanisms are involved in chronic heart failure, which opens new perspectives for translational research. Their investigation as diagnostic, predictive of prognostic biomarkers and future drug targets needs further attention.

### Example 1

### Material and Methods

### Patient Enrollment and Study Design

The present study has been approved by the ethics committee, medical faculty of Heidelberg University. All participants have given written informed consent. The diagnosis of non-ischemic Dilated Cardiomyopathy (DCM) was confirmed by excluding relevant coronary artery disease (CAD) as determined by coronary angiography. Valvular heart disease was excluded by cMRI and/or echocardiography and myocarditis/inflammatory DCM by histopathology. Patients with history of uncontrolled hypertension, myocarditis, regular alcohol consumption or cardio-toxic chemotherapy were also excluded. To include the whole continuum of systolic heart failure, also early disease stages (EF<55%) who were symptomatic (dyspnoe, edema/congestion) were included.

After screening of n=135 DCM patients, n=38 met all inclusion and exclusion criteria and had sufficient amounts of high quality left ventricular biopsies (LV free wall) and peripheral blood samples available for high-throughput analyses. Control LV-biopsy specimens were obtained from patients after heart transplantation (n=31) that was at least 6 months ago, who had normal systolic and diastolic function and no evidence for relevant vasculopathy or acute/chronic organ rejection as judged by coronary angiography and immuno-histopathology. Additional gender- and age-matched controls for whole blood samples (n=31) had normal systolic and diastolic left ventricular function without evidence for other cardiovascular disease.

Additionally for further validation purposes, left ventricular myocardium of n=11 DCM patients who underwent heart transplantation and left ventricular myocardium (n=5) from previously healthy road accident victims were included.

In the mean, patients were 54 years old and disease onset was 11 months prior to inclusion. Detailed basic and clinical characteristics of DCM patients are summarized in the following Table 11.

TABLE 11
Detailed information of patients in the examples

| Patients' clinical characteristics | All patients |
|---|---|
| at the time of LV-EMB | (n = 38) |
| Basic characteristics | |
| Age, mean ± SD, y | 53.7 ± 12.6 |
| Age at onset ± SD, y | 52.8 ± 12.8 |
| Males, n. (%) | 30 (78.9%) |
| BMI, mean ± SD, kg/m² | 27 ± 5.6 |
| Diabetes, n. (%) | 3 (7.9%) |
| Atrial fibrillation, n. (%) | 5 (13%) |
| Dyspnoea, n. (%) | |
| NYHA I | 6 (16%) |
| NYHA II | 17 (46%) |
| NYHA III | 13 (35%) |
| NYHA IV | 1 (3%) |
| Family history of SCD or DCM, n. (%) | 8 (21%) |
| Laboratory tests | |
| White blood cell count, mean ± SD,/nl | 7.8 ± 2.4 |
| Haemoglobin, mean ± SD, g/dl | 14.4 ± 1.5 |
| eGFR, mean ± SD, mL/min/1.73 m² | 88.6 ± 16.3 |
| NT-proBNP, median (1Q; 3Q), ng/l | 767 (104; 2385) |
| hs-TNT, median (1Q; 3Q), pg/ml | 16 (8; 38) |
| Medications, n. (%) | |
| β-Blocker | 36 (95%) |
| ACE inhibitor or ARB | 38 (100%) |
| Loop diuretic | 17 (45%) |
| Aldosterone antagonist | 18 (47%) |
| Echocardiography | |
| LV ejection fraction, mean ± SD, % | 32 ± 15 |
| LV-EDD, mean ± SD, mm/m² MRI | 57 ± 9 |
| LV ejection fraction, mean ± SD, % | 37 ± 15 |
| LV-EDV index, mean ± SD, mL/m² | 130 ± 54 |
| LV-EDD index, mean ± SD, mm/m² | 31 ± 5 |
| RV-EDD index, mean ± SD, mm/m² | 24 ± 4 |

ACE, angiotensin-converting enzyme; ARB, angiotensin II receptor blocker; DCM, dilated cardiomyopathy; EDD: end-diastolic diameter; EDV: end-diastolic volume; GFR: Glomerular filtration rate; LV: left ventricular; LV-EMB: Left-Ventricular Endomyocardial Biopsy n: number; NYHA, New York Heart Association; SCD: sudden cardiac death; SD: standard deviation; 1Q: first quartile; 3Q: third Quartile.

### Biomaterial Processing

Biopsy specimens were obtained from the apical part of the free left ventricular wall (LV) from DCM patients or cardiac transplant patients (controls) undergoing cardiac catheterization using a standardized protocol. Biopsies were immediately washed in ice-cold saline (0.9% NaCl) and immediately transferred and stored in liquid nitrogen until DNA or RNA was extracted. After diagnostic workup of the biopsies (histopathology), remaining material was evenly dissected to isolate DNA and RNA. DNA was isolated from biopsies and peripheral blood using Qiagen DNA Blood Maxi Kit. Total RNA was extracted using the RNeasy kit according to the manufacturer's protocol (Qiagen, Germany) from biopsies and peripheral blood. RNA purity and concentration were determined using the Bioanalyzer 2100 (Agilent Technologies, Berkshire, UK) with a Eukaryote Total RNA Pico assay chip.

### DNA Methylation Profiling and RNA Sequencing

Methylation profiles were measured using the Illumina 450 k methylation assay, following procedures as described in Bibikova, M., et al.: High density DNA methylation array with single CpG site resolution, Genomics, 2011, 98(4): p. 288-95. From each patient, we subjected 200 ng DNA (blood) and 200 ng DNA (biopsy) to the measurements.

### Quality Control (QC) and Removal of Unreliable Measurements

Methylation sites with a detection p-value of >0.05 in more than 10% of the samples were removed from analysis. Methylation levels with a detection p-value of >0.05 in less than 10% of the samples were imputed via knn-imputation, as described in Hastie T, T., R, Narasimhan, B Chu, G, impute: impute: Imputation for microarray data, R package version 1.46.0, 2016. To reduce the effects of genomic variation on methylation measurements we excluded all methylation sites where we found variants in more than 10% of the DCM patients of the discovery cohort within the 50 bp probe region by whole genome sequencing. Methylation levels with variants in less than 10% of the DCM patients were imputed. We further removed all probes on X and Y chromosomes as well as probes which have been identified by Chen et al. (Chen, Y. A., et al., Discovery of cross-reactive probes and polymorphic CpGs in the Illumina Infinium HumanMethylation450 microarray. Epigenetics, 2013. 8(2): p. 203-9) to cross-hybridize with non-targeted DNA, yielding 394,247 methylation sites that passed QC. It should be noted that the predictive performance may even be increased when e.g. switching from the employed high-throughput Infinium HumanMethylation450 BeadChip screening array to a targeted analysis approach for single methylation sites.

### Whole Genome Sequencing

1 μg of total gDNA (genomic DNA) was sheared using the Covaris™ 5220 system, applying 2 treatments of 60 seconds each (peak power=140; duty factor=10) with 200 cycles/burst. 500 ng of sheared gDNA was taken and whole genome libraries were prepared using TruSeq DNA sample preparation kit according to manufacturer's protocols (Illumina, San Diego, US). Sequencing was performed on an IlluminaHiSeq 2000, using TruSeq SBS Kit v3 and reading two times 100 bp for paired end sequencing, on four lanes of a sequencing flowcell.

Demultiplexing of the raw sequencing reads and generation of the fastq files was done using CASAVA v.1.82. The raw reads were then mapped to the human reference genome (GRCh37/hg19, http://hgdownload.cse.ucsc.edu/goldenPath/hg19/bigZips/) with the burrows-wheeler alignment tool (BWA v.0.7.5a) and duplicate reads were marked (Picard-tools 1.56) (http://picard.sourceforge.net/). Next, we used the Genome-Analysis-Toolkit according to the recommended protocols for variant recalibration (v. 2.8-1-g932cd3a) and variant calling (v.3.3-0-g37228af) as described in the respective best-practices guidelines (https://www.broadinstitute.org/gatk/guide/best-practices), as described in DePristo, M. A., et al.: A framework for variation discovery and genotyping using next-generation DNA sequencing data, Nat Genet, 2011, 43(5): p. 491-8.

### Normalization and Removal of Technical Variations and Batch Effects

To remove unwanted technical variation, we applied a modified danes normalization procedure across all methylation measurements. Danes normalization is part of the wateRmelon package. The normalization procedure is based on between-array quantile normalization of methylated and unmethylated raw signal intensities of red and green channels together and thus also accounts for dye bias. However, between-array quantile normalization as initially developed for gene expression data is controversial for methylation data as overall methylation distributions may differ strongly between samples, tissues and diseases states. Consequently, we modified the danes normalization approach by not applying quantile normalization for between-array normalization but cyclicloess normalization instead. Cyclicloess normalization is similar in effect and intention to quantile normalization, but with the advantage that it does not as drastically normalize extreme cases and still preserves major distributional differences.

To account for batch effects, we performed duplicate measurements on different chips of in total 8 samples and used the duplicates for bridging the methylation-values of different analysis batches based on the duplicates only using the removeBatchEffect function from the limma package, as described in Ritchie, M. E., et al., limma powers differential expression analyses for RNA-sequencing and microarray studies, Nucleic Acids Res, 2015, 43(7): p. e47. Following batch bridging, duplicate measurements were averaged before downstream statistical analysis.

### Epigenome-Wide Association Analysis

Deregulated methylation sites were identified by linear modelling and moderated t-tests including age and gender using the limma package, as described above.

To also correct for potential genomic inflation in the discovery cohort, we performed principal component analysis on methylation measurements and identified principal components (PCs) which were associated with known confounders (e.g. technical such as analysis date and biological confounders) at FDR (false discovery rate)<=0.05. Again, deregulated methylation sites were subsequently identified by linear modelling and moderated t-tests including age and gender all identified PCs as covariates using the limma package. Statistical analyses were carried out in R-3.2.2. FDR correction of significance levels was performed using the Benjamini-Hochberg procedure.

### Transcriptome Analysis

RNAseq libraries were generated using TrueSeq RNA Sample Prep Kit (Illumina), and sequencing was performed 2×75 bp on a HiSeq2000 (Illumina) sequencer. Unstranded paired end raw read files were mapped with STAR v2.4.1c using GRCh37/hg19 and the Gencode 19 gene model (http://www.gencodegenes.org/). Only uniquely mapped reads were counted into genes using subread's featureCounts program (subread version 1.4.6.p1). Prior to statistical analyses, genes with very low expression levels (average reads <=1, detected reads in less than 50% of the samples) were removed. Count data was normalized by r log normalization as described in Love, M. I., W. Huber, and S. Anders: Moderated estimation of fold change and dispersion for RNA-seq data with DESeq2, Genome Biol, 2014, 15(12): p. 550, which is an improved method of the variance stabilization transformation as recommended for eQTL (expression quantitative trait loci) by the original MatrixEQTL publication of Shabalin, A. A.: Matrix eQTL: ultra fast eQTL analysis via large matrix operations. Bioinformatics, 2012. 28(10): p. 1353-8.

### Epigenome-Transcriptome Association Analysis

An eQTL analysis between methylation sites and gene expressions was performed on 34 DCM patients and 25 controls for which high quality transcriptome data from biopsy samples could be obtained (out of the total of 38 DCM patients and 31 controls which were profiled on the methylation level). MatrixEQTL and linear models were used to correlate the expression profiles of 19,418 genes with the 311,222 methylation sites in a range of 10.000 bp up- and downstream of the genes as well as in the gene body region out of the 394,247 that passed quality control. Association with the RNA expression level was carried out using the myocard samples.

### Epigenetic Region of Interest Definition

DNA methylation of the gene body as well as adjacent non-coding regulatory regions is known to be an important regulation mechanism for gene expression. For aggregated analyses on region level, aggregate significance level was then obtained using the simes procedure for all methylation loci as the simes procedure has been shown to generally perform well, also for correlated significance levels, as described in RØDLAND, E. A.: Simes' procedure is ‘valid on average’, Biometrika, 93: p. 742-746. To determine the distance for significant associations between DNA methylation and RNA expression, an aggregate significance level for associations was obtained using the simes procedure for all methylation loci within the gene body and adjacent regions at increased distances, as the simes procedure has been shown to generally perform well as an aggregate measure for significant associations, also for correlated significance levels. The results thereof are shown in FIG. 4, with SL being the Simes significance level and D the distance for association between DNA methylation and gene expression at increasing distances.

As shown in FIG. 4, the simes measure (−log 10 simes significance level) only starts to drop significantly when increasing the distance from 10.000 to 100.000 bp as until 10.000 bp the difference from 0 bp distance is less than one standard deviation (horizontal lines in the figure, as estimated by 10-fold random sampling with replacement to estimate the standard deviation). As a result, a cut-off was chosen at a distance of 10.000 bp.

### Epigenetic and Transcriptomic Marker Definition

From the discovery cohort first four different categories of biomarkers (Cat. 1-4) were identified which show concordant dysregulation in methylation profiles in DCM either across molecular levels (i.e. epigenetic and transcriptomic; Cat. 4), tissues (i.e. cardiac tissue and blood; Cat. 2 and 3) or even both (Cat. 1).

The following categories (Cat. 1-4) describe molecular marker of HF and DCM.

Cat. 1a describes genomic regions that show coordinated hyper/hypo methylation in HF/DCM in peripheral blood and myocardial tissue and are associated with mRNA expression levels of genes of cardiac relevance in the myocard which are deregulated in HF/DCM. The genes are given in Table 12.

TABLE 12
Data for Cat. 1a

| ID | CHR | POS | GENE NAME | CARDIAC RELEVANCE |
|---|---|---|---|---|
| cg03649649 | 17 | 56408197 | MIR142 | Required for Survival Signalling |
| | | | (BZRAP1-AS1) | During Adaptive Hypertrophy |
| cg06613515 | 15 | 77287656 | PSTPIP1 | Immune System (Arthritis) |
| cg10495227 | 16 | 82970452 | CDH13 | Cadherin 13 (Heart) |
| cg02856109 | 2 | 80531656 | CTNNA2 | Catenin (Cadherin-Associated |
| | | | (LRRTM1) | Protein), Alpha 2 |
| cg17033080 | 2 | 217508851 | IGFBP2 | Insuline-Like Growth Factor |
| | | | | Binding Protein 2 |
| cg20689294 | 10 | 129846082 | PTPRE | Regulates Insulin-induced Tyrosine |
| | | | | phosphorylation of Insulin |
| | | | | Receptor |

Cat. 1b describes genomic regions that show coordinated hyper/hypo methylation in HF/DCM in peripheral blood and myocardial tissue and are associated with mRNA expression levels of genes of unknown cardiac relevance in the myocard which are deregulated in HF/DCM. The genes are given in Table 13.

TABLE 13
Data for Cat. 1b

| ID | CHR | POS | GENE NAME |
|---|---|---|---|
| cg20720059 | 2 | 14772731 | FAM84A |
| cg16362232 | 11 | 430036 | ANO9 |

Cat. 2 describes genomic regions that show coordinated hyper/hypo methylation in HF/DCM in peripheral blood and myocardial tissue and cluster in chromosome bands with heart specific genes. The genes are given in Table 14.

TABLE 14
Data for Cat. 2

| | | | | CHROMOSOME |
|---|---|---|---|---|
| ID | CHR | POS | GENE NAME | BAND |
| cg05532869 | 11 | 2368070 | CD81-AS1 | Chr11p15.5 |
| cg12121166 | 11 | 2376275 | CD81-AS1 | (Cat2a) |
| cg20751395 | 11 | 2594153 | KCNQ1 | |
| cg13145504 | 11 | 2594840 | KCNQ1 | |
| cg22239603 | 11 | 2690304 | KCNQ1 | |
| cg21522797 | 16 | 81806083 | PLCG2 | Chr16q23.3 |
| cg02516845 | 16 | 84076320 | SLC38A8 | (Cat2b) |
| cg10495227 | 16 | 82970452 | CDH13 | |
| cg25794153 | 18 | 31805151 | NOL4 | Chr18q12.1 |
| cg26530706 | 18 | 32173093 | DTNA | (Cat2b) |
| cg22648949 | 18 | 30351983 | KLHL14 | |

Cat. 3 describes genomic regions that show coordinated hyper/hypo methylation in HF/DCM in peripheral blood and myocardial tissue but do not fall within Cat. 1 or 2. Two sub-categories were identified.

Cat. 3a is related to genomic regions in genes with cardiac relevance. The genes are given in Table 15.

TABLE 15
Data for Cat. 3a

| ID | CHR | POS | GENE NAME |
|---|---|---|---|
| cg24068761 | 1 | 6146988 | KCNAB2 |
| cg12748607 | 2 | 40678691 | SLC8A1 |
| cg09283977 | 2 | 240082420 | HDAC4 |
| cg03912954 | 9 | 140773129 | CACNA1B |
| cg01744056 | 11 | 12524208 | PARVA |
| cg16932472 | 13 | 98605951 | IPO5 |
| cg14287235 | 14 | 24804339 | RIPK3 |
| cg25076767 | 14 | 24836148 | NFATC4 |
| cg03368634 | 16 | 3824553 | CREBBP |
| cg03547745 | 17 | 70117522 | SOX9 |
| cg09306675 | 20 | 45523996 | EYA2 |
| cg26943378 | 22 | 50689804 | HDAC10 |

Cat. 3b is related to genomic regions in genes with unknown cardiac relevance. The genes are given in Table 16.

TABLE 16
Data for Cat. 3b

| ID | CHR | POS | GENE NAME |
|---|---|---|---|
| cg05536984 | 1 | 32083535 | HCRTR1 |
| cg15061530 | 1 | 32827834 | TSSK3 |
| cg12431729 | 1 | 41827960 | FOXO6 |
| cg11750103 | 1 | 53238307 | ZYG11B |
| cg26963271 | 1 | 66259081 | PDE4B |
| cg00791468 | 1 | 111148984 | KCNA2 |
| cg13072446 | 1 | 151019727 | C1orf56 |
| cg13474719 | 1 | 177034184 | ASTN1 |
| cg23548885 | 2 | 47150 | FAM110C |
| cg26659079 | 2 | 5836181 | SOX11 |
| cg05939149 | 2 | 43986106 | PLEKHH2 |
| cg18809126 | 3 | 11623526 | VGLL4 |
| cg24823485 | 3 | 42626083 | SS18L2 |
| cg06327727 | 3 | 62354546 | PTPRG-AS1 |
| cg27338287 | 3 | 190580644 | GMNC |
| cg08923494 | 4 | 166797526 | TLL1 |
| cg14553364 | 5 | 137674194 | FAM53C |
| cg12364324 | 5 | 170848039 | FGF18 |
| cg26651429 | 5 | 171469429 | STK10 |
| cg13898548 | 5 | 176924827 | PDLIM7 |
| cg05560494 | 6 | 33241974 | RPS18 |
| cg15089846 | 6 | 75798778 | COL12A1 |
| cg26732340 | 6 | 157342220 | ARID1B |
| cg00155447 | 7 | 99627985 | ZKSCAN1 |
| cg08832906 | 7 | 139208852 | CLEC2L |
| cg00461149 | 7 | 157452656 | PTPRN2 |
| cg09121695 | 8 | 37655503 | GPR124 |
| cg09125812 | 8 | 41625127 | ANK1 |
| cg16587988 | 8 | 42948547 | POMK |
| cg16491617 | 8 | 54164391 | OPRK1 |
| cg01924448 | 9 | 14313043 | NFIB |
| cg24701032 | 10 | 17183411 | TRDMT1 |
| cg17003301 | 10 | 43048646 | ZNF37BP |
| cg25497250 | 10 | 95326974 | FFAR4 |
| cg26554592 | 10 | 108924398 | SORCS1 |
| cg12486121 | 11 | 7535256 | PPFIBP2 |
| cg19279432 | 11 | 75110505 | RPS3 |
| cg14727452 | 11 | 117283767 | CEP164 |
| cg07732097 | 12 | 49330158 | ARF3 |
| cg13222752 | 14 | 74892569 | SYNDIG1L |
| cg05295297 | 14 | 85999731 | FLRT2 |
| cg14400498 | 14 | 85999933 | FLRT2 |
| cg21883598 | 15 | 45404157 | DUOX2 |
| cg22381808 | 15 | 69452537 | GLCE |
| cg11611600 | 15 | 84323154 | ADAMTSL3 |
| cg01852244 | 16 | 47494711 | PHKB |
| cg07665510 | 17 | 55952063 | CUEDC1 |
| cg14787267 | 17 | 77951858 | TBC1D16 |
| cg14893129 | 17 | 78152051 | CARD14 |
| cg24498538 | 18 | 5295760 | ZBTB14 |
| cg24362812 | 18 | 11148769 | PIEZO2 |
| cg12113740 | 18 | 52625368 | CCDC68 |
| cg03124313 | 19 | 36036028 | TMEM147 |
| cg09430060 | 19 | 49306842 | BCAT2 |
| cg19098268 | 19 | 57352269 | PEG3 |
| cg08448665 | 21 | 40984780 | B3GALT5 |
| cg01777170 | 22 | 38337780 | MICALL1 |

Cat. 4 describes genomic regions that show correlated, deregulated methylation and mRNA expression patterns in HF/DCM in the myocardial tissue. The genes are given in Table 17.

TABLE 17
Data for Cat. 4

| Gene | Chr | Start | End | Width | Strand |
|---|---|---|---|---|---|
| PIK3CD | 1 | 9710791 | 9790172 | 77382 | + |
| THEMIS2 | 1 | 28198056 | 28214196 | 14141 | + |
| PTPRC | 1 | 198606802 | 198727545 | 118744 | + |
| PLEK | 2 | 68591306 | 68625585 | 32280 | + |
| ARL4C | 2 | 235400686 | 235406697 | 4012 | − |
| SPI1 | 11 | 47375412 | 47401127 | 23716 | − |
| FERMT3 | 11 | 63973151 | 63992354 | 17204 | + |
| LCP1 | 13 | 46699056 | 46787006 | 85951 | − |
| ISG20 | 15 | 89178385 | 89200714 | 20330 | + |
| IL4R | 16 | 27323990 | 27377099 | 51110 | + |
| CORO1A | 16 | 30193149 | 30201397 | 6249 | + |
| ITGAM | 16 | 31270312 | 31345213 | 72902 | + |
| COTL1 | 16 | 84598201 | 84652683 | 52483 | − |
| IRF8 | 16 | 85931410 | 85957215 | 23806 | + |
| ACAP1 | 17 | 7238849 | 7255797 | 14949 | + |
| TMC8 | 17 | 76125852 | 76140049 | 12198 | + |
| ICAM1 | 19 | 10380512 | 10398291 | 15780 | + |
| TYROBP | 19 | 36394304 | 36400197 | 3894 | − |
| NKG7 | 19 | 51873861 | 51876969 | 1109 | − |
| MAFB | 20 | 39313489 | 39318880 | 3392 | − |
| ITGB2 | 21 | 46304869 | 46352904 | 46036 | − |
| PARVG | 22 | 44567837 | 44616413 | 46577 | + |

Further, the following categories (Ca. 5-7) describe molecular marker of HF and DCM that were further identified.

Cat. 5 describes genomic regions that show coordinated hyper/hypo methylation in HF/DCM in peripheral blood and myocardial tissue and are associated with mRNA expression levels in the myocard. The genes are given in Table 18.

TABLE 18
Data for Cat. 5

| ID | Ensemble ID | Gene name | Chr | Pos |
|---|---|---|---|---|
| cg25943276 | ENSG00000182667 | NTM | 11 | 131533284 |
| cg24884140 | ENSG00000108641 | B9D1 | 17 | 19250190 |
| cg12115081 | ENSG00000170390 | DCLK2 | 4 | 151038391 |

Cat. 6 describes genomic regions that show coordinated methylation and gene expression changes in HF/DCM in the myocardial tissue and are also associated with HF/DCM on gene level. The genes are given in Table 19.

TABLE 19
Data for Cat. 6

| ID | Ensemble ID | Gene name | Chr | Pos |
|---|---|---|---|---|
| cg24720355 | ENSG00000092607 | TBX15 | 1 | 119526255 |
| cg24144440 | ENSG00000092607 | TBX15 | 1 | 119526882 |
| cg02829688 | ENSG00000092607 | TBX15 | 1 | 119527008 |
| cg21647227 | ENSG00000092607 | TBX15 | 1 | 119527111 |
| cg05940231 | ENSG00000092607 | TBX15 | 1 | 119532189 |
| cg08942939 | ENSG00000092607 | TBX15 | 1 | 119532542 |
| cg21301805 | ENSG00000092607 | TBX15 | 1 | 119534644 |
| cg10587082 | ENSG00000076356 | PLXNA2 | 1 | 208293478 |
| cg01876531 | ENSG00000076356 | PLXNA2 | 1 | 208405868 |
| cg16045271 | ENSG00000076356 | PLXNA2 | 1 | 208412585 |
| cg04685570 | ENSG00000255399 | TBX5-AS1 | 12 | 114841202 |
| cg00182639 | ENSG00000255399 | TBX5-AS1 | 12 | 114841671 |
| cg00642359 | ENSG00000255399 | TBX5-AS1 | 12 | 114841708 |
| cg22045225 | ENSG00000255399 | TBX5-AS1 | 12 | 114841792 |
| cg21907579 | ENSG00000255399 | TBX5-AS1 | 12 | 114845868 |
| cg03877376 | ENSG00000255399 | TBX5-AS1 | 12 | 114846162 |
| cg03877376 | ENSG00000089225 | TBX5 | 12 | 114846162 |
| cg17645823 | ENSG00000255399 | TBX5-AS1 | 12 | 114846321 |
| cg17645823 | ENSG00000089225 | TBX5 | 12 | 114846321 |
| cg10281002 | ENSG00000255399 | TBX5-AS1 | 12 | 114846399 |
| cg10281002 | ENSG00000089225 | TBX5 | 12 | 114846399 |
| cg16458436 | ENSG00000255399 | TBX5-AS1 | 12 | 114846412 |
| cg16056219 | ENSG00000119681 | LTBP2 | 14 | 75043777 |
| cg14340889 | ENSG00000119681 | LTBP2 | 14 | 75072120 |
| cg08140459 | ENSG00000119681 | LTBP2 | 14 | 75086513 |
| cg09004195 | ENSG00000116106 | EPHA4 | 2 | 222323493 |
| cg13364311 | ENSG00000116106 | EPHA4 | 2 | 222333289 |
| cg03850035 | ENSG00000116106 | EPHA4 | 2 | 222367110 |

Cat. 7 describes genomic regions that show coordinated methylation and gene expression changes in HF/DCM in the myocardial tissue. The genes are given in Table 20.

TABLE 20
Data for Cat. 7

| ID | Ensemble ID | Name | Chr | Pos |
|---|---|---|---|---|
| cg01179095 | ENSG00000175206 | NPPA | 1 | 11900652 |
| cg03603260 | ENSG00000197622 | CDC42SE1 | 1 | 151021364 |
| cg13740187 | ENSG00000143549 | TPM3 | 1 | 154164699 |
| cg14529268 | ENSG00000183888 | C1orf64 | 1 | 16335452 |
| cg09013655 | ENSG00000198756 | COLGALT2 | 1 | 184005063 |
| cg01963906 | ENSG00000142765 | SYTL1 | 1 | 27677240 |
| cg16254946 | ENSG00000174332 | GLIS1 | 1 | 54058616 |
| cg08029603 | ENSG00000223764 | | 1 | 854824 |
| cg09608533 | ENSG00000121898 | CPXM2 | 10 | 125618188 |
| cg04109883 | ENSG00000165633 | VSTM4 | 10 | 50289110 |
| cg00857536 | ENSG00000165633 | VSTM4 | 10 | 50298306 |
| cg24699895 | ENSG00000156515 | HK1 | 10 | 71094286 |
| cg02378006 | ENSG00000107731 | UNC5B | 10 | 73026288 |
| cg07216529 | ENSG00000138134 | STAMBPL1 | 10 | 90712739 |
| cg06595154 | ENSG00000072952 | MRVI1 | 11 | 10716164 |
| cg11822932 | ENSG00000135363 | LMO2 | 11 | 33913716 |
| cg02337873 | ENSG00000175602 | CCDC85B | 11 | 65659393 |
| cg21746120 | ENSG00000162337 | LRP5 | 11 | 68142234 |
| cg08679180 | ENSG00000110237 | ARHGEF17 | 11 | 73034459 |
| cg10630085 | ENSG00000054965 | FAM168A | 11 | 73108402 |
| cg15542639 | ENSG00000110218 | PANX1 | 11 | 93885254 |
| cg20735050 | ENSG00000166025 | AMOTL1 | 11 | 94521117 |
| cg24088496 | ENSG00000184384 | MAML2 | 11 | 96071506 |
| cg11513088 | ENSG00000123094 | RASSF8 | 12 | 26111821 |
| cg22070156 | ENSG00000198542 | ITGBL1 | 13 | 102104991 |
| cg07403350 | ENSG00000139826 | ABHD13 | 13 | 108867111 |
| cg02215357 | ENSG00000139675 | HNRNPA1L2 | 13 | 53191046 |
| cg19910802 | ENSG00000227051 | C14orf132 | 14 | 96520233 |
| cg27370471 | ENSG00000140479 | PCSK6 | 15 | 101932559 |
| cg05377733 | ENSG00000137809 | ITGA11 | 15 | 68645969 |
| cg17258195 | ENSG00000129009 | ISLR | 15 | 74466337 |
| cg27009545 | ENSG00000136404 | TM6SF1 | 15 | 83776915 |
| cg09284275 | ENSG00000133392 | MYH11 | 16 | 15923487 |
| cg04674421 | ENSG00000169181 | GSG1L | 16 | 28079611 |
| cg09509739 | ENSG00000262766 | | 16 | 31129199 |
| cg02696327 | ENSG00000102924 | CBLN1 | 16 | 49312543 |
| cg27232494 | ENSG00000175662 | TOM1L2 | 17 | 17832220 |
| cg26535547 | ENSG00000161654 | LSM12 | 17 | 42151680 |
| cg03995300 | ENSG00000129204 | USP6 | 17 | 5019989 |
| cg00864012 | ENSG00000136478 | TEX2 | 17 | 62294665 |
| cg06331359 | ENSG00000181045 | SLC26A11 | 17 | 78190755 |
| cg12475142 | ENSG00000226137 | BAIAP2-AS1 | 17 | 79012396 |
| cg22588546 | ENSG00000133026 | MYH10 | 17 | 8382941 |
| cg01085362 | ENSG00000121297 | TSHZ3 | 19 | 31848310 |
| cg09779027 | ENSG00000171105 | INSR | 19 | 7224513 |
| cg00428638 | ENSG00000171105 | INSR | 19 | 7224713 |
| cg07077013 | ENSG00000128652 | HOXD3 | 2 | 177025198 |
| cg10035294 | ENSG00000135903 | PAX3 | 2 | 223164925 |
| cg17245125 | ENSG00000119771 | KLHL29 | 2 | 23843711 |
| cg05403316 | ENSG00000115310 | RTN4 | 2 | 55339939 |
| cg16665041 | ENSG00000215251 | FASTKD5 | 20 | 3148787 |
| cg22164891 | ENSG00000171940 | ZNF217 | 20 | 52199729 |
| cg20979153 | ENSG00000171940 | ZNF217 | 20 | 52199748 |
| cg21172011 | ENSG00000159216 | RUNX1 | 21 | 36577638 |
| cg14703829 | ENSG00000099910 | KLHL22 | 22 | 20780298 |
| cg01640635 | ENSG00000100196 | KDELR3 | 22 | 38864868 |
| cg13066481 | ENSG00000065534 | MYLK | 3 | 123372199 |
| cg18274619 | ENSG00000074416 | MGLL | 3 | 127494852 |
| cg20950633 | ENSG00000206561 | COLQ | 3 | 15540137 |
| cg00434119 | ENSG00000058866 | DGKG | 3 | 186080868 |
| cg10960375 | ENSG00000114853 | ZBTB47 | 3 | 42694144 |
| cg02316506 | ENSG00000114853 | ZBTB47 | 3 | 42694803 |
| cg24074783 | ENSG00000163788 | SNRK | 3 | 43405624 |
| cg08052292 | ENSG00000163947 | ARHGEF3 | 3 | 56789178 |
| cg09427605 | ENSG00000151612 | ZNF827 | 4 | 146740968 |
| cg19116959 | ENSG00000151612 | ZNF827 | 4 | 146841472 |
| cg25924602 | ENSG00000163145 | C1QTNF7 | 4 | 15397288 |
| cg13832772 | ENSG00000109771 | LRP2BP | 4 | 186283800 |
| cg23664174 | ENSG00000072201 | LNX1 | 4 | 54357316 |
| cg14855841 | ENSG00000169248 | CXCL11 | 4 | 76945459 |
| cg21631086 | ENSG00000228672 | PROB1 | 5 | 138718914 |
| cg11462252 | ENSG00000184347 | SLIT3 | 5 | 168139607 |
| cg13112511 | ENSG00000113448 | PDE4D | 5 | 58882753 |
| cg02511723 | ENSG00000131711 | MAP1B | 5 | 71402031 |
| cg25515801 | ENSG00000231500 | RPS18 | 6 | 33240333 |
| cg04201373 | ENSG00000030110 | BAK1 | 6 | 33551533 |
| cg00604356 | ENSG00000105851 | PIK3CG | 7 | 106507474 |
| cg09374838 | ENSG00000204934 | ATP6V0E2- | 7 | 149578384 |
| | | AS1 | | |
| cg09374838 | ENSG00000171130 | ATP6V0E2 | 7 | 149578384 |
| cg26672672 | ENSG00000136205 | TNS3 | 7 | 47479433 |
| cg03143486 | ENSG00000164818 | HEATR2 | 7 | 811491 |
| cg11201447 | ENSG00000249859 | PVT1 | 8 | 128808063 |
| cg25079691 | ENSG00000221818 | EBF2 | 8 | 25908057 |
| cg04244354 | ENSG00000221818 | EBF2 | 8 | 25908279 |
| cg12563372 | ENSG00000221818 | EBF2 | 8 | 25908503 |
| cg14523204 | ENSG00000138835 | RGS3 | 9 | 116359818 |

### Example 2

Methods and Results (summary): Infinium HumanMethylation450 was used for high-density epigenome wide mapping of DNA methylation in left ventricular biopsies and whole peripheral blood of living probands. RNA deep sequencing was performed on the same samples in parallel. Whole genome sequencing of all patients allowed exclusion of promiscuous genotype-induced methylation calls. In the screening stage, we detected 59 epigenetic loci that are significantly associated with DCM (FDR corrected p≤0.05), with three of them reaching epigenome-wide significance at p≤5×10-8. Twenty-seven (46%) of these loci could be replicated in independent cohorts, underlining the role of epigenetic regulation of key cardiac transcription regulators. Using a staged multi-omics study design, we link a subset of 517 epigenetic loci with DCM and cardiac gene expression. Furthermore, we identified distinct epigenetic methylation patterns that are conserved across tissues, rendering these CpGs novel epigenetic biomarkers for heart failure.

### Material and Methods

### Patient Enrolment and Study Design

Patient inclusion for the present study was approved by the ethics committee, medical faculty of Heidelberg University. All participants have given written informed consent to allow for molecular analysis of blood and left-over tissue. The diagnosis of Dilated Cardiomyopathy (DCM) was confirmed after excluding coronary artery disease (CAD) as determined by coronary angiography, valvular heart disease was excluded by cMRI and echocardiography and myocarditis/inflammatory DCM by histopathology (Richardson P, et al., Report of the 1995 World Health Organization/International Society and Federation of Cardiology Task Force on the Definition and Classification of cardiomyopathies. Circulation. 1996; 93:841-2). Patients with history of uncontrolled hypertension, myocarditis, regular alcohol consumption, illicit drugs or cardio-toxic chemotherapy were also excluded. To include the clinical continuum of systolic heart failure, also early but symptomatic disease stages (LV-EF between >45 and <55%) were included.

After screening of n=135 DCM patients, n=41 met all inclusion and no exclusion criteria and had sufficient amounts of left-over LV ventricular biopsies (LV free wall) and peripheral blood samples available for the laborious high-throughput analyses of DNA methylation, genome- and mRNA sequencing. Control LV-biopsy specimens were obtained from stable and symptom-free patients after heart transplantation (n=31; HTX was at least 6 months ago), who had normal systolic and diastolic function and no evidence for relevant vasculopathy or acute/chronic organ rejection as judged by coronary angiography and immuno-histopathology. Controls for whole blood samples (n=31) had a cardiovascular risk profile (Hypertension, Hyperlipidemia), but completely normal systolic and diastolic left ventricular function without evidence for heart failure or significant (>50%) coronary artery disease.

As an independent validation cohort, left ventricular myocardium of n=18 DCM patients and n=8 previously healthy road accident victims were included. The independent validation cohort for peripheral blood consisted of n=9 DCM patients and n=28 clinical controls. A third replication cohort for top blood-based markers included n=82 DCM patients (Institute for Cardiomyopathies Heidelberg) and n=109 Controls (Noko/normal control project).

### Biomaterial Processing

Biopsy specimens were obtained from the apical part of the free left ventricular wall (LV) from DCM patients or cardiac transplant patients (controls) undergoing cardiac catheterization using a standardized protocol. Biopsies were immediately washed in ice-cold saline (0.9% NaCl) and transferred and stored in liquid nitrogen until DNA and RNA was extracted. After diagnostic workup of the biopsies (histopathology), remaining material was evenly dissected to isolate DNA and RNA. DNA was extracted from blood with DNA Blood Maxi Kit (Qiagen) and from biopsies with Allprep Kit (Qiagen). Total RNA was extracted using the miRNeasy mini Kit (blood) or Allprep Kit (biopsies) according to the manufacturer's protocol (Qiagen, Germany) from biopsies and peripheral blood. RNA purity and concentration were determined using the Bioanalyzer 2100 (Agilent Technologies, Berkshire, UK) with a Eukaryote Total RNA Pico assay for RNA from biopsies and with Eukaryote Total RNA Nano assay for RNA from blood.

### DNA Methylation Profiling, RNA and Whole-Genome Sequencing

Methylation profiles were measured using the Illumina 450 k methylation assay, following procedures as described earlier (Bibikova M, et al., High density DNA methylation array with single CpG site resolution. Genomics. 2011; 98:288-95). From each patient, we subjected 200 ng DNA (blood and biopsy) for the measurements. Methylation sites with a detection p-value of >0.05 in more than 10% of the samples were removed from analysis. Methylation levels with a detection p-value of >0.05 in less than 10% of the samples were imputed via knn-imputation (Hastie T T, R, Narasimhan, B Chu, G. impute: impute: Imputation for microarray data. R package version 1460. 2016). To reduce the effects of genomic variation on methylation measurements, we excluded methylation sites that were potentially influenced by genotypes present in more than 10% of the DCM patients and that lie within the 50 bp probe region as assessed by whole-genome sequencing. Methylation levels with variants in less than 10% of the DCM patients were imputed. We further removed all probes on X and Y chromosomes as well as probes that have been identified by Chen et al. to cross-hybridize with non-targeted DNA (Chen Y A, et al., Discovery of cross-reactive probes and polymorphic CpGs in the Illumina Infinium HumanMethylation450 microarray. Epigenetics. 2013; 8:203-9). Finally, 394,247 methylation sites passed QC.

DNA methylation was validated for the top two biomarker candidate loci by the MassARRAY technique as previously described (Haas J, et al., Alterations in cardiac DNA methylation in human dilated cardiomyopathy. EMBO Mol Med. 2013; 5:413-29). Briefly, 400 ng genomic DNA was chemically modified with sodium bisulfite. The bisulfite-treated DNA was PCR-amplified by primers designed to cover the Infinium probes cg06688621 and cg01642653 (cg06688621 primer sequences GGTGTTTTTTGTTTAGTATTTTTTAGAG and AGGGTAGATTTGAGGTAGTTTAGGA; cg01642653 primer sequences TAGGTGTTTTTTAGGGTTGTTTTTT and GTTGGGGAATTTGTTGTTTATTAG). The amplicons were transcribed by T7 polymerase, followed by T-specific-RNAase-A cleavage. The digested fragments were quantified by MALDI-TOF-based technique (MassARRAY).

1 μg of total peripheral blood gDNA was sheared using the Covaris™ 5220 system, applying 2 treatments of 60 seconds each (peak power=140; duty factor=10) with 200 cycles/burst. 500 ng of sheared gDNA was taken and whole genome libraries were prepared using TruSeq DNA sample preparation kit according to manufacturer's protocols (Illumina, San Diego, US). Sequencing was performed on an Illumina HiSeq 2000, using TruSeq SBS Kit v3 and reading two times 100 bp for paired end sequencing, on four lanes of a sequencing flowcell.

Demultiplexing of the raw sequencing reads and generation of the fastq files was done using CASAVA v.1.82. The raw reads were then mapped to the human reference genome (GRCh37/hg19) with the burrows-wheeler alignment tool (BWA v.0.7.5a) (Li H and Durbin R. Fast and accurate short read alignment with Burrows-Wheeler transform. Bioinformatics. 2009; 25:1754-60) and duplicate reads were marked (Picard-tools 1.56) (http://picard.sourceforge.net/). Next, we used the Genome-Analysis-Toolkit according to the recommended protocols for variant recalibration (v. 2.8-1-g932cd3a) and variant calling (v.3.3-0-g37228af) as described in the respective best-practices guidelines (https://www.broadinstitute.org/gatk/guide/best-practices) (DePristo M A, et al., A framework for variation discovery and genotyping using next-generation DNA sequencing data. Nature genetics. 2011; 43:491-8).

### Statistical Analysis

Regarding detailed information on normalization and removal of technical and batch effects, association statistics, overrepresentation and gene ontology analyses, the following is applied.

### Normalization and Removal of Technical Variations and Batch Effects

To remove unwanted technical variation, we applied a modified danes normalization procedure across all methylation measurements. Danes normalization is part of the wateRmelon package and was first described by Pidsley (Pidsley R, et al., A data-driven approach to preprocessing Illumina 450K methylation array data. BMC Genomics. 2013; 14:293). The normalization procedure is based on between-array quantile normalization of methylated and unmethylated raw signal intensities of red and green channels together and thus accounts for dye bias. However, between-array quantile normalization as initially developed for gene expression data is controversial for methylation data as overall methylation distributions may differ strongly between samples, tissues and diseases states. Consequently, we modified the danes normalization approach by not applying quantile normalization for between-array normalization but cyclicloess normalization instead. Cyclicloess normalization is similar in effect and intention to quantile normalization, but with the advantage that it does not as drastically normalize extreme cases and still preserves major distributional differences (Ballman K V, Grill D E, Oberg A L and Therneau T M. Faster cyclic loess: normalizing RNA arrays via linear models. Bioinformatics. 2004; 20:2778-86).

All samples were measured in 5 different batches and each batch contained duplicate samples from other batches. To remove technical variation possibly introduced by the measurement batch, the duplicate measurements of in total 8 samples were used for bridging the methylation-values (Du P, et al., Comparison of Beta-value and M-value methods for quantifying methylation levels by microarray analysis. BMC Bioinformatics. 2010; 11:587) of different analysis batches using the removeBatchEffect function from the limma package (Ritchie M E, Phipson B, Wu D, Hu Y, Law C W, Shi W and Smyth G K. limma powers differential expression analyses for RNA-sequencing and microarray studies. Nucleic Acids Res. 2015; 43:e47). Following batch bridging, duplicate measurements were averaged before downstream statistical analysis.

### Epigenome-Wide Association Analysis

To correct for genomic inflation in the discovery cohort, we performed principal component analysis on methylation measurements and identified principal components (PCs), which were associated with known confounders (e.g. technical such as analysis date and biological confounders such as medication) at FDR≤0.05, see Tables 21 and 22.

TABLE 21
Confounders for methylation measurements from myocardial tissue in the discovery
cohort that are associated with principal components after FDR correction. PC1-4
and 6-7 were subsequently used for correction of potential genomic inflation.

| | Explained | Cum. Explained | Measurement | Medication | |
|---|---|---|---|---|---|
| PC | Variation | Variation | Batch | Tacrolimus | Mycophenolat |
| 1 | 0.11603 | 0.11603 | 1.17E−07 | 0.87787399 | 0.1433775 |
| 2 | 0.11056 | 0.22659 | 0.004955317 | 0.94099466 | 0.94099466 |
| 3 | 0.08126 | 0.30784 | 0.000119371 | 0.48469009 | 0.45229374 |
| 4 | 0.05514 | 0.36298 | 6.53E−09 | 0.00591254 | 0.00195786 |
| 5 | 0.03721 | 0.40019 | 0.23171337 | 0.6788642 | 0.51621221 |
| 6 | 0.02961 | 0.4298 | 0.014540305 | 0.91277464 | 0.88088841 |
| 7 | 0.02114 | 0.45094 | 0.485198384 | 0.02555192 | 0.05004367 |
| 8 | 0.01917 | 0.47012 | 0.346068453 | 0.9661979 | 0.9661979 |
| 9 | 0.01637 | 0.48648 | 0.573861536 | 0.90992672 | 0.87682897 |
| 10 | 0.01602 | 0.5025 | 0.431079505 | 0.84476548 | 0.74247531 |

TABLE 22
Known confounders for methylation measurements from peripheral blood in
the discovery cohort that was identified to be significantly associated
with principal components after FDR correction. PC1-4 as well as age and
gender were subsequently included for correction of genomic inflation.

| | | Cum. | | | | |
|---|---|---|---|---|---|---|
| | Explained | Explained | Measurement | | | |
| PC | Variation | Variation | Batch | Weight | BMI | Age |
| 1 | 0.17702 | 0.17702 | 3.65E−08 | 0.74142811 | 0.82657779 | 0.88017013 |
| 2 | 0.074 | 0.25102 | 0.17882175 | 0.00432245 | 0.00881378 | 0.7547449 |
| 3 | 0.05376 | 0.30478 | 1.88E−09 | 0.99029277 | 0.99029277 | 0.08324938 |
| 4 | 0.03977 | 0.34455 | 4.84E−06 | 0.95067972 | 0.95183356 | 0.76970735 |
| 5 | 0.02545 | 0.37001 | 0.89104493 | 0.90205601 | 0.89104493 | 7.34E−05 |
| 6 | 0.01911 | 0.38912 | 0.1419809 | 0.98514199 | 0.98514199 | 0.97446499 |
| 7 | 0.01776 | 0.40688 | 0.74935875 | 0.74935875 | 0.74935875 | 0.74935875 |
| 8 | 0.0155 | 0.42238 | 0.83285365 | 0.84157735 | 0.84157735 | 0.84157735 |
| 9 | 0.01495 | 0.43732 | 0.74629486 | 0.08897591 | 0.27253148 | 0.74629486 |
| 10 | 0.01449 | 0.45182 | 0.90693645 | 0.90693645 | 0.93956553 | 0.3294711 |

Deregulated methylation sites were identified by linear modelling and moderated t-tests including age and gender as well as all identified PCs as covariates using the limma package (Ritchie M E, Phipson B, Wu D, Hu Y, Law C W, Shi W and Smyth G K. limma powers differential expression analyses for RNA-sequencing and microarray studies. Nucleic Acids Res. 2015; 43:e47). Methylation sites were subsequently directionally verified in verification cohorts including gender (as age was not available for all samples) as covariates. Statistical analyses were carried out in R-3.2.2 (R: A Language and Environment for Statistical Computing [computer program]. 2008). FDR correction of significance levels was performed using the Benjamini-Hochberg procedure (Benjamini Y and Hochberg Y. Controlling the False Discovery Rate—a Practical and Powerful Approach to Multiple Testing. J Roy Stat Soc B Met. 1995; 57:289-300). Significance levels from discovery and verification cohorts were combined using Fisher's method to combine results from independent tests.

### Transcriptome Analysis

RNA sequencing libraries were generated using TrueSeq RNA Sample Prep Kit (Illumina) and sequencing was performed 2×75 bp on a HiSeq2000 (Illumina) sequencer. Samples were sequenced to a median paired-end read count of 29.85 million. Unstranded paired-end raw read files were mapped with STAR v2.4.1c (Dobin A and Gingeras T R. Mapping RNA-seq Reads with STAR. Curr Protoc Bioinformatics. 2015; 51:11 14 1-11 14 19) using GRCh37/hg19 and the Gencode 19 gene model (http://www.gencodegenes.org/). Only uniquely mapped reads were counted into genes using subread's feature counts program (Liao Y, Smyth G K and Shi W. featureCounts: an efficient general purpose program for assigning sequence reads to genomic features. Bioinformatics. 2014; 30:923-30) (subread version 1.4.6.p1) and mapping percentages were median 88.08. Prior to statistical analyses, genes with very low expression levels (average reads <=1, detected reads in less than 50% of the samples) were removed. Count data was normalized by r log normalization (Love M I, Huber W and Anders S. Moderated estimation of fold change and dispersion for RNA-seq data with DESeq2. Genome Biol. 2014; 15:550), which is an improved method of the variance stabilization transformation (Anders S and Huber W. Differential expression analysis for sequence count data. Genome Biol. 2010; 11:R106) as recommended for eQTL by the original MatrixEQTL publication (Shabalin A A. Matrix eQTL: ultra fast eQTL analysis via large matrix operations. Bioinformatics. 2012; 28:1353-8).

### Epigenome-Transcriptome Association Analysis

An eQTL analysis between methylation sites and gene expressions was performed on the 34 DCM patients and 25 controls with high quality epigenome and transcriptome data from the same biopsy samples. MatrixEQTL (Shabalin A A. Matrix eQTL: ultra fast eQTL analysis via large matrix operations. Bioinformatics. 2012; 28:1353-8) and linear models were used to correlate the expression profiles of 19,418 genes with the 311,222 methylation sites in a range of 10,000 bp up- and downstream of the genes as well as in the gene body region. Epigenome-transcriptome associations were subsequently directionally verified in the cardiac tissue verification cohort.

To identify an epigenetic signature for DCM we filtered for methylation loci, which were associated with the disease and gene expression in myocardial discovery and verification cohort at an uncorrected significance level of p≤0.05. Conserved methylation differences in DCM across myocardial tissue and peripheral blood were identified by filtering for methylation loci that additionally showed conservation across tissues (kendall rank test for direct correlation p≤0.05) and deregulated methylation status in identical directions (directional p≤0.05). To minimize the effect of blood cell heterogeneity, we excluded all sites which have been shown to be associated with blood cell heterogeneity at a (Holm S. A simple sequentially rejective multiple test procedure. Scandinavian Journal of Statistics. 1979; 6, 65-70) corrected F-statistics significance level p≤0.05 by Jaffe et al. (Jaffe A E and Irizarry R A. Accounting for cellular heterogeneity is critical in epigenome-wide association studies. Genome Biol. 2014; 15:R31). Finally, predictive DCM models were built for myocardial tissue and peripheral blood separately using the glm function of the R stats package based on logistic regression models and 5-fold cross-validation with 10 repeats in the discovery cohort and subsequently tested in the verification cohorts.

For aggregated analyses on gene or multi-gene level, aggregate significance level was then obtained using the simes procedure for all methylation loci (RØDLAND E A. Simes' procedure is ‘valid on average’. Biometrika. 93:742-746).

### Overrepresentation and Gene Ontology Analyses

Overrepresentation analyses for deregulated methylation sites in chromosome bands, discovery and verification cohorts as well as for methylation sites associated with disease state and gene expression was based on the fisher exact test on 2×2 contingency tables using a threshold of p≤0.05.

Identification of overrepresented GO terms was performed using the gometh function of the missMethyl package (Phipson B, Maksimovic J and Oshlack A. missMethyl: an R package for analyzing data from Illumina's HumanMethylation450 platform. Bioinformatics. 2016; 32:286-8), taking into account the probability of differential methylation based on the number of probes on the 450 k array per gene. This is particularly important, since severe bias when performing gene set analysis for genome-wide methylation data due to the differing numbers of methylation sites profiled for each gene has been reported (Geeleher P, Hartnett L, Egan L J, Golden A, Raja Ali R A and Seoighe C. Gene-set analysis is severely biased when applied to genome-wide methylation data. Bioinformatics. 2013; 29:1851-7). The applied approach models and compensates the effect of selection bias using the methodological framework originally developed by Young et al. (Young M D, Wakefield M J, Smyth G K and Oshlack A. Gene ontology analysis for RNA-seq: accounting for selection bias. Genome Biol. 2010; 11:R14).

Further data regarding the analysis carried out in Example 2 and results obtained therein are found in the following Tables 23 to 34.

TABLE 23
Binding-site Overrepresentation in DMR (Tissue Screening).

| Motif | P-Value | FDR |
|---|---|---|
| Smad2 | 0.00010351269 | 0.01193747 |
| BMAL1 | 0.00014737619 | 0.01193747 |
| Smad4 | 0.00076668415 | 0.04657606 |
| Olig2 | 0.00006106049 | 0.01193747 |

TABLE 24
Overrepresented Gene Ontology Terms of Replicated
DCM-associated and geneexpression associated
DMR (Tissue Screening + Replication).

| GO Biological Process | P-Value | FDR |
|---|---|---|
| biological adhesion | 2.768E−11 | 3.6969E−07 |
| homophilic cell adhesion via plasma | 9.9239E−11 | 6.6272E−07 |
| membrane adhesion molecules | | |
| cell adhesion | 1.5502E−10 | 6.9015E−07 |
| cell-cell adhesion via plasma-membrane | 3.477E−10 | 9.7543E−07 |
| adhesion molecules | | |
| cell-cell adhesion | 3.6517E−10 | 9.7543E−07 |
| cardiac muscle cell differentiation | 3.6617E−06 | 0.00815094 |
| anatomical structure morphogenesis | 4.3621E−06 | 0.00832288 |
| muscle contraction | 5.9186E−06 | 0.00988107 |
| cardiovascular system development | 8.7094E−06 | 0.01163223 |
| circulatory system development | 8.7094E−06 | 0.01163223 |
| muscle system process | 9.7189E−06 | 0.0118005 |
| cardiac muscle tissue development | 1.5042E−05 | 0.01483882 |
| muscle filament sliding | 1.5554E−05 | 0.01483882 |
| actin-myosin filament sliding | 1.5554E−05 | 0.01483882 |
| multicellular organismal development | 1.8194E−05 | 0.01620023 |
| cardiac muscle cell development | 3.4344E−05 | 0.02675178 |
| myosin filament organization | 3.6108E−05 | 0.02675178 |
| cardiocyte differentiation | 3.7612E−05 | 0.02675178 |
| tissue development | 3.8057E−05 | 0.02675178 |
| cardiac cell development | 5.6864E−05 | 0.03725419 |
| skeletal muscle myosin thick filament | 6.1365E−05 | 0.03725419 |
| assembly | | |
| striated muscle myosin thick filament | 6.1365E−05 | 0.03725419 |
| assembly | | |

TABLE 25
Baseline Characteristics of Included Patients (Screening
stage, cardiac tissue & blood, n = 41)
Clinical characteristics

| Age, mean ± SD, y | 54.1 ± 12.3 | |
|---|---|---|
| Age at onset ± SD, y | 53.2 ± 12.6 | |
| Males, n. (%) | 31 | (75.6%) |
| BMI, mean ± SD, kg/m² | 27.1 ± 5.7 | |
| Atrial fibrillation, n. (%) | 6 | (14.6%) |
| Functional Class: | | |
| NYHA I, n. (%) | 6 | (14.6%) |
| NYHA II, n. (%) | 20 | (47.8%) |
| NYHA III, n. (%) | 14 | (34%) |
| NYHA IV, n. (%) | 1 | (2.4%) |
| Family history of SCD or DCM, n. (%) | 9 | (21.9%) |

Clinical Biomarkers

| | |
|---|---|
| White blood cell count, mean ± SD,/nl | 7.7 ± 2.3 |
| Haemoglobin, mean ± SD, g/dl | 14.3 ± 1.5 |
| eGFR, mean ± SD, mL/min/1.73 m² | 87.4 ± 17.8 |
| Creatinine ± SD, mg/dl | 0.9 ± 0.2 |
| NT-proBNP, median (1Q | 3Q), ng/l |
| 812 (109 | 2255) |
| hs-TNT, median (1Q | 3Q), pg/ml |
| 12 (8 | 36) |

Medications

| | | |
|---|---|---|
| ß-Blocker | 38 | (92.7%) |
| ACE inhibitor or ARB | 40 | (97.6%) |
| Loop diuretic | 18 | (43.9%) |
| Aldosterone antagonist | 20 | (48.9%) |

MRI

| | |
|---|---|
| LV ejection fraction, mean ± SD, % | 37 ± 15 |
| LV-EDV index, mean ± SD, mL/m² | 126.1 ± 44.3 |
| LV-EDD mm ± SD, mm | 61.2 ± 9.8 |
| RV-EDD mean ± SD, mm 48.0 ± 7.8 | |

ACE, angiotensin-converting enzyme; ARB, angiotensin II receptor blocker; DCM, dilated cardiomyopathy; EDD: end-diastolic diameter; EDV: end-diastolic volume; GFR: Glomerular filtration rate; LV: left ventricular; n: number; NYHA, New York Heart Association; SCD: sudden cardiac death; SD: standard deviation; 1Q: first quartile; 3Q: third Quartile.

TABLE 26
Baseline Characteristics of Included HTX Controls
(Screening stage, cardiac tissue, n = 31)
Basic characteristics

| Age, mean ± SD, y | 54.1 ± 11.7 | |
|---|---|---|
| Males, n. (%) | 24 | (77.4%) |

| | |
|---|---|
| BMI, mean ± SD, kg/m² | 24.4 ± 4 |
| Atrial fibrillation, n. | (%) 0 (0%) |

Laboratory tests

| | |
|---|---|
| White blood cell count, mean ± SD,/nl | 6.6 ± 2.9 |
| Haemoglobin, mean ± SD, g/dl | 12.7 ± 2.1 |
| Creatinine ± SD, mg/dl | 1.3 ± 0.4 |

Medications

| | | |
|---|---|---|
| Aspirin | 14 | (45.2%) |
| ß-Blocker | 19 | (61.3%) |
| ACE inhibitor or ARB | 25 | (80.1%) |
| Diuretic | 14 | (45.2%) |
| Steroid | 9 | (29%) |
| Tacrolimus | 21 | (74%) |
| Mycophenolat | 27 | (87.1%) |
| Everolimus | 5 | (16.1%) |
| Ciclosporin | 5 | (16.1%) |
| Sirolimus | 1 | (3.2%) |

Echocardiography

| | |
|---|---|
| LV ejection fraction, mean ± SD, % | 60.6 ± 3.1 |

ACE, angiotensin-converting enzyme;
ARB, angiotensin II receptor blocker;
EMB: endomyocardial biopsy;
n: number;
SD: standard deviation

TABLE 27
Baseline Characteristics of Included Clinical Controls
(Screening stage, blood methylation, n = 31)
Basic characteristics

| Age, mean ± SD, y | 65.7 ± .11 | |
|---|---|---|
| Males, n. (%) | 19 | (61.3%) |
| BMI, mean ± SD, kg/m² | 27.9 ± 4.2 | |
| Atrial fibrillation, n. (%) | 3 | (9.7%) |

Laboratory tests

| | |
|---|---|
| White blood cell count, mean ± SD,/nl | 7.7 ± 2.7 |
| Haemoglobin, mean ± SD, g/dl | 14.4 ± 1.1 |
| Creatinine ± SD, mg/dl | 0.8 ± 0.2 |

Medications

| | | |
|---|---|---|
| Aspirin | 22 | (71.0%) |
| ß-Blocker | 19 | (61.3%) |
| ACE inhibitor or ARB | 14 | (45.2%) |
| Diuretic | 9 | (29.0%) |
| Statin | 13 | (41.9%) |

Echocardiography

| | |
|---|---|
| LV ejection fraction, mean ± SD, % | 61.5 ± 3.4 |

ACE, angiotensin-converting enzyme; ARB, angiotensin II receptor blocker; n: number; SD: standard deviation.

TABLE 28
Baseline Characteristics of Included DCM Patients
(Replication stage, cardiac tissue, n = 18)
Basic characteristics

| Age, mean ± SD, y | 58.2 ± 8.8 | |
|---|---|---|
| Age at onset ± SD, y | 52.0 ± 11.5 | |
| Males, n. (%) | 13 | (72.2%) |
| Atrial fibrillation, n. (%) | 10 | (55.5%) |
| Functional classes: | | |
| NYHA I, n. (%) | 1 | (5.6%) |
| NYHA II, n. (%) | 4 | (22.2%) |
| NYHA III, n. (%) | 10 | (55.6%) |
| NYHA IV, n. (%) | 1 | (16.7%) |

Clinical biomarkers

| White blood cell count, mean ± SD,/nl | 8.4 ± 3.4 | |
|---|---|---|
| Haemoglobin, mean ± SD, g/dl | 13.3 ± 1.9 | |
| Creatinine ± SD, mg/dl | 1.5 ± 0.8 | |
| NT-proBNP, median (1Q, 3Q), ng/l | 5641 | (2201; 10309) |

Medications

| | | |
|---|---|---|
| ß-Blocker | 15 | (83.3%) |
| ACE inhibitor or ARB | 17 | (94.4%) |
| Diuretic | 17 | (94.4%) |

Echocardiography

| | |
|---|---|
| LV ejection fraction, mean ± SD, % | 23 ± 8 |
| LV-EDD, mean ± SD, mm/m² | 61 ± 8 |

ACE, angiotensin-converting enzyme; ARB, angiotensin II receptor blocker; DCM, dilated cardiomyopathy; EDD: end-diastolic diameter; LV: left ventricular; n: number; NYHA, New York Heart Association; SD: standard deviation; 1Q: first quartile; 3Q: third Quartile.

TABLE 29
Baseline Characteristics of Included Accident
Controls (Replication stage, cardiac tissue, n=8)
Basic characteristic

| | |
|---|---|
| Males, n. (%) | 7 (87.5%) |

n: number

TABLE 30
Baseline Characteristics of Included DCM patients
(Replication stage I, blood, n = 9)
Basic characteristics

| | |
|---|---|
| Age, mean ± SD, y | 53 ± 14.8 |
| Age at onset ± SD, y | 52.8 ± 15.1 |
| Males, n. (%) | 8 (88.8%) |
| Atrial fibrillation, n. (%) | 6 (66.7%) |
| Functional classes: | |
| NYHA I, n. (%) | 1 (22.2%) |
| NYHA II, n. (%) | 1 (22.2%) |
| NYHA III, n. (%) | 5 (55.6%) |
| NYHA IV, n. (%) | 0 (0%) |

Clinical biomarkers

| | |
|---|---|
| White blood cell count, mean ± SD,/nl | 8.4 ± 3.4 |
| Haemoglobin, mean ± SD, g/dl | 14.6 ± 1.5 |
| Creatinine ± SD, mg/dl | 1.0 ± 0.2 |
| NT-proBNP, median (1Q; 3Q), ng/l | 233 (144; 636) |

Medications, n. (%)

| | |
|---|---|
| ß-Blocker | 8 (88.8%) |
| ACE inhibitor or ARB | 9 (100%) |
| Diuretic | 6 (66.7%) |

Echocardiography

| | |
|---|---|
| LV ejection fraction, mean ± SD, % | 32 ± 12 |
| LV-EDD, mean ± SD, mm/m² | 57 ± 6 |

ACE, angiotensin-converting enzyme; ARB, angiotensin II receptor blocker; DCM, dilated cardiomyopathy; EDD: end-diastolic diameter; LV: left ventricular; n: number; NYHA, New York Heart Association; SD: standard deviation; 1Q: first quartile; 3Q: third Quartile.

TABLE 31
Baseline Characteristics of Included Controls
(Replication stage I, blood, n = 28)
Basic characteristics

| | |
|---|---|
| Age, mean ± SD, y | 59.6 ± .8.5 |
| Males, n. (%) | 22 (78.6%) |

TABLE 32
Baseline Characteristics of Included DCM patients
(Replication stage II, blood, n = 82)
Clinical characteristics

| | |
|---|---|
| Age, mean ± SD, y | 53.0 ± 13.4 |
| Males, n. (%) | 64 (78.0%) |
| BMI, mean ± SD, kg/m² | 28.3 ± 6.5 |
| Atrial fibrillation, n. (%) | 21 (25.6%) |

Clinical Biomarkers

| | |
|---|---|
| White blood cell count, mean ± SD,/nl | 7.6 ± 2.1 |
| Haemoglobin, mean ± SD, g/dl | 14.4 ± 1.6 |
| Creatinine ± SD, mg/dl | 1.3 ± 1.5 |
| NT-proBNP, median (1Q; 3Q), ng/l | 785 (144; 2626) |
| hs-TNT, median (1Q; 3Q), pg/ml | 12 (6; 23) |
| CRP, mean ± SD; mg/l | 8.3 (24.6) |

Echocradiography

| | |
|---|---|
| LV ejection fraction, mean ± SD, % | 30 ± 13 |
| LV-EDD mean ± SD, mm | 61.6 ± 10.2 |

EDD: end-diastolic diameter; LV: left ventricular; n: number; SD: standard deviation; IQ: first quartile; 3Q: third Quartile.

TABLE 33
Baseline Characteristics of Included Controls
(Replication stage II, blood, n = 109)
Clinical characteristics

| | |
|---|---|
| Age, mean ± SD, y | 62.2 ± 9.1 |
| Males, n. (%) | 81 (74.3%) |
| BMI, mean ± SD, kg/m² | 24.9 ± 2.7 |
| Atrial fibrillation, n. (%) | 0 (0%) |

Clinical Biomarkers

| | |
|---|---|
| White blood cell count, mean ± SD,/nl | 7.6 ± 1.3 |
| Haemoglobin, mean ± SD, g/dl | 14.6 ± 1.0 |
| Creatinine ± SD, mg/dl | 0.85 ± 0.1 |
| CRP, mean ± SD; mg/l | 5.1 (2.6) |

Echocradiography

| | |
|---|---|
| LV ejection fraction, mean ± SD, % | 59.1 ± 8.7 |
| LV-EDD mean ± SD, mm | 46.8 ± 4.5 |

EDD: end-diastolic diameter;
LV: left ventricular;
n: number;
SD: standard deviation

TABLE 34
Loci associated with DCM and RNA expression.

| | | | Pearson | |
|---|---|---|---|---|
| | | | Correlation | |
| | | p-Value DCM | Methylation- | p-Value |
| CpG Site | Nearby Gene | Association | RNA | Correlation |
| cg14523204 | ENSG00000138835 | 4.74E−06 | −0.3708 | 3.84E−03 |
| cg16254946 | ENSG00000174332 | 7.42E−06 | −0.4643 | 2.12E−04 |
| cg21518947 | ENSG00000269913 | 1.48E−05 | −0.3814 | 2.88E−03 |
| ch.3.1226245F | ENSG00000187672 | 1.50E−05 | −0.3289 | 1.10E−02 |
| cg21363050 | ENSG00000134853 | 1.54E−05 | −0.3526 | 6.16E−03 |
| cg21363050 | ENSG00000145216 | 1.54E−05 | −0.3208 | 1.32E−02 |
| cg25924602 | ENSG00000163145 | 2.92E−05 | −0.4888 | 8.58E−05 |
| cg11822932 | ENSG00000135363 | 3.05E−05 | −0.3422 | 7.98E−03 |
| ch.10.2770541R | ENSG00000150760 | 4.32E−05 | −0.2870 | 2.75E−02 |
| cg03001305 | ENSG00000126561 | 7.10E−05 | −0.4174 | 1.00E−03 |
| cg11970163 | ENSG00000135842 | 7.68E−05 | −0.6979 | 8.10E−10 |
| cg02801277 | ENSG00000101638 | 1.08E−04 | −0.6542 | 1.92E−08 |
| cg02801277 | ENSG00000270112 | 1.08E−04 | −0.6609 | 1.23E−08 |
| cg03600605 | ENSG00000170421 | 1.16E−04 | −0.7376 | 2.67E−11 |
| cg08732466 | ENSG00000177133 | 1.17E−04 | −0.3334 | 9.86E−03 |
| cg08732466 | ENSG00000142611 | 1.17E−04 | −0.3271 | 1.15E−02 |
| cg13909178 | ENSG00000151702 | 1.24E−04 | 0.3906 | 2.22E−03 |
| cg02215357 | ENSG00000139675 | 1.34E−04 | −0.3780 | 3.16E−03 |
| cg06783197 | ENSG00000179364 | 1.36E−04 | 0.2631 | 4.41E−02 |
| cg19223064 | ENSG00000165757 | 1.37E−04 | −0.2907 | 2.55E−02 |
| cg21144009 | ENSG00000076356 | 1.48E−04 | −0.4817 | 1.12E−04 |
| cg08840665 | ENSG00000183011 | 1.53E−04 | −0.2968 | 2.24E−02 |
| cg08840665 | ENSG00000167874 | 1.53E−04 | −0.2636 | 4.37E−02 |
| cg08840665 | ENSG00000182224 | 1.53E−04 | −0.2823 | 3.03E−02 |
| cg05990080 | ENSG00000144677 | 2.00E−04 | −0.6816 | 2.80E−09 |
| cg23664174 | ENSG00000072201 | 2.11E−04 | −0.4015 | 1.62E−03 |
| cg19514721 | ENSG00000231185 | 2.25E−04 | −0.2675 | 4.05E−02 |
| cg16045271 | ENSG00000076356 | 2.26E−04 | −0.5452 | 8.00E−06 |
| cg11702448 | ENSG00000105401 | 2.27E−04 | −0.3360 | 9.27E−03 |
| ch.7.1171004F | ENSG00000106070 | 2.33E−04 | −0.3738 | 3.54E−03 |
| cg09777256 | ENSG00000155657 | 3.15E−04 | −0.3471 | 7.07E−03 |
| cg17326555 | ENSG00000092607 | 3.22E−04 | 0.6010 | 4.84E−07 |
| cg09990481 | ENSG00000107796 | 3.94E−04 | −0.3666 | 4.29E−03 |
| cg09990481 | ENSG00000138134 | 3.94E−04 | −0.4206 | 9.11E−04 |
| cg04430582 | ENSG00000267532 | 3.98E−04 | −0.2852 | 2.86E−02 |
| cg04430582 | ENSG00000219200 | 3.98E−04 | 0.2680 | 4.02E−02 |
| cg19677302 | ENSG00000057294 | 4.11E−04 | −0.2904 | 2.57E−02 |
| cg14524975 | ENSG00000139626 | 4.46E−04 | −0.3850 | 2.61E−03 |
| cg20950633 | ENSG00000206561 | 4.50E−04 | −0.3283 | 1.11E−02 |
| cg09779027 | ENSG00000171105 | 4.90E−04 | −0.3363 | 9.20E−03 |
| cg19201144 | ENSG00000186684 | 5.15E−04 | −0.2971 | 2.23E−02 |
| cg23436746 | ENSG00000188730 | 5.37E−04 | −0.5435 | 8.67E−06 |
| cg26512226 | ENSG00000175084 | 5.44E−04 | −0.3785 | 3.12E−03 |
| cg14174232 | ENSG00000178031 | 5.46E−04 | −0.5017 | 5.16E−05 |
| cg00767058 | ENSG00000150401 | 5.49E−04 | −0.6773 | 3.85E−09 |
| cg00767058 | ENSG00000153531 | 5.49E−04 | −0.5235 | 2.09E−05 |
| cg00857536 | ENSG00000165633 | 5.70E−04 | −0.3722 | 3.70E−03 |
| cg06357561 | ENSG00000126561 | 5.71E−04 | −0.3614 | 4.92E−03 |
| cg14039237 | ENSG00000148339 | 6.48E−04 | −0.2674 | 4.06E−02 |
| cg01876531 | ENSG00000076356 | 6.81E−04 | −0.5424 | 9.10E−06 |
| cg03721976 | ENSG00000266040 | 6.83E−04 | 0.2792 | 3.23E−02 |
| cg03721976 | ENSG00000108292 | 6.83E−04 | 0.3891 | 2.32E−03 |
| cg05819249 | ENSG00000113504 | 7.00E−04 | −0.5639 | 3.31E−06 |
| cg07249742 | ENSG00000082781 | 7.21E−04 | −0.3176 | 1.42E−02 |
| cg07654843 | ENSG00000133454 | 7.44E−04 | −0.2883 | 2.68E−02 |
| cg07164133 | ENSG00000114541 | 7.57E−04 | −0.3178 | 1.42E−02 |
| cg21829328 | ENSG00000099958 | 7.71E−04 | 0.3002 | 2.09E−02 |
| cg23882945 | ENSG00000073331 | 7.76E−04 | 0.3002 | 2.09E−02 |
| cg08569786 | ENSG00000119771 | 7.99E−04 | 0.3798 | 3.01E−03 |
| cg09537551 | ENSG00000104375 | 8.03E−04 | 0.3136 | 1.56E−02 |
| cg10587082 | ENSG00000076356 | 8.05E−04 | −0.4303 | 6.69E−04 |
| cg12563372 | ENSG00000221818 | 8.70E−04 | 0.4505 | 3.43E−04 |
| cg17486234 | ENSG00000104332 | 8.85E−04 | −0.3435 | 7.74E−03 |
| cg11235297 | ENSG00000113504 | 9.30E−04 | −0.4271 | 7.41E−04 |
| cg24128630 | ENSG00000182224 | 9.33E−04 | −0.4742 | 1.48E−04 |
| cg24128630 | ENSG00000167874 | 9.33E−04 | −0.4953 | 6.65E−05 |
| cg24128630 | ENSG00000132510 | 9.33E−04 | −0.2906 | 2.56E−02 |
| cg24128630 | ENSG00000183011 | 9.33E−04 | −0.2985 | 2.17E−02 |
| cg08140459 | ENSG00000119681 | 9.58E−04 | −0.5521 | 5.83E−06 |
| cg14624207 | ENSG00000162337 | 9.89E−04 | −0.3000 | 2.10E−02 |
| cg15227911 | ENSG00000183011 | 1.01E−03 | −0.2836 | 2.95E−02 |
| cg10402018 | ENSG00000181754 | 1.05E−03 | 0.3441 | 7.61E−03 |
| cg12140144 | ENSG00000177133 | 1.08E−03 | −0.3694 | 3.99E−03 |
| cg12140144 | ENSG00000142611 | 1.08E−03 | −0.3294 | 1.09E−02 |
| cg04201373 | ENSG00000030110 | 1.11E−03 | 0.3854 | 2.58E−03 |
| cg05678871 | ENSG00000174780 | 1.18E−03 | −0.4142 | 1.11E−03 |
| cg11909137 | ENSG00000101665 | 1.18E−03 | −0.3223 | 1.28E−02 |
| cg12475142 | ENSG00000226137 | 1.21E−03 | −0.4950 | 6.72E−05 |
| cg12475142 | ENSG00000175866 | 1.21E−03 | −0.3227 | 1.27E−02 |
| cg26498574 | ENSG00000122367 | 1.25E−03 | −0.5197 | 2.47E−05 |
| cg14741228 | ENSG00000139146 | 1.25E−03 | −0.6173 | 1.91E−07 |
| cg04101806 | ENSG00000230393 | 1.27E−03 | −0.2700 | 3.86E−02 |
| cg20462242 | ENSG00000142611 | 1.30E−03 | −0.3449 | 7.46E−03 |
| cg02711479 | ENSG00000181817 | 1.36E−03 | −0.2693 | 3.91E−02 |
| cg17810966 | ENSG00000163110 | 1.41E−03 | −0.3464 | 7.20E−03 |
| cg00434119 | ENSG00000058866 | 1.41E−03 | −0.4576 | 2.69E−04 |
| cg24678869 | ENSG00000198837 | 1.42E−03 | 0.3573 | 5.46E−03 |
| cg15647725 | ENSG00000113504 | 1.47E−03 | −0.4628 | 2.23E−04 |
| cg04864441 | ENSG00000155093 | 1.52E−03 | −0.2869 | 2.76E−02 |
| cg22219450 | ENSG00000166016 | 1.53E−03 | −0.4770 | 1.34E−04 |
| cg14703829 | ENSG00000244486 | 1.54E−03 | 0.4641 | 2.14E−04 |
| cg14703829 | ENSG00000099910 | 1.54E−03 | 0.3490 | 6.74E−03 |
| cg05905699 | ENSG00000155657 | 1.55E−03 | −0.3315 | 1.03E−02 |
| cg01179095 | ENSG00000175206 | 1.61E−03 | −0.3960 | 1.90E−03 |
| cg01179095 | ENSG00000242349 | 1.61E−03 | −0.2912 | 2.52E−02 |
| cg03221266 | ENSG00000107796 | 1.62E−03 | −0.4799 | 1.20E−04 |
| cg03221266 | ENSG00000138134 | 1.62E−03 | −0.4423 | 4.53E−04 |
| cg20979153 | ENSG00000171940 | 1.66E−03 | −0.4400 | 4.88E−04 |
| cg20979153 | ENSG00000197670 | 1.66E−03 | −0.3672 | 4.23E−03 |
| cg09550083 | ENSG00000143995 | 1.75E−03 | −0.2755 | 3.47E−02 |
| cg09284275 | ENSG00000133392 | 1.85E−03 | −0.5835 | 1.24E−06 |
| cg04685570 | ENSG00000255399 | 1.86E−03 | 0.4147 | 1.09E−03 |
| cg04685570 | ENSG00000089225 | 1.86E−03 | 0.2855 | 2.84E−02 |
| cg14711976 | ENSG00000186204 | 1.88E−03 | 0.3017 | 2.02E−02 |
| cg16201146 | ENSG00000185052 | 1.89E−03 | −0.5150 | 3.01E−05 |
| cg04109883 | ENSG00000165633 | 1.89E−03 | −0.4309 | 6.57E−04 |
| cg13364311 | ENSG00000116106 | 1.91E−03 | −0.5640 | 3.29E−06 |
| cg03850035 | ENSG00000116106 | 1.92E−03 | −0.5553 | 4.99E−06 |
| cg12509665 | ENSG00000075240 | 2.00E−03 | −0.3653 | 4.44E−03 |
| cg12509665 | ENSG00000100422 | 2.00E−03 | −0.3899 | 2.27E−03 |
| cg03256938 | ENSG00000177133 | 2.04E−03 | −0.3870 | 2.46E−03 |
| cg03256938 | ENSG00000142611 | 2.04E−03 | −0.4447 | 4.17E−04 |
| cg08127462 | ENSG00000197956 | 2.05E−03 | −0.2956 | 2.30E−02 |
| cg08127462 | ENSG00000196154 | 2.05E−03 | −0.4038 | 1.52E−03 |
| cg08127462 | ENSG00000188015 | 2.05E−03 | −0.3163 | 1.47E−02 |
| cg14138002 | ENSG00000101665 | 2.10E−03 | −0.3623 | 4.80E−03 |
| cg22045225 | ENSG00000255399 | 2.12E−03 | 0.3315 | 1.03E−02 |
| cg13379195 | ENSG00000108405 | 2.12E−03 | −0.4515 | 3.31E−04 |
| cg27010834 | ENSG00000120057 | 2.12E−03 | −0.2852 | 2.86E−02 |
| cg17250863 | ENSG00000131069 | 2.16E−03 | −0.5154 | 2.95E−05 |
| cg03541338 | ENSG00000148908 | 2.18E−03 | −0.2633 | 4.39E−02 |
| cg16254190 | ENSG00000227959 | 2.19E−03 | −0.3747 | 3.46E−03 |
| cg25608061 | ENSG00000128652 | 2.26E−03 | 0.3332 | 9.91E−03 |
| cg08029603 | ENSG00000223764 | 2.31E−03 | −0.3487 | 6.80E−03 |
| cg08029603 | ENSG00000187634 | 2.31E−03 | −0.4263 | 7.60E−04 |
| cg10586672 | ENSG00000131389 | 2.47E−03 | −0.3180 | 1.41E−02 |
| cg26585100 | ENSG00000166558 | 2.52E−03 | 0.2661 | 4.17E−02 |
| cg26585100 | ENSG00000140943 | 2.52E−03 | 0.3342 | 9.68E−03 |
| cg14340889 | ENSG00000119681 | 2.52E−03 | −0.3656 | 4.41E−03 |
| cg00727912 | ENSG00000101193 | 2.53E−03 | 0.3073 | 1.79E−02 |
| cg02551743 | ENSG00000143995 | 2.55E−03 | −0.2565 | 4.99E−02 |
| cg27396830 | ENSG00000162490 | 2.58E−03 | 0.3545 | 5.87E−03 |
| cg04025127 | ENSG00000142949 | 2.61E−03 | −0.3045 | 1.90E−02 |
| cg03502979 | ENSG00000150401 | 2.63E−03 | −0.5884 | 9.56E−07 |
| cg03502979 | ENSG00000153531 | 2.63E−03 | −0.4847 | 1.00E−04 |
| cg21647227 | ENSG00000092607 | 2.78E−03 | 0.5428 | 8.92E−06 |
| cg27627006 | ENSG00000184384 | 2.88E−03 | −0.5361 | 1.21E−05 |
| cg13510418 | ENSG00000070159 | 2.91E−03 | −0.6498 | 2.58E−08 |
| cg26112170 | ENSG00000150401 | 2.97E−03 | −0.6407 | 4.61E−08 |
| cg26112170 | ENSG00000153531 | 2.97E−03 | −0.5585 | 4.29E−06 |
| cg15513743 | ENSG00000092607 | 2.98E−03 | 0.2882 | 2.69E−02 |
| cg05377733 | ENSG00000137809 | 3.00E−03 | −0.5036 | 4.79E−05 |
| cg22627753 | ENSG00000217801 | 3.03E−03 | −0.4864 | 9.37E−05 |
| cg10308749 | ENSG00000135547 | 3.09E−03 | −0.3853 | 2.58E−03 |
| cg10308749 | ENSG00000237742 | 3.09E−03 | −0.3554 | 5.74E−03 |
| cg23546474 | ENSG00000135903 | 3.18E−03 | 0.3283 | 1.11E−02 |
| cg14310606 | ENSG00000244187 | 3.36E−03 | 0.4424 | 4.51E−04 |
| cg14310606 | ENSG00000273066 | 3.36E−03 | 0.4970 | 6.22E−05 |
| cg14310606 | ENSG00000196642 | 3.36E−03 | 0.3858 | 2.55E−03 |
| cg14153927 | ENSG00000124440 | 3.45E−03 | 0.3457 | 7.32E−03 |
| cg14153927 | ENSG00000011485 | 3.45E−03 | 0.5123 | 3.36E−05 |
| cg03715070 | ENSG00000082641 | 3.46E−03 | 0.5023 | 5.05E−05 |
| cg14851471 | ENSG00000250230 | 3.47E−03 | 0.2703 | 3.84E−02 |
| cg14851471 | ENSG00000011347 | 3.47E−03 | 0.5171 | 2.75E−05 |
| cg08310088 | ENSG00000169181 | 3.53E−03 | −0.4849 | 9.93E−05 |
| cg07202214 | ENSG00000236304 | 3.58E−03 | −0.3374 | 8.97E−03 |
| cg05658236 | ENSG00000186564 | 3.65E−03 | 0.2767 | 3.39E−02 |
| cg00668685 | ENSG00000181852 | 3.66E−03 | 0.3568 | 5.54E−03 |
| cg22588546 | ENSG00000133026 | 3.68E−03 | −0.6174 | 1.91E−07 |
| cg13720639 | ENSG00000197555 | 3.73E−03 | −0.2928 | 2.44E−02 |
| cg19170009 | ENSG00000026025 | 3.83E−03 | −0.3703 | 3.89E−03 |
| cg09486407 | ENSG00000167522 | 3.86E−03 | −0.2851 | 2.86E−02 |
| cg09608533 | ENSG00000121898 | 3.92E−03 | −0.4101 | 1.26E−03 |
| cg24796554 | ENSG00000151702 | 3.94E−03 | −0.5377 | 1.13E−05 |
| cg23248351 | ENSG00000154188 | 4.00E−03 | −0.3369 | 9.08E−03 |
| cg20669834 | ENSG00000065534 | 4.17E−03 | −0.5337 | 1.34E−05 |
| cg20669834 | ENSG00000239523 | 4.17E−03 | −0.3517 | 6.31E−03 |
| cg18444673 | ENSG00000126264 | 4.31E−03 | −0.3903 | 2.24E−03 |
| cg18444673 | ENSG00000167604 | 4.31E−03 | −0.3955 | 1.93E−03 |
| cg18444673 | ENSG00000011600 | 4.31E−03 | −0.3638 | 4.62E−03 |
| cg20330521 | ENSG00000187955 | 4.33E−03 | −0.5127 | 3.30E−05 |
| cg00642359 | ENSG00000255399 | 4.37E−03 | 0.4152 | 1.08E−03 |
| cg00642359 | ENSG00000089225 | 4.37E−03 | 0.2590 | 4.76E−02 |
| cg20054157 | ENSG00000225383 | 4.37E−03 | −0.2633 | 4.39E−02 |
| cg16529477 | ENSG00000135903 | 4.40E−03 | 0.5013 | 5.25E−05 |
| cg22871653 | ENSG00000092607 | 4.42E−03 | 0.5353 | 1.25E−05 |
| cg14529268 | ENSG00000186510 | 4.42E−03 | −0.3538 | 5.98E−03 |
| cg14529268 | ENSG00000183888 | 4.42E−03 | −0.3591 | 5.22E−03 |
| cg16022049 | ENSG00000134531 | 4.49E−03 | −0.5527 | 5.66E−06 |
| cg21660452 | ENSG00000110076 | 4.50E−03 | 0.2638 | 4.35E−02 |
| cg00500213 | ENSG00000272829 | 4.53E−03 | 0.3226 | 1.27E−02 |
| cg11382082 | ENSG00000177738 | 4.53E−03 | −0.2607 | 4.61E−02 |
| cg13633756 | ENSG00000161558 | 4.57E−03 | 0.3219 | 1.29E−02 |
| cg22070156 | ENSG00000198542 | 4.60E−03 | −0.3882 | 2.38E−03 |
| cg03927133 | ENSG00000137825 | 4.60E−03 | 0.2962 | 2.28E−02 |
| cg21015470 | ENSG00000183715 | 4.63E−03 | −0.3624 | 4.79E−03 |
| cg01673674 | ENSG00000156466 | 4.68E−03 | 0.3843 | 2.66E−03 |
| cg00319334 | ENSG00000238184 | 4.78E−03 | −0.2793 | 3.22E−02 |
| cg00319334 | ENSG00000110651 | 4.78E−03 | −0.2582 | 4.84E−02 |
| cg09533305 | ENSG00000168135 | 4.97E−03 | −0.3759 | 3.34E−03 |
| cg11677852 | ENSG00000113504 | 4.98E−03 | −0.3864 | 2.50E−03 |
| cg15718932 | ENSG00000162645 | 5.14E−03 | −0.7077 | 3.68E−10 |
| cg16906137 | ENSG00000187720 | 5.20E−03 | −0.4699 | 1.73E−04 |
| cg09417209 | ENSG00000189067 | 5.22E−03 | −0.2857 | 2.83E−02 |
| cg27097542 | ENSG00000189067 | 5.22E−03 | −0.2818 | 3.06E−02 |
| cg21170682 | ENSG00000255090 | 5.28E−03 | −0.2711 | 3.78E−02 |
| cg12881854 | ENSG00000145216 | 5.46E−03 | 0.3088 | 1.73E−02 |
| cg07087686 | ENSG00000162367 | 5.47E−03 | 0.2603 | 4.65E−02 |
| cg19694465 | ENSG00000158286 | 5.47E−03 | −0.2988 | 2.15E−02 |
| cg10211776 | ENSG00000156675 | 5.90E−03 | −0.2737 | 3.60E−02 |
| cg14404746 | ENSG00000212864 | 5.92E−03 | −0.2784 | 3.28E−02 |
| cg18745416 | ENSG00000091831 | 6.00E−03 | −0.3405 | 8.32E−03 |
| cg16015205 | ENSG00000130695 | 6.01E−03 | 0.4868 | 9.25E−05 |
| cg19757176 | ENSG00000143549 | 6.03E−03 | −0.3861 | 2.52E−03 |
| cg06170425 | ENSG00000162104 | 6.11E−03 | −0.4396 | 4.95E−04 |
| cg00428638 | ENSG00000171105 | 6.17E−03 | −0.4034 | 1.53E−03 |
| cg13420075 | ENSG00000129009 | 6.23E−03 | −0.2758 | 3.45E−02 |
| cg06687489 | ENSG00000128833 | 6.23E−03 | −0.4759 | 1.39E−04 |
| cg08671647 | ENSG00000169851 | 6.24E−03 | −0.6658 | 8.74E−09 |
| cg04659582 | ENSG00000185404 | 6.27E−03 | −0.4246 | 8.03E−04 |
| cg04659582 | ENSG00000067066 | 6.27E−03 | −0.3920 | 2.14E−03 |
| cg04857136 | ENSG00000155465 | 6.35E−03 | −0.2565 | 4.99E−02 |
| cg11152884 | ENSG00000156427 | 6.39E−03 | −0.4724 | 1.58E−04 |
| cg24944328 | ENSG00000182580 | 6.42E−03 | −0.6562 | 1.68E−08 |
| cg24944328 | ENSG00000145191 | 6.42E−03 | 0.3526 | 6.16E−03 |
| cg23679756 | ENSG00000166250 | 6.50E−03 | 0.2848 | 2.88E−02 |
| cg12156944 | ENSG00000156466 | 6.55E−03 | 0.4229 | 8.46E−04 |
| cg14369455 | ENSG00000073331 | 6.83E−03 | 0.4025 | 1.58E−03 |
| cg26395694 | ENSG00000150995 | 6.88E−03 | −0.2903 | 2.57E−02 |
| cg07216529 | ENSG00000107796 | 7.06E−03 | −0.4804 | 1.18E−04 |
| cg07216529 | ENSG00000138134 | 7.06E−03 | −0.3827 | 2.78E−03 |
| cg17245125 | ENSG00000119771 | 7.07E−03 | −0.4431 | 4.41E−04 |
| cg03719978 | ENSG00000167874 | 7.19E−03 | −0.4875 | 8.98E−05 |
| cg03719978 | ENSG00000182224 | 7.19E−03 | −0.5057 | 4.39E−05 |
| cg03719978 | ENSG00000132510 | 7.19E−03 | −0.2698 | 3.88E−02 |
| cg03719978 | ENSG00000183011 | 7.19E−03 | −0.3222 | 1.28E−02 |
| cg01085362 | ENSG00000121297 | 7.25E−03 | 0.3323 | 1.01E−02 |
| cg00160583 | ENSG00000084636 | 7.26E−03 | −0.5230 | 2.14E−05 |
| cg24655428 | ENSG00000129009 | 7.26E−03 | −0.4278 | 7.24E−04 |
| cg10995873 | ENSG00000241560 | 7.31E−03 | 0.2885 | 2.67E−02 |
| cg00632560 | ENSG00000203883 | 7.32E−03 | −0.2762 | 3.42E−02 |
| cg21198219 | ENSG00000151702 | 7.43E−03 | 0.4289 | 7.00E−04 |
| cg10766942 | ENSG00000172954 | 7.43E−03 | −0.3409 | 8.24E−03 |
| cg13740187 | ENSG00000143549 | 7.50E−03 | −0.5243 | 2.03E−05 |
| cg10464312 | ENSG00000143995 | 7.77E−03 | −0.2765 | 3.40E−02 |
| cg04296837 | ENSG00000132780 | 7.82E−03 | −0.2743 | 3.55E−02 |
| cg04296837 | ENSG00000159592 | 7.82E−03 | −0.3054 | 1.87E−02 |
| cg22646937 | ENSG00000145936 | 7.86E−03 | −0.5758 | 1.83E−06 |
| cg16056219 | ENSG00000119681 | 8.04E−03 | −0.4164 | 1.04E−03 |
| cg27110491 | ENSG00000183696 | 8.19E−03 | −0.4189 | 9.58E−04 |
| cg16458436 | ENSG00000255399 | 8.23E−03 | 0.5092 | 3.82E−05 |
| cg16458436 | ENSG00000089225 | 8.23E−03 | 0.2634 | 4.39E−02 |
| cg14435109 | ENSG00000163082 | 8.33E−03 | −0.6121 | 2.58E−07 |
| cg04278110 | ENSG00000065413 | 8.35E−03 | −0.2791 | 3.23E−02 |
| cg16419054 | ENSG00000179364 | 8.52E−03 | −0.2830 | 2.99E−02 |
| cg04347708 | ENSG00000151702 | 8.62E−03 | 0.2923 | 2.47E−02 |
| cg09229492 | ENSG00000140807 | 8.73E−03 | −0.3386 | 8.70E−03 |
| cg16849268 | ENSG00000170873 | 8.80E−03 | −0.3142 | 1.54E−02 |
| cg26118208 | ENSG00000226900 | 8.85E−03 | −0.4180 | 9.87E−04 |
| ch.17.45797972F | ENSG00000108829 | 8.93E−03 | −0.3638 | 4.62E−03 |
| ch.17.45797972F | ENSG00000108826 | 8.93E−03 | 0.2675 | 4.05E−02 |
| ch.17.45797972F | ENSG00000015532 | 8.93E−03 | 0.3119 | 1.62E−02 |
| cg01056398 | ENSG00000016391 | 8.94E−03 | −0.2855 | 2.84E−02 |
| cg10525432 | ENSG00000112902 | 8.97E−03 | −0.3857 | 2.56E−03 |
| cg25401771 | ENSG00000151914 | 9.00E−03 | −0.3145 | 1.53E−02 |
| cg19095920 | ENSG00000164107 | 9.15E−03 | −0.3011 | 2.05E−02 |
| cg20889818 | ENSG00000259275 | 9.38E−03 | 0.2622 | 4.48E−02 |
| cg02769104 | ENSG00000253368 | 9.46E−03 | −0.2715 | 3.75E−02 |
| cg02769104 | ENSG00000158246 | 9.46E−03 | −0.2671 | 4.08E−02 |
| cg15246805 | ENSG00000112319 | 9.57E−03 | −0.5942 | 6.99E−07 |
| cg23207527 | ENSG00000112183 | 9.79E−03 | −0.4572 | 2.72E−04 |
| cg09013655 | ENSG00000198756 | 9.93E−03 | −0.5051 | 4.50E−05 |
| cg09535924 | ENSG00000143995 | 9.95E−03 | −0.2974 | 2.21E−02 |
| cg19910802 | ENSG00000227051 | 1.01E−02 | −0.6557 | 1.73E−08 |
| cg05576828 | ENSG00000120875 | 1.05E−02 | −0.2864 | 2.79E−02 |
| cg24755996 | ENSG00000118004 | 1.07E−02 | −0.3357 | 9.33E−03 |
| cg11201447 | ENSG00000249859 | 1.08E−02 | −0.7180 | 1.54E−10 |
| cg12719753 | ENSG00000129009 | 1.08E−02 | −0.3080 | 1.76E−02 |
| cg02075791 | ENSG00000165338 | 1.09E−02 | −0.3013 | 2.04E−02 |
| cg00609473 | ENSG00000197324 | 1.10E−02 | 0.2975 | 2.21E−02 |
| cg02386822 | ENSG00000163462 | 1.10E−02 | −0.3059 | 1.84E−02 |
| cg24242519 | ENSG00000197872 | 1.11E−02 | −0.2960 | 2.28E−02 |
| cg10065530 | ENSG00000125843 | 1.11E−02 | 0.2564 | 5.00E−02 |
| cg24164786 | ENSG00000119681 | 1.12E−02 | −0.2963 | 2.27E−02 |
| cg01640635 | ENSG00000100196 | 1.13E−02 | −0.3298 | 1.07E−02 |
| cg06903465 | ENSG00000177570 | 1.13E−02 | −0.3060 | 1.84E−02 |
| cg25079691 | ENSG00000221818 | 1.14E−02 | 0.3903 | 2.24E−03 |
| cg17871993 | ENSG00000169902 | 1.14E−02 | −0.2810 | 3.11E−02 |
| cg24720355 | ENSG00000092607 | 1.14E−02 | 0.5253 | 1.94E−05 |
| cg14402017 | ENSG00000142102 | 1.14E−02 | 0.2583 | 4.82E−02 |
| cg23702688 | ENSG00000135454 | 1.17E−02 | 0.2827 | 3.00E−02 |
| cg04244354 | ENSG00000221818 | 1.17E−02 | 0.4012 | 1.64E−03 |
| cg09940188 | ENSG00000132386 | 1.18E−02 | −0.4297 | 6.82E−04 |
| cg11957382 | ENSG00000013016 | 1.18E−02 | −0.2919 | 2.49E−02 |
| cg19703946 | ENSG00000155368 | 1.18E−02 | −0.2564 | 5.00E−02 |
| cg21201206 | ENSG00000068796 | 1.20E−02 | 0.3670 | 4.25E−03 |
| cg20803849 | ENSG00000162337 | 1.21E−02 | −0.3861 | 2.53E−03 |
| cg23704689 | ENSG00000111231 | 1.22E−02 | 0.2597 | 4.70E−02 |
| cg09251959 | ENSG00000084636 | 1.22E−02 | −0.3284 | 1.11E−02 |
| cg05976481 | ENSG00000257242 | 1.23E−02 | −0.5744 | 1.96E−06 |
| cg05206120 | ENSG00000100626 | 1.23E−02 | 0.3941 | 2.01E−03 |
| cg10531711 | ENSG00000030110 | 1.28E−02 | 0.2585 | 4.81E−02 |
| cg08697503 | ENSG00000135903 | 1.28E−02 | 0.4652 | 2.05E−04 |
| cg05273205 | ENSG00000221818 | 1.28E−02 | 0.2996 | 2.12E−02 |
| cg03143486 | ENSG00000164818 | 1.28E−02 | −0.3492 | 6.71E−03 |
| cg24051057 | ENSG00000091986 | 1.29E−02 | −0.4513 | 3.34E−04 |
| cg03906996 | ENSG00000108001 | 1.31E−02 | 0.2720 | 3.71E−02 |
| cg09807227 | ENSG00000141551 | 1.31E−02 | 0.2662 | 4.16E−02 |
| cg18204200 | ENSG00000198133 | 1.32E−02 | 0.3271 | 1.15E−02 |
| cg17619347 | ENSG00000087085 | 1.32E−02 | −0.3212 | 1.31E−02 |
| cg10630085 | ENSG00000054965 | 1.32E−02 | −0.3731 | 3.61E−03 |
| cg18274619 | ENSG00000074416 | 1.33E−02 | −0.3811 | 2.90E−03 |
| cg14870792 | ENSG00000151067 | 1.35E−02 | 0.5016 | 5.18E−05 |
| cg04657684 | ENSG00000110675 | 1.36E−02 | 0.4476 | 3.78E−04 |
| cg07873848 | ENSG00000163082 | 1.37E−02 | −0.5013 | 5.26E−05 |
| cg24855780 | ENSG00000149115 | 1.37E−02 | −0.3806 | 2.95E−03 |
| cg01417714 | ENSG00000176444 | 1.37E−02 | −0.2655 | 4.22E−02 |
| cg01417714 | ENSG00000177628 | 1.37E−02 | −0.2893 | 2.62E−02 |
| cg19120580 | ENSG00000174233 | 1.37E−02 | −0.2722 | 3.70E−02 |
| cg24531396 | ENSG00000198125 | 1.38E−02 | −0.4275 | 7.33E−04 |
| cg02329670 | ENSG00000258315 | 1.39E−02 | −0.3179 | 1.41E−02 |
| cg26391674 | ENSG00000088899 | 1.41E−02 | 0.3397 | 8.48E−03 |
| cg26115470 | ENSG00000182866 | 1.42E−02 | −0.2742 | 3.56E−02 |
| cg08250880 | ENSG00000150687 | 1.43E−02 | −0.2590 | 4.76E−02 |
| cg25008504 | ENSG00000162849 | 1.48E−02 | −0.3226 | 1.27E−02 |
| cg09087897 | ENSG00000070495 | 1.49E−02 | −0.3376 | 8.92E−03 |
| cg03217019 | ENSG00000198231 | 1.50E−02 | −0.3665 | 4.31E−03 |
| cg03217019 | ENSG00000087191 | 1.50E−02 | 0.2962 | 2.27E−02 |
| cg13066481 | ENSG00000065534 | 1.51E−02 | −0.3425 | 7.93E−03 |
| cg13066481 | ENSG00000239523 | 1.51E−02 | −0.2906 | 2.55E−02 |
| cg24358467 | ENSG00000196526 | 1.51E−02 | −0.3001 | 2.09E−02 |
| cg15172529 | ENSG00000188820 | 1.52E−02 | −0.3330 | 9.97E−03 |
| cg25365990 | ENSG00000122367 | 1.52E−02 | 0.5409 | 9.76E−06 |
| cg03341641 | ENSG00000128805 | 1.55E−02 | −0.2747 | 3.53E−02 |
| cg25350057 | ENSG00000116729 | 1.57E−02 | 0.2984 | 2.17E−02 |
| cg25350057 | ENSG00000232284 | 1.57E−02 | 0.2567 | 4.97E−02 |
| cg09427605 | ENSG00000151612 | 1.57E−02 | −0.4364 | 5.49E−04 |
| cg20735050 | ENSG00000166025 | 1.58E−02 | −0.4111 | 1.22E−03 |
| cg07319712 | ENSG00000099817 | 1.60E−02 | −0.4364 | 5.50E−04 |
| cg22478679 | ENSG00000150401 | 1.60E−02 | −0.5879 | 9.78E−07 |
| cg22478679 | ENSG00000153531 | 1.60E−02 | −0.5587 | 4.26E−06 |
| cg00823095 | ENSG00000076356 | 1.63E−02 | −0.4610 | 2.39E−04 |
| cg02762475 | ENSG00000113504 | 1.63E−02 | −0.2942 | 2.37E−02 |
| cg10281002 | ENSG00000255399 | 1.64E−02 | 0.4567 | 2.77E−04 |
| cg10281002 | ENSG00000089225 | 1.64E−02 | 0.3845 | 2.64E−03 |
| cg04398180 | ENSG00000150401 | 1.64E−02 | −0.5180 | 2.65E−05 |
| cg04398180 | ENSG00000153531 | 1.64E−02 | −0.5879 | 9.82E−07 |
| cg26477856 | ENSG00000065357 | 1.64E−02 | −0.7732 | 7.12E−13 |
| cg00182639 | ENSG00000255399 | 1.64E−02 | 0.3342 | 9.67E−03 |
| cg00149455 | ENSG00000226900 | 1.65E−02 | −0.3334 | 9.88E−03 |
| cg04460364 | ENSG00000133026 | 1.67E−02 | −0.5698 | 2.48E−06 |
| cg03877376 | ENSG00000255399 | 1.68E−02 | 0.4684 | 1.83E−04 |
| cg03877376 | ENSG00000089225 | 1.68E−02 | 0.4160 | 1.05E−03 |
| cg20388732 | ENSG00000126561 | 1.69E−02 | −0.4670 | 1.92E−04 |
| cg19666787 | ENSG00000108175 | 1.69E−02 | −0.3828 | 2.77E−03 |
| cg10035294 | ENSG00000135903 | 1.69E−02 | 0.6140 | 2.32E−07 |
| cg13112511 | ENSG00000113448 | 1.70E−02 | 0.5259 | 1.89E−05 |
| cg15567428 | ENSG00000019991 | 1.71E−02 | −0.5062 | 4.32E−05 |
| cg14279151 | ENSG00000096093 | 1.71E−02 | 0.3789 | 3.08E−03 |
| cg08842032 | ENSG00000049283 | 1.73E−02 | −0.5686 | 2.62E−06 |
| cg08842032 | ENSG00000006282 | 1.73E−02 | −0.3688 | 4.05E−03 |
| cg24088496 | ENSG00000184384 | 1.73E−02 | −0.3827 | 2.78E−03 |
| cg03861065 | ENSG00000138311 | 1.73E−02 | −0.3007 | 2.07E−02 |
| cg19578183 | ENSG00000136720 | 1.73E−02 | 0.3937 | 2.04E−03 |
| cg10594510 | ENSG00000113504 | 1.74E−02 | −0.5765 | 1.77E−06 |
| cg19576099 | ENSG00000057657 | 1.74E−02 | −0.3577 | 5.41E−03 |
| cg02337873 | ENSG00000175602 | 1.74E−02 | 0.4054 | 1.45E−03 |
| cg17285709 | ENSG00000072195 | 1.75E−02 | −0.2570 | 4.94E−02 |
| cg17285709 | ENSG00000175084 | 1.75E−02 | −0.3923 | 2.12E−03 |
| cg25735482 | ENSG00000257337 | 1.77E−02 | −0.3960 | 1.90E−03 |
| cg10299448 | ENSG00000007237 | 1.78E−02 | −0.3302 | 1.07E−02 |
| cg07313373 | ENSG00000162711 | 1.81E−02 | −0.4435 | 4.34E−04 |
| cg03487276 | ENSG00000184939 | 1.81E−02 | 0.3308 | 1.05E−02 |
| cg22804358 | ENSG00000099994 | 1.82E−02 | −0.2977 | 2.20E−02 |
| cg20469275 | ENSG00000064692 | 1.86E−02 | −0.4044 | 1.49E−03 |
| cg06510261 | ENSG00000174500 | 1.86E−02 | −0.5727 | 2.14E−06 |
| cg00790098 | ENSG00000135903 | 1.87E−02 | 0.5079 | 4.02E−05 |
| cg24698488 | ENSG00000157227 | 1.90E−02 | −0.5544 | 5.21E−06 |
| cg19156552 | ENSG00000169855 | 1.90E−02 | −0.3843 | 2.65E−03 |
| cg19236454 | ENSG00000079432 | 1.92E−02 | 0.3742 | 3.50E−03 |
| cg03755566 | ENSG00000107796 | 1.92E−02 | −0.4825 | 1.09E−04 |
| cg03755566 | ENSG00000138134 | 1.92E−02 | −0.3483 | 6.86E−03 |
| cg05431842 | ENSG00000169851 | 1.92E−02 | −0.6128 | 2.49E−07 |
| cg18885210 | ENSG00000074047 | 1.93E−02 | −0.3316 | 1.03E−02 |
| cg13925890 | ENSG00000156427 | 1.96E−02 | −0.7662 | 1.54E−12 |
| cg18232841 | ENSG00000089356 | 1.97E−02 | −0.3620 | 4.84E−03 |
| cg21777154 | ENSG00000116667 | 1.97E−02 | −0.4631 | 2.21E−04 |
| cg26495839 | ENSG00000267277 | 1.97E−02 | 0.2882 | 2.68E−02 |
| cg26495839 | ENSG00000187266 | 1.97E−02 | 0.3869 | 2.47E−03 |
| cg15058645 | ENSG00000115935 | 1.98E−02 | −0.3679 | 4.15E−03 |
| cg23729443 | ENSG00000160783 | 1.99E−02 | 0.2851 | 2.86E−02 |
| cg23729443 | ENSG00000198952 | 1.99E−02 | 0.4038 | 1.52E−03 |
| cg13066289 | ENSG00000176771 | 2.01E−02 | 0.2636 | 4.36E−02 |
| cg25401628 | ENSG00000196562 | 2.01E−02 | −0.5585 | 4.29E−06 |
| cg14137548 | ENSG00000165633 | 2.01E−02 | −0.2852 | 2.86E−02 |
| cg04968127 | ENSG00000072952 | 2.02E−02 | −0.2776 | 3.33E−02 |
| cg13832290 | ENSG00000137135 | 2.03E−02 | −0.2914 | 2.51E−02 |
| cg13832290 | ENSG00000137101 | 2.03E−02 | −0.5522 | 5.80E−06 |
| cg13832290 | ENSG00000137078 | 2.03E−02 | −0.6289 | 9.62E−08 |
| cg12146829 | ENSG00000113504 | 2.03E−02 | −0.3901 | 2.26E−03 |
| cg22164891 | ENSG00000171940 | 2.04E−02 | −0.4827 | 1.08E−04 |
| cg22164891 | ENSG00000197670 | 2.04E−02 | −0.4560 | 2.83E−04 |
| cg25597580 | ENSG00000183853 | 2.05E−02 | −0.3678 | 4.15E−03 |
| cg07266616 | ENSG00000134853 | 2.06E−02 | −0.5545 | 5.20E−06 |
| cg07266616 | ENSG00000145216 | 2.06E−02 | −0.3065 | 1.82E−02 |
| cg10864952 | ENSG00000169902 | 2.06E−02 | −0.2680 | 4.01E−02 |
| cg19116959 | ENSG00000151612 | 2.08E−02 | −0.4543 | 3.02E−04 |
| cg12978308 | ENSG00000140365 | 2.12E−02 | −0.3128 | 1.59E−02 |
| cg00142933 | ENSG00000072163 | 2.12E−02 | −0.3668 | 4.27E−03 |
| cg24159247 | ENSG00000150995 | 2.15E−02 | −0.2612 | 4.57E−02 |
| cg18548864 | ENSG00000144857 | 2.17E−02 | −0.3541 | 5.93E−03 |
| cg10612492 | ENSG00000106333 | 2.19E−02 | 0.3776 | 3.20E−03 |
| cg22571654 | ENSG00000144893 | 2.20E−02 | −0.2898 | 2.60E−02 |
| cg24884140 | ENSG00000108641 | 2.21E−02 | −0.4642 | 2.13E−04 |
| cg24144440 | ENSG00000092607 | 2.21E−02 | 0.4965 | 6.36E−05 |
| cg23916284 | ENSG00000168214 | 2.23E−02 | 0.3811 | 2.90E−03 |
| cg19854293 | ENSG00000113504 | 2.24E−02 | −0.5325 | 1.42E−05 |
| cg11195002 | ENSG00000090339 | 2.26E−02 | −0.3187 | 1.39E−02 |
| cg17497640 | ENSG00000138411 | 2.28E−02 | −0.3045 | 1.90E−02 |
| cg12616177 | ENSG00000196411 | 2.28E−02 | −0.3015 | 2.03E−02 |
| cg21147983 | ENSG00000175206 | 2.30E−02 | −0.4437 | 4.32E−04 |
| cg21147983 | ENSG00000242349 | 2.30E−02 | −0.4133 | 1.14E−03 |
| cg17645823 | ENSG00000255399 | 2.31E−02 | 0.5197 | 2.46E−05 |
| cg17645823 | ENSG00000089225 | 2.31E−02 | 0.3704 | 3.88E−03 |
| cg03611852 | ENSG00000161714 | 2.32E−02 | −0.3318 | 1.03E−02 |
| cg04254886 | ENSG00000165795 | 2.32E−02 | 0.3420 | 8.03E−03 |
| cg11592677 | ENSG00000182197 | 2.33E−02 | −0.2653 | 4.23E−02 |
| cg21980394 | ENSG00000185345 | 2.35E−02 | −0.2732 | 3.63E−02 |
| cg05021796 | ENSG00000250900 | 2.36E−02 | 0.3004 | 2.08E−02 |
| cg05021796 | ENSG00000146054 | 2.36E−02 | 0.2921 | 2.48E−02 |
| cg04171808 | ENSG00000026508 | 2.36E−02 | −0.7314 | 4.74E−11 |
| cg21907579 | ENSG00000255399 | 2.39E−02 | 0.3461 | 7.25E−03 |
| cg21296513 | ENSG00000149564 | 2.40E−02 | −0.3369 | 9.07E−03 |
| cg03110167 | ENSG00000167657 | 2.42E−02 | −0.2657 | 4.20E−02 |
| ch.5.2517577F | ENSG00000152377 | 2.43E−02 | −0.4141 | 1.11E−03 |
| cg13270873 | ENSG00000104447 | 2.46E−02 | −0.4685 | 1.83E−04 |
| cg02584488 | ENSG00000110429 | 2.46E−02 | −0.2862 | 2.80E−02 |
| cg22291922 | ENSG00000120093 | 2.47E−02 | −0.3886 | 2.35E−03 |
| cg22291922 | ENSG00000108511 | 2.47E−02 | −0.3536 | 6.01E−03 |
| cg01945840 | ENSG00000198125 | 2.47E−02 | −0.3554 | 5.74E−03 |
| cg16155393 | ENSG00000163827 | 2.48E−02 | 0.3509 | 6.43E−03 |
| cg07134316 | ENSG00000166888 | 2.48E−02 | −0.4051 | 1.46E−03 |
| cg21746120 | ENSG00000162337 | 2.48E−02 | −0.3573 | 5.47E−03 |
| cg16459103 | ENSG00000149243 | 2.48E−02 | 0.2587 | 4.78E−02 |
| cg10169241 | ENSG00000119559 | 2.50E−02 | −0.3584 | 5.32E−03 |
| cg10057528 | ENSG00000162585 | 2.50E−02 | −0.2945 | 2.36E−02 |
| cg10057528 | ENSG00000067606 | 2.50E−02 | −0.3051 | 1.88E−02 |
| cg15542639 | ENSG00000110218 | 2.51E−02 | −0.5029 | 4.92E−05 |
| cg14688579 | ENSG00000135903 | 2.53E−02 | 0.5289 | 1.66E−05 |
| cg24721309 | ENSG00000068650 | 2.54E−02 | −0.4419 | 4.59E−04 |
| cg14508696 | ENSG00000261801 | 2.58E−02 | 0.4797 | 1.21E−04 |
| cg25353436 | ENSG00000225383 | 2.59E−02 | −0.4240 | 8.19E−04 |
| cg05424970 | ENSG00000004059 | 2.60E−02 | 0.3573 | 5.47E−03 |
| cg09217157 | ENSG00000138185 | 2.64E−02 | −0.4552 | 2.92E−04 |
| cg17510121 | ENSG00000146197 | 2.68E−02 | −0.2722 | 3.70E−02 |
| cg16616918 | ENSG00000132386 | 2.70E−02 | −0.3658 | 4.38E−03 |
| cg04223553 | ENSG00000113196 | 2.71E−02 | −0.3010 | 2.05E−02 |
| cg04543156 | ENSG00000223820 | 2.72E−02 | 0.2898 | 2.60E−02 |
| cg18919660 | ENSG00000185070 | 2.74E−02 | −0.2662 | 4.16E−02 |
| cg13371883 | ENSG00000170485 | 2.74E−02 | −0.3877 | 2.41E−03 |
| cg27009545 | ENSG00000136404 | 2.74E−02 | −0.4472 | 3.84E−04 |
| cg04112126 | ENSG00000171798 | 2.75E−02 | −0.4520 | 3.26E−04 |
| cg23024136 | ENSG00000113196 | 2.76E−02 | −0.3278 | 1.13E−02 |
| cg10624914 | ENSG00000150401 | 2.77E−02 | −0.5535 | 5.44E−06 |
| cg10624914 | ENSG00000153531 | 2.77E−02 | −0.4908 | 7.93E−05 |
| cg03347450 | ENSG00000135447 | 2.80E−02 | −0.3173 | 1.43E−02 |
| cg08936706 | ENSG00000160808 | 2.80E−02 | −0.5436 | 8.62E−06 |
| cg22157087 | ENSG00000091831 | 2.81E−02 | −0.3459 | 7.30E−03 |
| cg08052292 | ENSG00000163947 | 2.86E−02 | −0.7347 | 3.50E−11 |
| cg11117099 | ENSG00000184922 | 2.86E−02 | −0.3892 | 2.32E−03 |
| cg06607384 | ENSG00000133943 | 2.91E−02 | −0.3035 | 1.94E−02 |
| cg15161225 | ENSG00000198125 | 2.91E−02 | −0.5306 | 1.54E−05 |
| cg00572843 | ENSG00000177374 | 2.92E−02 | 0.3733 | 3.59E−03 |
| cg00572843 | ENSG00000070366 | 2.92E−02 | 0.2618 | 4.52E−02 |
| cg00572843 | ENSG00000108963 | 2.92E−02 | 0.3448 | 7.49E−03 |
| cg02829688 | ENSG00000092607 | 2.93E−02 | 0.5381 | 1.11E−05 |
| cg16665041 | ENSG00000215251 | 2.95E−02 | 0.3460 | 7.27E−03 |
| cg16665041 | ENSG00000185019 | 2.95E−02 | −0.3407 | 8.28E−03 |
| cg20252015 | ENSG00000079739 | 2.95E−02 | −0.4698 | 1.74E−04 |
| cg12522898 | ENSG00000101019 | 2.96E−02 | 0.2967 | 2.25E−02 |
| cg03942871 | ENSG00000128606 | 2.97E−02 | −0.5892 | 9.15E−07 |
| cg03942871 | ENSG00000161040 | 2.97E−02 | −0.3555 | 5.73E−03 |
| cg21301805 | ENSG00000092607 | 2.99E−02 | 0.3454 | 7.38E−03 |
| cg19709585 | ENSG00000196878 | 3.00E−02 | −0.2702 | 3.85E−02 |
| cg17800788 | ENSG00000142794 | 3.00E−02 | −0.6104 | 2.85E−07 |
| cg23186333 | ENSG00000026508 | 3.01E−02 | −0.6854 | 2.12E−09 |
| cg02633609 | ENSG00000137809 | 3.02E−02 | 0.3866 | 2.49E−03 |
| cg10380643 | ENSG00000235098 | 3.02E−02 | −0.3547 | 5.85E−03 |
| cg10380643 | ENSG00000225285 | 3.02E−02 | −0.3155 | 1.49E−02 |
| cg15428496 | ENSG00000144677 | 3.04E−02 | −0.4569 | 2.75E−04 |
| cg17253785 | ENSG00000175073 | 3.06E−02 | −0.2686 | 3.97E−02 |
| cg17253785 | ENSG00000213865 | 3.06E−02 | −0.2584 | 4.81E−02 |
| cg26535547 | ENSG00000161654 | 3.06E−02 | 0.4037 | 1.52E−03 |
| cg08679180 | ENSG00000110237 | 3.10E−02 | −0.5172 | 2.73E−05 |
| cg03548463 | ENSG00000189339 | 3.17E−02 | −0.3736 | 3.56E−03 |
| cg21948564 | ENSG00000140506 | 3.18E−02 | −0.2803 | 3.15E−02 |
| cg24621972 | ENSG00000135903 | 3.19E−02 | 0.5522 | 5.78E−06 |
| cg07403350 | ENSG00000139826 | 3.21E−02 | −0.3363 | 9.22E−03 |
| cg07403350 | ENSG00000174405 | 3.21E−02 | −0.3392 | 8.58E−03 |
| cg02715006 | ENSG00000204956 | 3.21E−02 | 0.4467 | 3.90E−04 |
| cg00343747 | ENSG00000156011 | 3.23E−02 | −0.3931 | 2.07E−03 |
| cg06560379 | ENSG00000146232 | 3.25E−02 | −0.3137 | 1.55E−02 |
| cg22999025 | ENSG00000128487 | 3.28E−02 | −0.3023 | 2.00E−02 |
| cg13229360 | ENSG00000174705 | 3.29E−02 | −0.4048 | 1.47E−03 |
| cg16419756 | ENSG00000113504 | 3.32E−02 | −0.4504 | 3.45E−04 |
| cg24074783 | ENSG00000160746 | 3.32E−02 | −0.3316 | 1.03E−02 |
| cg24074783 | ENSG00000163788 | 3.32E−02 | 0.3956 | 1.93E−03 |
| cg00781839 | ENSG00000150401 | 3.33E−02 | −0.5065 | 4.25E−05 |
| cg04658601 | ENSG00000168993 | 3.34E−02 | −0.2743 | 3.55E−02 |
| cg17452384 | ENSG00000109339 | 3.34E−02 | −0.4443 | 4.23E−04 |
| cg00864012 | ENSG00000136478 | 3.36E−02 | −0.3269 | 1.15E−02 |
| cg21163444 | ENSG00000161642 | 3.37E−02 | 0.4381 | 5.20E−04 |
| cg24541550 | ENSG00000072952 | 3.38E−02 | −0.4374 | 5.33E−04 |
| cg21919599 | ENSG00000162711 | 3.39E−02 | −0.6395 | 4.97E−08 |
| cg15641364 | ENSG00000158710 | 3.40E−02 | −0.2990 | 2.14E−02 |
| cg04674421 | ENSG00000169181 | 3.41E−02 | −0.5540 | 5.32E−06 |
| cg01322214 | ENSG00000153790 | 3.46E−02 | −0.3622 | 4.82E−03 |
| cg24843609 | ENSG00000160783 | 3.47E−02 | 0.2761 | 3.43E−02 |
| cg04913078 | ENSG00000183091 | 3.49E−02 | 0.3058 | 1.85E−02 |
| cg24406898 | ENSG00000164692 | 3.52E−02 | −0.4267 | 7.52E−04 |
| cg23360190 | ENSG00000101331 | 3.55E−02 | 0.3708 | 3.84E−03 |
| cg07567308 | ENSG00000185019 | 3.59E−02 | −0.2922 | 2.47E−02 |
| cg02378006 | ENSG00000107731 | 3.62E−02 | −0.4740 | 1.49E−04 |
| cg23931734 | ENSG00000074410 | 3.62E−02 | −0.4075 | 1.36E−03 |
| cg02511723 | ENSG00000131711 | 3.62E−02 | −0.3364 | 9.18E−03 |
| cg14855841 | ENSG00000169248 | 3.66E−02 | −0.4632 | 2.21E−04 |
| cg14855841 | ENSG00000169245 | 3.66E−02 | −0.4714 | 1.64E−04 |
| cg03764585 | ENSG00000122176 | 3.68E−02 | −0.3760 | 3.34E−03 |
| cg24699895 | ENSG00000156515 | 3.68E−02 | −0.5237 | 2.07E−05 |
| cg10986043 | ENSG00000173991 | 3.68E−02 | −0.3297 | 1.08E−02 |
| cg26541218 | ENSG00000158683 | 3.70E−02 | 0.3105 | 1.67E−02 |
| cg06069861 | ENSG00000082641 | 3.72E−02 | 0.3280 | 1.12E−02 |
| cg16990168 | ENSG00000092607 | 3.73E−02 | 0.5532 | 5.52E−06 |
| cg06786153 | ENSG00000167202 | 3.73E−02 | −0.2866 | 2.78E−02 |
| cg05403316 | ENSG00000115310 | 3.74E−02 | −0.5191 | 2.53E−05 |
| cg06431025 | ENSG00000172554 | 3.75E−02 | 0.2589 | 4.77E−02 |
| cg25918166 | ENSG00000226674 | 3.76E−02 | −0.3505 | 6.49E−03 |
| cg08880369 | ENSG00000187535 | 3.79E−02 | −0.4697 | 1.75E−04 |
| cg08880369 | ENSG00000131634 | 3.79E−02 | −0.5386 | 1.08E−05 |
| cg10634619 | ENSG00000227372 | 3.79E−02 | −0.2649 | 4.26E−02 |
| cg21814178 | ENSG00000110934 | 3.84E−02 | −0.4844 | 1.01E−04 |
| cg00622552 | ENSG00000182950 | 3.85E−02 | −0.3225 | 1.27E−02 |
| cg00364758 | ENSG00000106483 | 3.89E−02 | −0.3827 | 2.78E−03 |
| cg00364758 | ENSG00000086289 | 3.89E−02 | −0.3219 | 1.29E−02 |
| cg15535174 | ENSG00000149639 | 3.89E−02 | −0.4765 | 1.36E−04 |
| cg01963906 | ENSG00000142765 | 3.91E−02 | 0.4417 | 4.61E−04 |
| cg14678583 | ENSG00000133250 | 3.98E−02 | −0.3321 | 1.02E−02 |
| cg09262100 | ENSG00000198752 | 3.98E−02 | −0.3500 | 6.59E−03 |
| cg09004195 | ENSG00000116106 | 3.99E−02 | −0.6464 | 3.20E−08 |
| cg22941573 | ENSG00000240849 | 4.01E−02 | 0.2647 | 4.27E−02 |
| cg09645291 | ENSG00000156113 | 4.02E−02 | 0.4013 | 1.63E−03 |
| cg08668662 | ENSG00000131044 | 4.04E−02 | −0.3480 | 6.92E−03 |
| cg00604356 | ENSG00000105851 | 4.04E−02 | −0.4268 | 7.49E−04 |
| cg05318210 | ENSG00000226674 | 4.05E−02 | −0.3984 | 1.78E−03 |
| cg22950111 | ENSG00000117020 | 4.05E−02 | −0.3001 | 2.09E−02 |
| cg15281283 | ENSG00000183486 | 4.06E−02 | −0.2939 | 2.39E−02 |
| cg15281283 | ENSG00000183844 | 4.06E−02 | −0.3477 | 6.97E−03 |
| cg02657611 | ENSG00000132773 | 4.09E−02 | 0.3586 | 5.29E−03 |
| cg02657611 | ENSG00000070759 | 4.09E−02 | 0.3824 | 2.80E−03 |
| cg11885555 | ENSG00000108604 | 4.12E−02 | −0.3415 | 8.12E−03 |
| cg26919014 | ENSG00000102996 | 4.13E−02 | −0.4081 | 1.33E−03 |
| cg02461363 | ENSG00000196932 | 4.16E−02 | −0.2667 | 4.11E−02 |
| cg02941085 | ENSG00000155093 | 4.18E−02 | 0.2786 | 3.26E−02 |
| cg05265258 | ENSG00000132256 | 4.19E−02 | −0.2655 | 4.21E−02 |
| cg10155522 | ENSG00000149218 | 4.19E−02 | −0.4401 | 4.87E−04 |
| cg23648809 | ENSG00000182873 | 4.24E−02 | 0.3623 | 4.80E−03 |
| cg23648809 | ENSG00000067606 | 4.24E−02 | 0.3187 | 1.39E−02 |
| cg04876424 | ENSG00000112183 | 4.29E−02 | −0.3403 | 8.36E−03 |
| cg17258195 | ENSG00000129009 | 4.30E−02 | −0.4775 | 1.31E−04 |
| cg13748794 | ENSG00000120254 | 4.31E−02 | 0.5559 | 4.86E−06 |
| cg25832796 | ENSG00000213983 | 4.32E−02 | 0.4088 | 1.31E−03 |
| cg19784382 | ENSG00000011451 | 4.33E−02 | 0.2577 | 4.87E−02 |
| cg19784382 | ENSG00000105122 | 4.33E−02 | 0.2580 | 4.85E−02 |
| cg13276580 | ENSG00000182022 | 4.34E−02 | −0.5118 | 3.43E−05 |
| cg26100986 | ENSG00000106333 | 4.34E−02 | 0.3263 | 1.17E−02 |
| cg07025312 | ENSG00000047578 | 4.35E−02 | −0.3805 | 2.95E−03 |
| cg06032021 | ENSG00000177791 | 4.38E−02 | −0.3848 | 2.62E−03 |
| cg24339032 | ENSG00000143850 | 4.43E−02 | 0.3663 | 4.33E−03 |
| cg09042277 | ENSG00000255399 | 4.44E−02 | 0.3090 | 1.73E−02 |
| cg06728055 | ENSG00000018408 | 4.47E−02 | −0.3296 | 1.08E−02 |
| cg13873733 | ENSG00000152795 | 4.50E−02 | −0.4559 | 2.85E−04 |
| cg13873733 | ENSG00000145293 | 4.50E−02 | −0.4414 | 4.66E−04 |
| cg02722596 | ENSG00000253910 | 4.54E−02 | 0.2653 | 4.23E−02 |
| cg01941219 | ENSG00000152767 | 4.55E−02 | −0.3618 | 4.87E−03 |
| cg21783442 | ENSG00000134375 | 4.56E−02 | 0.2724 | 3.68E−02 |
| cg11027217 | ENSG00000073331 | 4.57E−02 | 0.3148 | 1.52E−02 |
| cg03603260 | ENSG00000197622 | 4.58E−02 | 0.4160 | 1.05E−03 |
| cg03603260 | ENSG00000143443 | 4.58E−02 | −0.3612 | 4.95E−03 |
| cg23647640 | ENSG00000184489 | 4.61E−02 | −0.3413 | 8.15E−03 |
| cg01709312 | ENSG00000150593 | 4.61E−02 | −0.5085 | 3.93E−05 |
| cg19542542 | ENSG00000163697 | 4.65E−02 | −0.3956 | 1.93E−03 |
| cg22060817 | ENSG00000135547 | 4.69E−02 | −0.4038 | 1.51E−03 |
| cg08942939 | ENSG00000092607 | 4.72E−02 | 0.4411 | 4.71E−04 |
| cg04738151 | ENSG00000198812 | 4.74E−02 | −0.2851 | 2.86E−02 |
| cg18619300 | ENSG00000134321 | 4.74E−02 | −0.2691 | 3.93E−02 |
| cg16202734 | ENSG00000067191 | 4.75E−02 | −0.2700 | 3.86E−02 |
| cg27355006 | ENSG00000150281 | 4.78E−02 | 0.3889 | 2.33E−03 |
| cg14637411 | ENSG00000053918 | 4.79E−02 | −0.2991 | 2.14E−02 |
| cg16003601 | ENSG00000105357 | 4.79E−02 | −0.2848 | 2.88E−02 |
| cg21990700 | ENSG00000139178 | 4.80E−02 | −0.7204 | 1.26E−10 |
| cg21990700 | ENSG00000205885 | 4.80E−02 | −0.6747 | 4.65E−09 |
| cg16016960 | ENSG00000132561 | 4.80E−02 | −0.6237 | 1.31E−07 |
| cg00589850 | ENSG00000253767 | 4.80E−02 | 0.5045 | 4.62E−05 |
| cg00589850 | ENSG00000204956 | 4.80E−02 | 0.4285 | 7.11E−04 |
| cg00589850 | ENSG00000253537 | 4.80E−02 | 0.3184 | 1.40E−02 |
| cg00589850 | ENSG00000253731 | 4.80E−02 | 0.4298 | 6.80E−04 |
| cg00589850 | ENSG00000253910 | 4.80E−02 | 0.4632 | 2.20E−04 |
| cg00589850 | ENSG00000253485 | 4.80E−02 | 0.4167 | 1.03E−03 |
| cg00589850 | ENSG00000262576 | 4.80E−02 | 0.3810 | 2.91E−03 |
| cg00589850 | ENSG00000253873 | 4.80E−02 | 0.3409 | 8.24E−03 |
| cg00589850 | ENSG00000262209 | 4.80E−02 | 0.4029 | 1.56E−03 |
| cg00589850 | ENSG00000253953 | 4.80E−02 | 0.2882 | 2.69E−02 |
| cg00589850 | ENSG00000242419 | 4.80E−02 | 0.2712 | 3.78E−02 |
| cg00589850 | ENSG00000254245 | 4.80E−02 | 0.3041 | 1.92E−02 |
| cg00589850 | ENSG00000253846 | 4.80E−02 | 0.2783 | 3.28E−02 |
| cg00589850 | ENSG00000254221 | 4.80E−02 | 0.3212 | 1.31E−02 |
| cg00589850 | ENSG00000253305 | 4.80E−02 | 0.3182 | 1.40E−02 |
| cg02696327 | ENSG00000102924 | 4.80E−02 | 0.4802 | 1.19E−04 |
| cg06917231 | ENSG00000197062 | 4.82E−02 | −0.2939 | 2.39E−02 |
| cg18108818 | ENSG00000128606 | 4.82E−02 | −0.2806 | 3.14E−02 |
| cg12178237 | ENSG00000172915 | 4.84E−02 | 0.4312 | 6.51E−04 |
| cg09430976 | ENSG00000221818 | 4.86E−02 | 0.2895 | 2.62E−02 |
| cg08771114 | ENSG00000184956 | 4.87E−02 | −0.2989 | 2.15E−02 |
| cg13654836 | ENSG00000153944 | 4.88E−02 | −0.2828 | 3.00E−02 |
| cg23009419 | ENSG00000241186 | 4.89E−02 | −0.3829 | 2.76E−03 |
| cg07768268 | ENSG00000090565 | 4.91E−02 | 0.2793 | 3.22E−02 |
| cg13054523 | ENSG00000261888 | 4.95E−02 | 0.3581 | 5.35E−03 |
| cg19489885 | ENSG00000087116 | 4.97E−02 | −0.3751 | 3.42E−03 |
| cg05940231 | ENSG00000092607 | 4.97E−02 | 0.4053 | 1.45E−03 |
| cg06595154 | ENSG00000072952 | 4.98E−02 | −0.4303 | 6.70E−04 |
| cg00203284 | ENSG00000186564 | 5.00E−02 | 0.4212 | 8.94E−04 |

Nominal p-values for Correlation. For DCM association, adjustment for gender, age and PCA.

### Results

### Epigenome-Wide Association Study of DCM

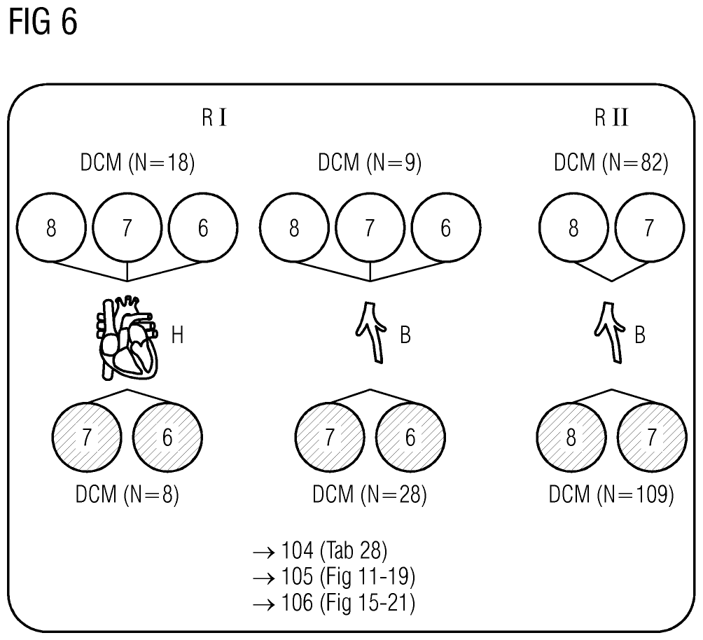

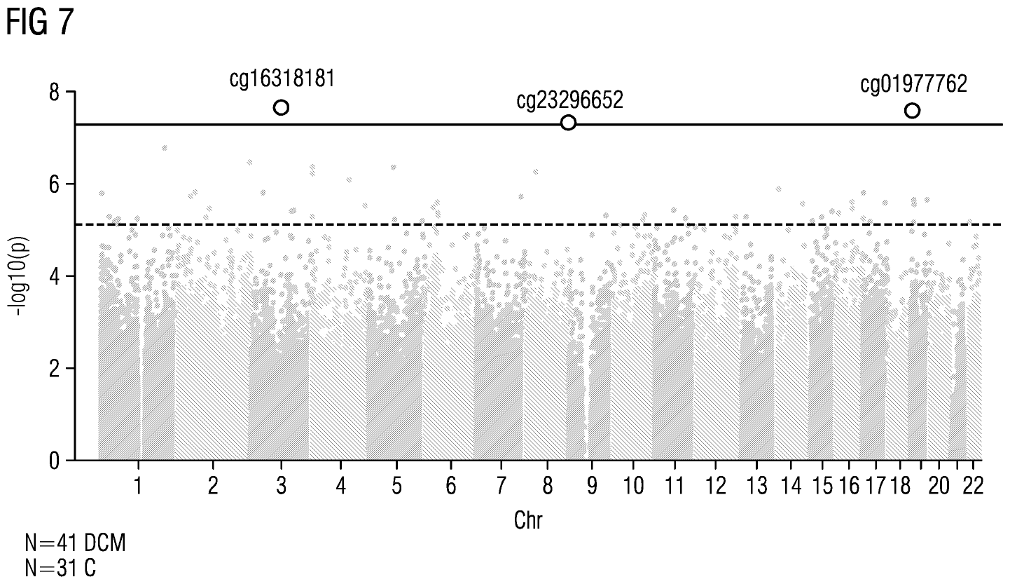

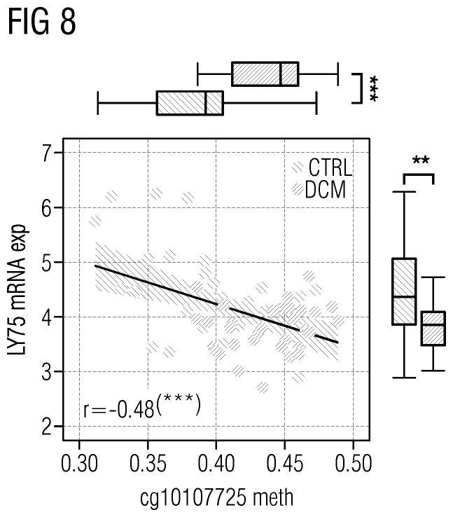

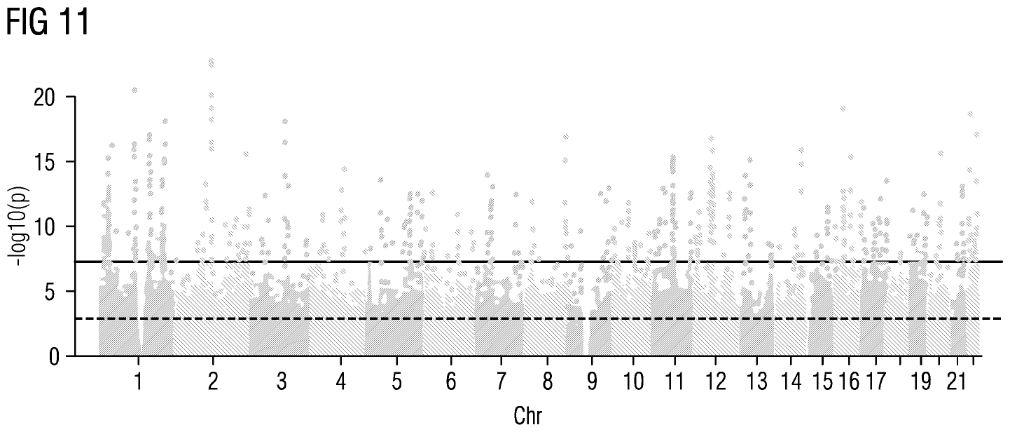

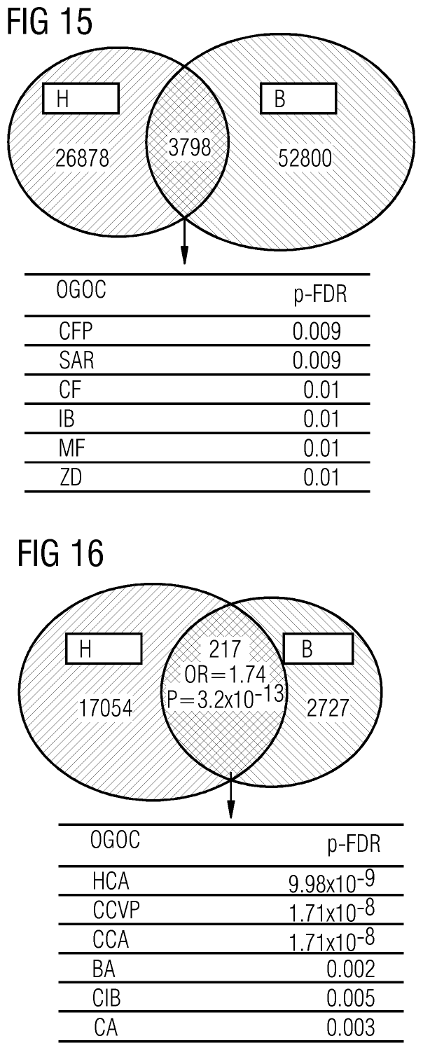

For the inclusion in this study, it was required that patients with systolic dysfunction and suspicion for DCM underwent extensive clinical phenotyping. Excluded were all patients who had hints for secondary causes of DCM from the detailed clinical work-up (see Materials and Methods section). A total of n=135 patients were included in the study. Since we only were interested in complete datasets and sufficient cardiac biomaterial as left-over, we excluded 94 individuals. In the final core cohort, n=41 patients for whom we were able to generate high quality DNA methylation profiles from heart tissue and peripheral blood were used in the screening stage of this study. None of these patients or controls did overlap with previous studies on DNA methylation (Haas J, et al., Alterations in cardiac DNA methylation in human dilated cardiomyopathy. EMBO Mol Med. 2013; 5:413-29). The mean age of patients was 54.1±12.3 and 63% were in early NYHA stages. As such, the median NT-proBNP was 812 ng/l, see Table 25. As control samples, we used left-ventricular biopsies from 31 patients free of heart failure with regular systolic and diastolic heart function who underwent routine left-heart myocardial biopsy after receiving heart transplantation, see Table 26. For an overview on patients, controls and molecular phenotyping, please see FIGS. 5 and 6, which show an overview of the study cohort in the multi-omics screening stage. FIG. 5 shows therein the screening in an abstract way, wherein N=41 for DCM. RNA 6, methylation 7, phenotype 8, biomarkers 9, and genome 10 have been determined for heart tissue H and blood B, respectively, as well as for HTX controls HTX, wherein N=31, and for clinical controls CC, wherein N=31. These data were used for epigenome-wide association study 100, as also shown in FIG. 7, identification of heart failure associated epigenetic patterns 101, as also shown in FIGS. 8-10, epigenetic regulation of cardiac RNA transcription 102, as also shown in FIGS. 11-14, and identification of conserved epigenetic patterns, as also shown in FIGS. 15-19. FIG. 6 shows data for a replication experiment R I with DCM (N=18) for heart tissue H and DCM (N=9) for blood B, wherein again RNA 6, methylation 7 and phenotype 8 were determined, as well as for healthy controls HC with N=8 for H and N=28 for B. In a replication experiment R II shown in FIG. 6 as well, DCM was N=82 and HC was N=109 for blood B, wherein methylation 7 and phenotype 8 were determined. These experiments enabled a validation of epigenome-wide association loci 104, as also shown in Table 28, a validation of DCM and mRNA associated methylation signatures 105, as also shown in FIGS. 11-19, and a validation of potential methylation biomarkers 106, as also shown in FIGS. 15-21.

After performing data quality control and normalization, we calculated genome-wide associations for each CpG site. Genomes were prima vista excluded from the analysis. To adjust for potential epigenomic inflation, we performed principal component (PC) analysis on methylation measurements and identified PCs, which were associated with confounders (methodological confounders as batch effects and biological confounders such as medication; FDR 0.05), see Tables 21 and 22. Dysregulated methylation sites were identified by linear modelling and moderated t-tests including age, gender as well as the identified principal components as covariates (Meder B, et al., Influence of the confounding factors age and sex on microRNA profiles from peripheral blood. Clin Chem. 2014; 60:1200-8).

From 485,000 methylation sites, 394,247 passed QC in myocardial tissue and blood. Genotype-associated methylation changes were excluded. 42,745 CpG-sites (9.5%) were found differentially methylated (raw-p≤0.05) in left-ventricle myocardium when comparing DCM vs. controls (33,396 of them being in 10 kb windows around annotated genes with expression in the cardiac tissue). The ratio of hypo-methylated vs hypermethylated CpG sites was 0.92. In blood samples, 35,566 (9%) were associated with DCM (raw p≤0,05; 28,153 being in a 10 kb window of annotated genes).

FIG. 7 shows a Manhattan plot of the epigenome-wide association study for Dilated Cardiomyopathy, showing an epigenome-wide association scan in cardiac tissue. Minus log 10 p-values are shown for single CpGs that passed the quality control criteria for the screening cohort. They are plotted against the chromosomes Chr on the x-axis. Probability values were based on linear modelling and moderated t-tests including age, gender and PCs as covariates. The solid line indicates the epigenome-wide significance level of p=5×10-8 and the dotted line indicates the false discovery (FDR) significance threshold of p=0.05. In the plot, N is 41 for DCM and N is 31 for controls C.

As summarized in the Manhattan plot in FIG. 7, after correcting for multiple testing we find 59 CpGs to be significantly differentially methylated in the myocardium of DCM patients (FDR-corrected p≤0.05; dotted line), with 30 sites that were hypomethylated and 29 sites hypermethylated in DCM. The delta of the methylation difference for FDR significant sites was in the median 14.34% (2.75%-69.9%). With the most stringent cut-off, we find 3 epigenome-wide significant loci with p-value 5×10-8 (solid line). The first of these loci (cg16318181, p=2.3×10-8) is on Chromosome 3, position 99,717,882. It is located within the gene body of CMSS1, the 5′UTR region of FILIP1L and part of the promoter region of miR-548G (within 1500 bp upstream of the transcription starting site). The second locus (cg01977762, p=2.8×10-8) is located on chromosome 19, position 4,909,193. It is within the promoter region of UHRF1 and part of a CpG island hr19:4,909,262-4,910,256. The third locus (cg23296652, p=4.8×10-8) is on chromosome 8, position 142,852,938 and not located near any known gene within a range of 10,000 bp.

To replicate these findings, we epigenotyped DNA from n=18 independent DCM patients and n=8 previously healthy control individuals that were casualties of roadside accidents. To the best of our knowledge, these control individuals were free of any heart condition and did not take regular medication. As shown in Table 35, we could successfully replicate 27 of the 59 loci (46%) in the independent cohorts. The most significant hit from the screening stage (cg16318181) could also be validated (replication p=0.004), resulting in a combined Fisher's p=2.23×10-09. In total, 5 hits superseded stringent genome-wide significance in the combined analysis.

TABLE 35
Replicated DNA methylation sites.

| | | Genes within 10 kb | Discovery | Replication | Fisher's combined |
|---|---|---|---|---|---|
| CpG | Chr | & cardiac expression | p-value | p-value | p-value |
| cg16318181 | 3 | FILIP1L; CMSS1 | 2.31728E−08 | 0.003988992 | 2.22813E−09 |
| cg25838968 | 1 | PLXNA2 | 1.62572E−07 | 0.000191836 | 7.85636E−10 |
| cg01726792 | 14 | NDRG2; TPPP2; RNASE7 | 1.31022E−06 | 0.000818940 | 2.32333E−08 |
| cg05978306 | 17 | MYO1C; CRK | 1.54725E−06 | 0.001279220 | 4.16450E−08 |
| cg18251389 | 7 | — | 1.83860E−06 | 0.012516509 | 4.27745E−07 |
| cg00586700 | 19 | FCGRT | 2.13918E−06 | 0.010759738 | 4.27818E−07 |
| cg18601596 | 6 | KCNK17 | 2.44359E−06 | 0.022232480 | 9.63125E−07 |
| cg03426023 | 16 | IRX5; CRNDE | 2.47814E−06 | 0.044349724 | 1.87098E−06 |
| cg11763830 | 17 | TTYH2 | 2.48453E−06 | 0.040090963 | 1.70547E−06 |
| cg24415066 | 4 | HAND2; HAND2-AS1 | 2.95582E−06 | 0.044796249 | 2.22943E−06 |
| cg17912835 | 2 | POU3F3 | 3.51740E−06 | 0.020237071 | 1.24270E−06 |
| cg19567891 | 15 | LINC00925 | 3.93465E−06 | 0.021478299 | 1.46087E−06 |
| ch.16.406779R | 16 | CLEC16A | 4.25310E−06 | 0.022104431 | 1.61512E−06 |
| cg17291767 | 6 | TRERF1 | 4.35941E−06 | 0.001627922 | 1.40258E−07 |
| cg02581963 | 10 | LINC00263; SCD | 4.55249E−06 | 0.010207249 | 8.31064E−07 |
| cg17399647 | 6 | TRERF1 | 4.67027E−06 | 0.007516221 | 6.37642E−07 |
| cg14523204 | 9 | RGS3 | 4.73687E−06 | 0.000876207 | 8.42549E−08 |
| cg24366665 | 13 | — | 5.19845E−06 | 0.003078146 | 3.03239E−07 |
| cg19194167 | 15 | CGNL1 | 5.21526E−06 | 0.019165689 | 1.71107E−06 |
| cg01294686 | 1 | CEP85; UBXN11; 3BGRL3 | 5.24878E−06 | 0.020249042 | 1.81288E−06 |
| cg08755532 | 2 | KCNIP3 | 5.49783E−06 | 0.015581898 | 1.47970E−06 |
| ch.1.117057666F | 1 | — | 5.53713E−06 | 0.005718672 | 5.78458E−07 |
| cg14504418 | 11 | BIRC3 | 5.54343E−06 | 0.035203372 | 3.21008E−06 |
| cg19683073 | 5 | SERINC5 | 5.83025E−06 | 0.003635288 | 3.95694E−07 |
| cg26941823 | 5 | STK10 | 6.21768E−06 | 0.009873932 | 1.08088E−06 |
| cg08281084 | 15 | HERC2 | 6.27797E−06 | 0.040257891 | 4.09205E−06 |
| cg16254946 | 1 | GLIS1 | 7.42353E−06 | 3.56842E−06 | 6.71640E−10 |

### Conserved DNA Methylation Sites in Heart Failure

In previous studies, mainly low-resolution approaches or very small cohorts were used to identify DNA methylation patterns for DCM and/or heart failure. Hence, to see if these findings can be reproduced in the current study, we compared methylation changes from the available previous studies (34 loci) and the current dataset. Since the methods varied largely and CpGs were not uniformly measured in the former studies, we used simes p-value aggregation of our dataset for the loci described previously. Using a cutoff of p≤0.05, we could replicate DNA methylation changes in the same direction in the genes LY75, PTGES, CTNNAL1, TNFSF14, MRPL16, KIF17, see Table 36 (Haas J, et al., Alterations in cardiac DNA methylation in human dilated cardiomyopathy. EMBO Mol Med. 2013; 5:413-29; Koczor C A, et al., Thymidine kinase and mtDNA depletion in human cardiomyopathy: epigenetic and translational evidence for energy starvation. Physiol Genomics. 2013; 45:590-6; Movassagh M, et al., Differential DNA methylation correlates with differential expression of angiogenic factors in human heart failure. PLoS One. 2010; 5:e8564; Garagnani P, et al., Methylation of ELOVL2 gene as a new epigenetic marker of age. Aging Cell. 2012; 11:1132-4), which supports the fact that heart failure is associated with certain defined, robust DNA methylation patterns. From all replicated loci, the LY75 methylation pattern showed the highest significance (simes p=0.002).

TABLE 36
Replication of DNA gene methylation from previous studies.

| | | Methylation in | |
|---|---|---|---|
| Gene | Reference | DCM/HF | p-value |
| LY75 | Haas et al. 2013 | Hyper-methylation | 0.0006 |
| PTGES | Koczor et al. 2013 | Hypo-methylation | 0.0028 |
| CTNNAL1 | Haas et al. 2013 | Hypo-methylation | 0.0099 |
| TNFSF14 | Koczor et al. 2013 | Hyper-methylation | 0.0100 |
| MRPL16 | Koczor et al. 2013 | Hypo-methylation | 0.0274 |
| KIF17 | Koczor et al. 2013 | Hyper-methylation | 0.0471 |

DCM = Dilated Cardiomyopathie; HF = heart failure.

Besides confirming hypermethylation of the LY75 gene locus, we also replicated the associated downregulation of LY75 expression levels in DCM, as seen in FIG. 8. FIG. 8 therein shows the methylation and expression of LY75 in myocardial/cardiac tissue. The diagram shows the correlation of cg10107725 in the promoter region and LY75 expression levels. Plotted is the LY75 mRNA expression (LY75 mRNA exp) on the y-axis versus cg10107725 methylation beta (cg10107725 meth) on the x-axis, with values plotted for DCM and control (CTRL). As for LY75, we could find a significant correlation between DNA methylation and mRNA expression, which underlines the regulatory role of the epigenetic code in the heart (*=p≤0.05, **=p≤0.01, ***=p≤0.001).

As for the successful replication of previous findings in tissue, we successfully replicated known age-dependent patterns in CpG islands within ELOVL2, FHL2 and PENK (Garagnani et al., 2012) in the DNA derived from whole peripheral blood samples of our cohort (simes significance level <10-14).

### Detection of Methylation Patterns in DCM

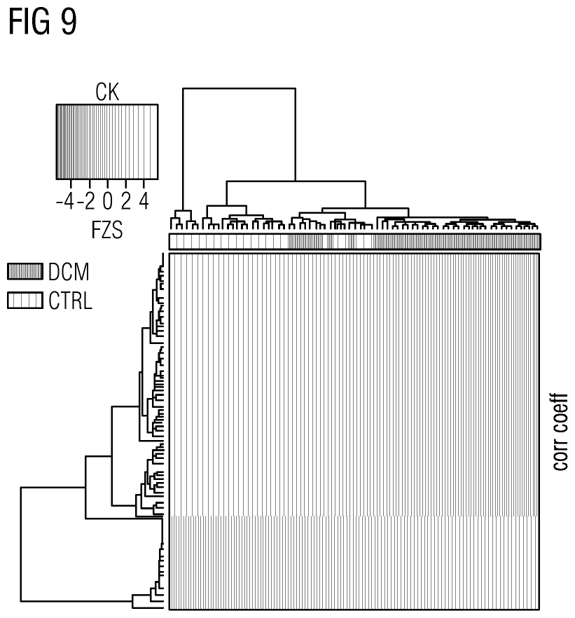

In unsupervised cluster analysis, showing DNA methylation in cardiac tissue—as seen in FIG. 9, we found that DNA methylation differences are able to cluster DCM patients and controls, underlining a disturbance or reprogramming of DNA methylation in heart failure. FIG. 9 therein shows cluster analysis in myocardial tissue, showing a correlation coefficient with a certain color key CK for a flow z-score FZS. As shown, cases and controls group very well together, indicating conserved methylation changes in DCM.

To test for possible functional methylation patterns, we first performed overrepresentation analysis for genome-wide transcription- and enhancer factor binding sites (Mathelier A, et al., JASPAR 2016: a major expansion and update of the open-access database of transcription factor binding profiles. Nucleic Acids Res. 2016; 44:D110-5) and their potential affection by DNA methylation. From 158,979 CpGs within annotated sequence motifs, we detected 4 motifs significantly associated with methylation alterations in DCM (FDR-p≤0.05), as shown in Table 23. Of interest, three of the motif-binding factors (Smad2, Smad4 and Bmal1) are known to be involved in cardiac remodeling during DCM and heart failure (Lefta M, Campbell K S, Feng H Z, Jin J P and Esser K A. Development of dilated cardiomyopathy in Bmal1-deficient mice. Am J Physiol Heart Circ Physiol. 2012; 303:H475-85).

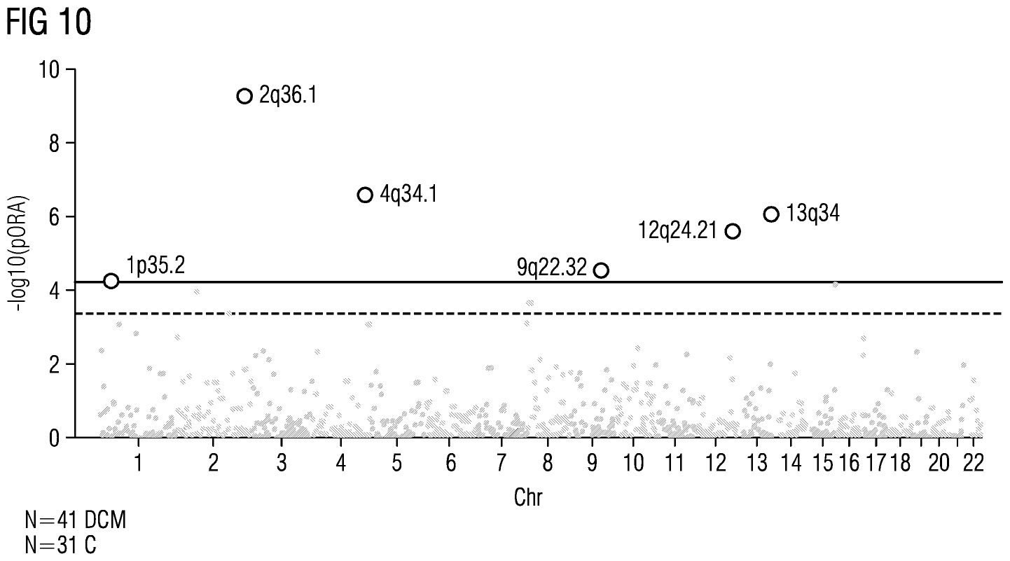

There is ample evidence that larger stretches of DNA methylation cluster together and exhibit repression of cis-regulatory elements. Hence, we carried out an overrepresentation analysis for clustering of differentially methylated sites at raw-p≤0.05 in specific chromosomal bands and found 6 regions to be significantly differentially methylated in DCM (Bonferroni level p≤0.05), as seen in FIG. 10. FIG. 10 therein shows a Chromosome Band Overrepresentation Analysis plot, particularly an epigenome-wide association chromosome band scan in cardiac tissue. Minus log 10 p-values are shown for overrepresentation analysis (pORA) based on chromosome bands in the screening cohort. The solid line indicates the Bonferroni-corrected significance level of 0.05 and the dotted line indicates the FDR-corrected significance threshold of p=0.05.

These regions host noticeable numbers of genes associated with cardiac development, heart function and cardiomyopathy. As an example, we found the gene locus 12q24.21 to be differentially methylated in DCM (78 out of 425 methylation sites show association with DCM at raw-p≤0.05, fisher's exact p=2×10-6). The 12q24.21 locus is harbouring several genes that have previously been linked to cardiomyopathies or cardiac development. One of the genes is TBX5, coding for a protein that is part of the T-Box family, known to be implicated in embryonic development and cardiogenesis (Papaioannou V E. The T-box gene family: emerging roles in development, stem cells and cancer. Development. 2014; 141:3819-33). Mutations in TBX5 could lately been shown in patients suffering from familial, as well as sporadic dilated cardiomyopathy (Zhou W, Zhao L, Jiang J Q, Jiang W F, Yang Y Q and Qiu X B. A novel TBX5 loss-of-function mutation associated with sporadic dilated cardiomyopathy. Int J Mol Med. 2015; 36:282-8). Another gene within this locus is MED13L, which is part of the Mediator complex family, which is also known to be involved in cardiovascular disease (Schiano C, Casamassimi A, Vietri M T, Rienzo M and Napoli C. The roles of mediator complex in cardiovascular diseases. Biochim Biophys Acta. 2014; 1839:444-51) and early heart development, leading to a variety of inborn cardiac abnormalities when disturbed (Samanek M. Congenital heart malformations: prevalence, severity, survival, and quality of life. Cardiol Young. 2000; 10:179-85). Additionally, we find the MYL2 gene within close vicinity to the 12q24.21 locus, which is coding for the ventricular regulatory Myosin Light Chain. It has an essential role during early embryonic cardiac development and represents one of the earliest markers of ventricular specification. Mutations in MYL2 are furthermore associated with Dilated and Hypertrophic Cardiomyopathy (O'Brien T X, Lee K J and Chien K R. Positional specification of ventricular myosin light chain 2 expression in the primitive murine heart tube. Proc Natl Acad Sci USA. 1993; 90:5157-61). Together, we found evidence for coordinated DNA methylation patterning in key cardiac developmental genomic regions.

### Impact of Differential DNA Methylation on Cardiac Gene Expression

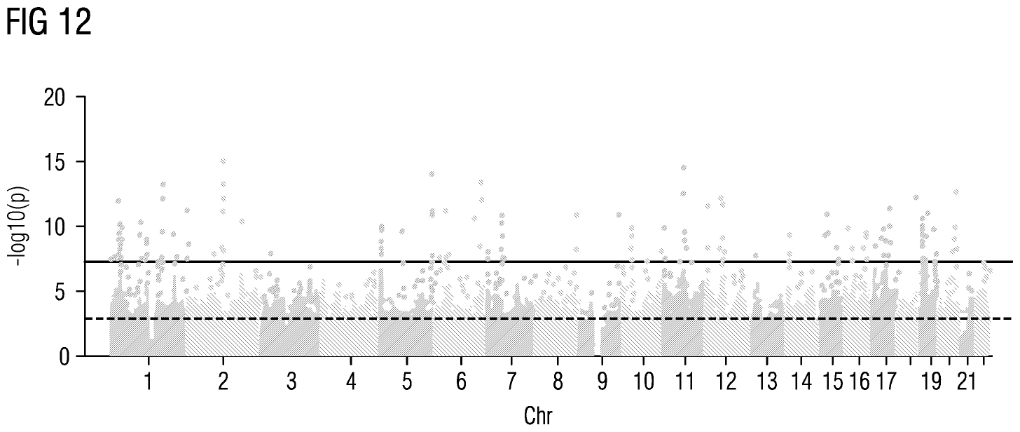

To test if the observed alterations in the degree of DNA methylation also act on global gene expression, we performed poly-A enriched mRNA sequencing in isolated RNA from the same biopsies that were taken for the methylation analysis in our discovery cohort. To link expression and DNA methylation, we performed meteQTL-analysis and identified a wide range of DNA methylation sites acting on cardiac transcription across the entire genome, as shown in FIGS. 11 and 12 FIGS. 11 and 12 depict Manhattan plots for methylation loci associated with down- and upregulation of mRNA expression in cardiac tissue, with FIG. 11 showing an epigenome-wide methQTL scan for negative association in cardiac tissue, and FIG. 12 showing an epigenome-wide methQTL scan for positive association in cardiac tissue. The solid line indicates the epigenome-wide significance level of p=5×10-8 and the dotted line indicates the (FDR) significance threshold of FDR-p=0.05.

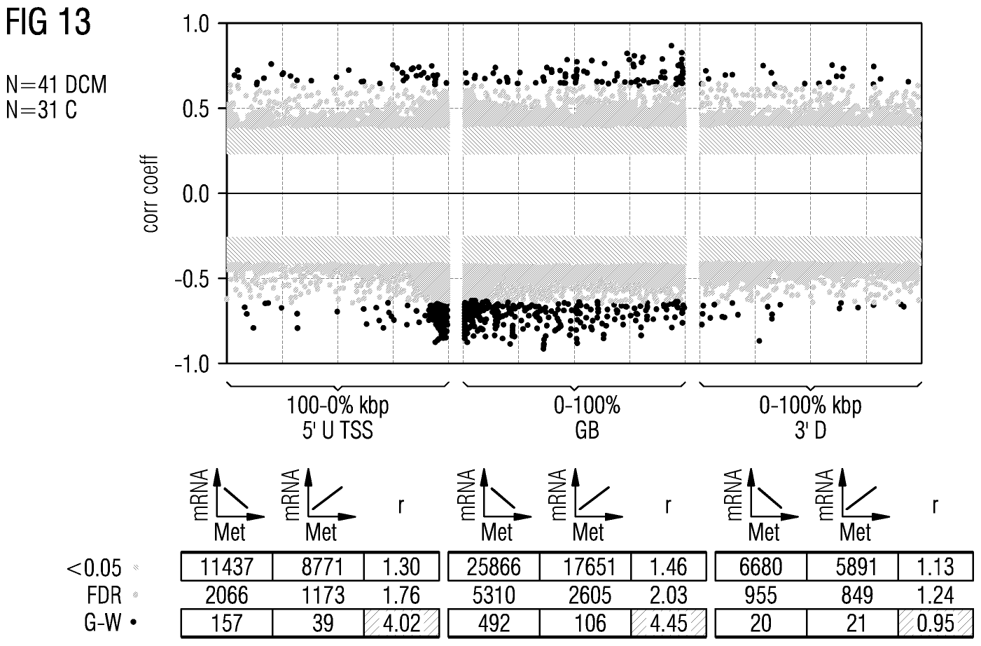

DNA hypermethylation within in the promoter region and the vicinity of transcription start-sites was found to be strongly associated with transcriptional downregulation and hypomethylation with upregulation. For 3′ downstream regions as well as towards the end of the gene body we find an equal ratio of positive and negative correlation between methylation status and gene expression levels, as seen in FIG. 13. FIG. 13 shows a correlation analysis of DNA methylation and mRNA expression depending on the position of the CpG relative to the associated gene, particularly methylation-mRNA association in cardiac tissue. Plotted is the correlation coefficient for—from left to right 100-0% 10 kbp for 5′ upstream TSS (5′ U TSS), 0-100% for gene body (GB), and 0-100% 10 kbp for 3′ downstream (3′ D) CpGs with an uncorrected p-value <0.05 are depicted in grey hatched from top left to bottom right, FDR corrected <0.05 are dark grey hatched from top right to bottom left, and genome-wide significant ones are black. Also shown are the ratios of mRNA and methylation Met for upslope and downslope as well as the ratio r thereof.

From the 33,396 CpG-sites found to be differentially methylated (raw-p≤0.05) in DCM and within 10 kb of genes expressed in the cardiac tissue, 8,420 CpGs were also significantly associated with gene expression in the discovery cohort (raw-p≤0.05). The observed overlap between DNA methylation and mRNA abundancy is far higher than expected by chance (Fisher exact p=7×10-67), which indicates that DNA methylation has a considerably strong functional impact on gene transcription in the heart.

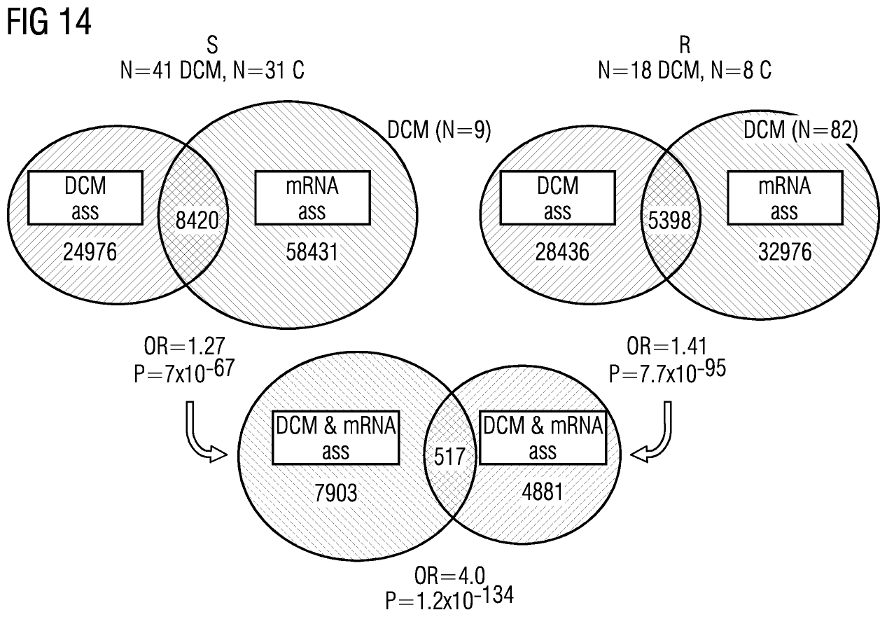

To dissect the role of these changes during DCM and also take into account the most valid candidates, we performed an independent validation study. The controls of the validation cohort, which were casualties of road accidents, were to the best of our knowledge free of any heart condition and did not take medication. To not only eliminate potential biological confounders, we chose a different mRNA sequencing protocol using random primers instead of poly-A enrichment. Samples were sequenced to a median paired-end read count of 37.17 million and mapping percentages were in the median 88.09. By combining these two independent study cohorts, we could generate a set of high confidence DNA methylation and expression sites for DCM. In detail, 517 different CpGs were directionally replicated on two levels (Fisher exact p=1.2×10-134), (i) to be associated with DCM and (ii) to act on mRNA transcription, as can be seen from FIG. 14 and Table 34. FIG. 14 therein shows a diagram of DNA methylation sites with DCM and/or RNA association in myocardial tissue. Shown on the left is the screening S of cardiac tissue with N=41 for DCM and N=31 for control C, and on the right the replication R of cardiac tissue with N=18 for DCM and N=8 for control C. For each DCM association DCM ass and mRNA association mRNA ass are shown, as well as the overlap, and at the bottom the overlap of the respective overlaps for DCM & mRNA association DCM & mRNA ass. The diagram indicates cardiac methylation sites that are linked to DCM and/or are associated with cardiac gene expression in the discovery and the replication cohorts for which both DNA methylation and mRNA expression where available (all at nominal p-value <0.05). The 517 replicated CpGs are associated with DCM and mRNA expression (p=1.2×10-134).

As shown by gene ontology overrepresentation analysis, the host genes of the methylation sites are mostly related to pathways linked to cardiac development and muscle function, as also shown in Table 24, further indicating that coordination of the expression of important functional genes in the course of (early) heart failure is driven by DNA methylation.

Two of the genome-wide significantly replicated methylation sites (see Table 35) were found to also be associated with expression of neighboring genes in the discovery and verification cohorts. Methylation status of cg25838968 was associated with PLXNA2 expression level (combined p=0.02), which is also differentially expressed in DCM (combined p=3×10-5). Methylation status of cg14523204 is associated with RGS3 (Regulator Of G-Protein Signaling 3) expression (combined p=0.0004), which we found to be differentially expressed in DCM as well (combined p=0.02).

### Conservation of DNA Methylation Patterns Across Tissues

The methylation and expression analyses resolved interesting new loci potentially involved in the pathogenesis of heart failure. As shown above, we for instance could replicate the strong association of myocardial LY75 methylation and expression with DCM. However, LY75 methylation is different in peripheral blood, hampering the immediate use as peripheral blood marker.

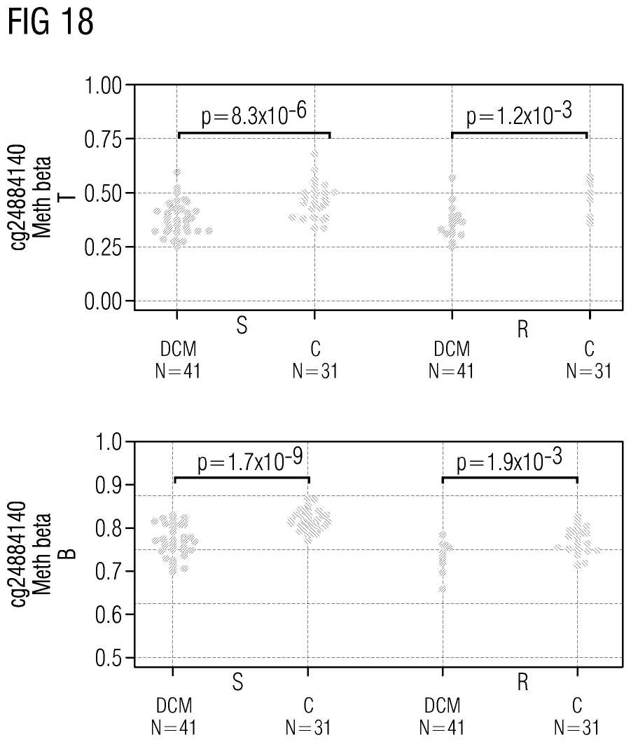

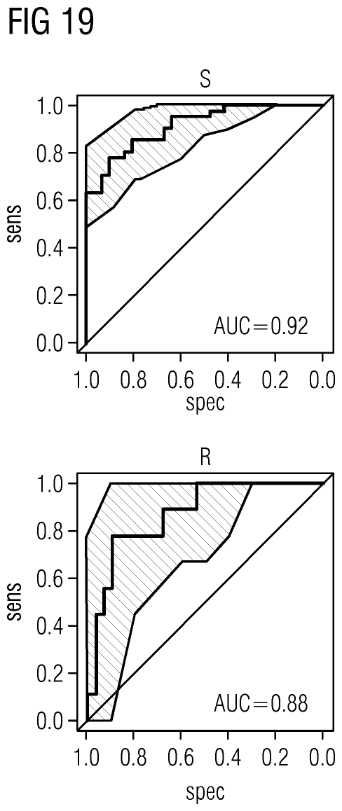

Hence, to search for potential peripheral biomarkers, we investigated if DNA methylation changes are conserved across different tissues. As shown by an exploratory analysis there is indeed a set of conserved directionally-dysmethylated regions in heart tissue and blood, as seen in FIGS. 15 and 16. FIGS. 15 and 16 as well as FIGS. 17 and 18 and 19 show the conservation of DNA methylation signatures across tissues. FIGS. 15 and 16 show an exploratory analysis on the overlap between cardiac tissue and blood DMRs. FIG. 15 particularly shows DCM-associated DMR conserved across tissues for the heart H and the blood B, wherein the relative delta-beta in tissue ≥5%, cardiac tissue (N=41 DCM, N=31 controls), blood (N=41 DCM, N=31 controls). Resulting in the table below are overrepresented gene ontology categories OGOC, particularly contractile fiber part CFP, sarcomere SAR, contractile fiber CF, I band IB, myofibril MF, and Z disc ZD. FIG. 16 particularly shows DCM-associated DMR conserved across tissues for the heart H and the blood B, wherein the relative delta-beta in tissue & blood ≥10%, cardiac tissue (N=41 DCM, N=31 controls), blood (N=41 DCM, N=31 controls). Resulting in the table below are overrepresented gene ontology categories OGOC, particularly hemophilic cell adhesion HCA, cell-cell adhesion via pm CCVP, cell-cell adhesion CCA, biological adhesion BA, calcium ion binding CIB, and cell adhesion CA. Venn diagrams indicating the directional overlap of methylation differences (raw-p≤0.05) in tissue and blood for CpGs with ≥5% or ≥10% relative methylation beta are shown in FIGS. 15 and 16. In the attached tables, overrepresentation analysis on gene ontology categories was performed (FDR-corrected p-values). FIG. 17 depicts the DNA methylation of the NPPA and NPPB locus, particularly for methylation in tissue Meth T (left) and methylation in blood Meth B (right) for each. Natriuretic peptides are the gold-standard biomarkers in HF. In DCM, hypomethylation of the 5′ CpG is associated with increased expression (not shown). In blood, the same direction of dysmethylation is found representing a cross-tissue conservation due to an unknown mechanism. FIGS. 18 and 19 demonstrate that the methylation of cg24884140 is a conserved methylation locus in myocardial tissue and blood. Methylation is shown as methylation beta for tissue Meth beta T on the top and methylation beta for blood Meth beta B at the bottom for screening S and replication R in FIG. 18, whereas FIG. 19 shows a conserved marker panel in blood for screening S at the top and replication R on the bottom, wherein each time sensitivity sense (y-axis) is plotted against specificity spec (x-axis), and the area under the curve AUC is given. Differential methylation is illustrated using nominal p-values. ROC analysis of a DNA methylation signature comprising three CpGs with differential and directed methylation difference in tissue and blood for the detection of DCM/heart failure (B9D1: cg24884140, DCLK2: cg12115081 and NTM: cg25943276).

When using 5% dysmethylation in tissue as a cut-off, we find as many as 3,798 conserved methylation sites that are changed in the same direction in tissue and blood (raw-p≤0.05 in both groups). Very interestingly, the overlapping genes are highly enriched for myofilament components, as seen in the table insets in FIGS. 15 and 16. When further increasing the stringency (10% relative dysmethylation in tissue and blood) 217 conserved methylation sites remain. This is by far higher than expected by chance (p=3.2×10-13), demonstrating a potentially conserved regulation of a relevant number of methylation sites, which further supports the idea to use them as novel biomarkers.

Following this interesting hypothesis, we next explored the epigenetic regulation of the NPPA and NPPB locus. This locus encodes atrial natriuretic factor (ANF) and brain natriuretic peptide (BNP), the latter represents the gold-standard biomarker for heart failure. Astoundingly, we find the same direction of dysmethylation in DNA from heart tissue (FIG. 17, hatched bars top right to bottom left) and peripheral blood (FIG. 17, hatched bars top left to bottom right). As expected, gene expression of NPPA and NPPB is significantly dysregulated in the opposite direction in tissue (upregulation, p=0.0001 for both, data not shown) and transcript levels of NPPB highly correlate with NT-proBNP levels measured in plasma of the patients (R2=0.55). Accordingly, DNA methylation of both loci could already serve as a peripheral biomarker for heart failure.

### Epigenetic Loci as Potential Novel Biomarkers for Heart Failure

In order to embark on the power of connected biological layers captured by the present multistage, multi-omics study design, we then compared the methylation patterns from myocardial tissue and peripheral blood of the screening and replication cohorts after we removed CpG sites that are directly hit by genetic variation (SNP or INDEL within the 50 bp probe region) or are associated with genetic variation within a 10 kb region (α≤0.05). We also removed all CpG sites that have been shown to be associated with blood cell heterogeneity (Holm S. A simple sequentially rejective multiple test procedure. Scandinavian Journal of Statistics. 1979; 6, 65-70). From 90,935 remaining DNA methylation sites, 17,709 were conserved between cardiac tissue and blood, of which 6 (OR=1.38, fisher's exact p=NS) are associated with DCM in heart tissue and 612 (OR=0.89, fisher's exact p=0.01) had disease association in blood. Three epigenetic loci highly significantly overlapped between tissue and blood (OR=28, fisher's exact p<0.001) on all investigated levels, showing disease association and concordant dysmethylation across tissues.

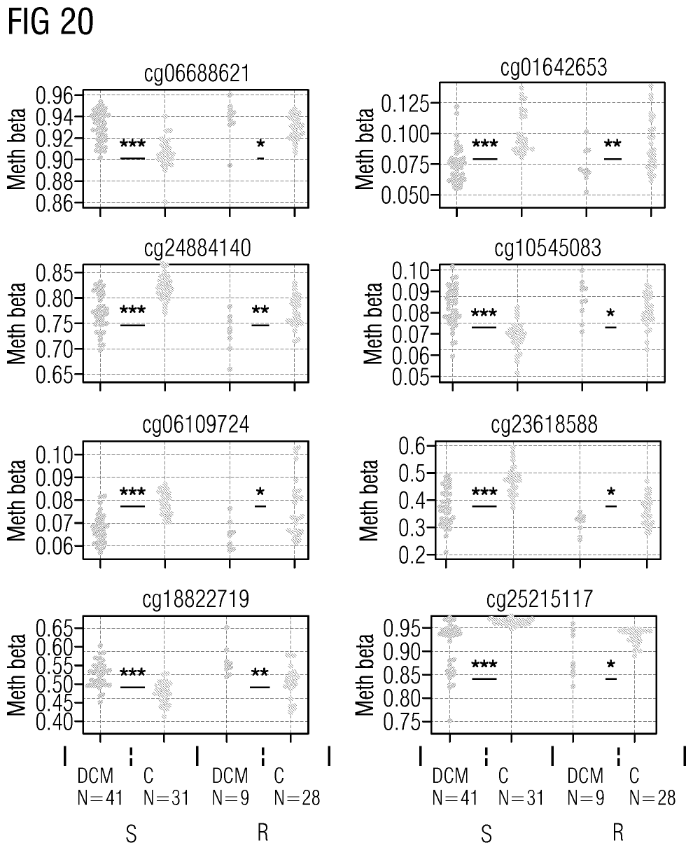

The resolved genes were “B9 Protein Domain 1” (B9D1, hypomethylated in DCM in heart tissue and blood), “Doublecortin like kinase 2” (DCLK2, hypomethylated) and “Neurotrimin” (NTM, hypermethylated). For Neurotrimin (NTM), which belongs to the so-called IgLONS, there is a reported association of its protein blood levels with heart failure and prognosis of affected patients undergoing pharmacotherapy (Cao T H, et al., Identification of novel biomarkers in plasma for prediction of treatment response in patients with heart failure. Lancet. 2015; 385 Suppl 1:S26). B9D1 (cross-validation median p=4.55×10-6), which is also one of the 517 CpGs, as seen in FIG. 14, identified to be robustly associated with DCM in tissue, is one of the most significantly associated hits in blood, as seen in FIG. 20, as well as associated with mRNA transcription in cardiac tissue.

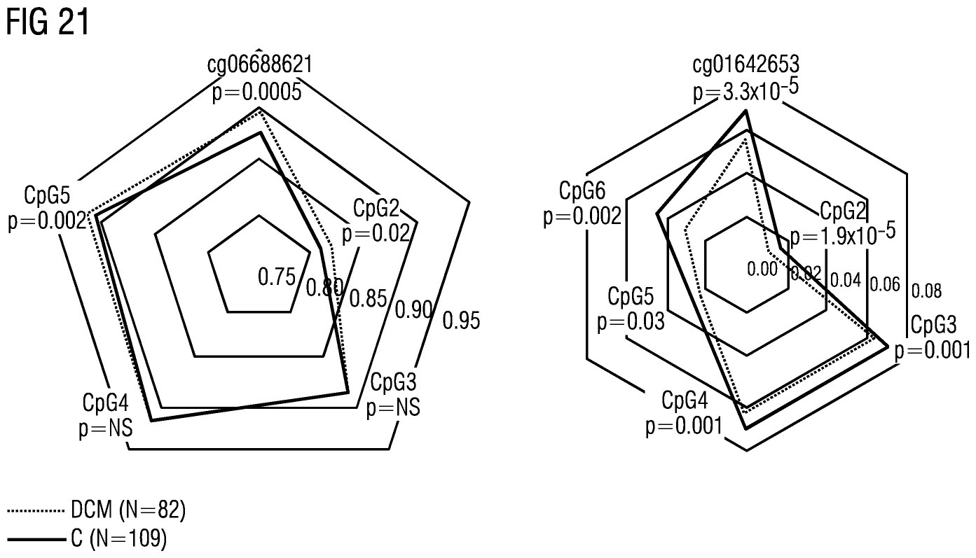

FIGS. 20 and 21 show graphs representing the top 8 individual blood methylation-sites that were verified in the validation cohort. In FIG. 20, the diagram illustrates the verified methylation blood biomarker candidates (*=p≤0.05, **=p≤0.01, ***=p≤0.001), showing DNA methylation in blood for screening S (DCM N=41, controls N=31) and replication R (DCM N=9, controls N=28; replication I), wherein each time methylation beta Meth beta is plotted on the y-axis. While cg06688621 is a DMR in blood only, cg01642653 is dysmethylated in tissue and blood. cg24884140 near B9D1 is also identified by a completely different strategy comprising all assessed levels of multi-omics data. FIG. 21 shows a fine-mapping of the Top-2 marker candidates using mass-spectrometry, particularly showing a finemapping of DNA methalytion in blood (replication II) (DCM N=82, control C N=109). Spider plots show the degree of methylation and significance levels of the lead-CpG and neighboring CpGs for the most significant blood-based DMRs. Dashed line=DCM cases, fat black=healthy controls (NS=not significant).

Mutations in B9D1 result in disturbed heart development due to disrupted cliogenesis and the protein is highly expressed in myocardium and cardiomyocytes (Dowdle W E, et al., Disruption of a ciliary B9 protein complex causes Meckel syndrome. Am J Hum Genet. 2011; 89:94-110). We now show that the methylation state of B9D1 could serve as a diagnostic biomarker for DCM, as exemplified in FIGS. 18 and 19, as we found an AUC of greater 87% in peripheral blood discovery cohort and robust replication in myocardial tissue as well as the peripheral blood verification cohorts. For a 3-marker peripheral blood methylation panel (B9D1: cg24884140, DCLK2: cg12115081 and NTM: cg25943276), we find and AUC of 91.5% in the discovery cohort and 86.9% in the validation cohort, as seen in FIGS. 18 and 19. The single B9D1 DNA methylation as well as the methylation marker panel outperformed NT-proBNP as gold standard marker (AUC of 85%) in this cohort.

Finally, we investigated the DNA dysmethylation sites with highest significance in blood alone and replicated them in the validation cohorts, as seen in FIG. 20. The mean AUC of the best ten markers by this strategy was 0.89 in the screening stage and 0.78 in the replication. The most significant marker with DCM association in blood was cg06688621, which is hypermethylated in DCM. This marker is not differentially methylated in tissue. The second most significant blood marker (raw-p=8.5×10-10) that was successfully replicated is cg01642653 (BDNF, brain-derived neurotrophic factor, which is a cardioprotective factor; Hang P, et al., Brain-derived neurotrophic factor attenuates doxorubicin-induced cardiac dysfunction through activating Akt signalling in rats. J Cell Mol Med. 2017; 21:685-696). This methylation site additionally shows—as other markers in this list—conserved methylation in cardiac tissue (raw-p=9.9×10-4).

By using mass-spectrometry-based DNA methylation quantification as an alternative method in another independent set of 82 DCM cases and 109 controls, as seen in Tables 32 and 33, we were able to fine-map and fully replicate the directional, significant dysmethylation of our Top-2 markers (cg06688621 and cg01642653) and their neighbouring CpGs within the same CpG island.

### Discussion

The present study on the epigenetics of heart failure due to DCM identified a significant role of DNA methylation patterns on cardiac gene transcription in myocardial disease. The reproducible DNA methylation patterns identified in this study as well as the successful replication of previous epigenetic loci from other studies, underline the robustness of the findings and support a role in diagnosis and potentially prognostication of heart failure.

The cardiac epigenome is far from being understood. Basically, only very few studies could reliably map DNA methylation changes in human tissue. While in oncology, the surgical resection of the tumour is integral part of the therapy and hence explanted tissue is readily available for research, the therapy of heart failure does mostly not require surgical intervention and only in rare conditions (e.g. obstructive hypertrophic cardiomyopathy) the resection of myocardium (Kim L K, et al., Hospital Volume Outcomes After Septal Myectomy and Alcohol Septal Ablation for Treatment of Obstructive Hypertrophic Cardiomyopathy: US Nationwide Inpatient Database, 2003-2011. JAMA Cardiol. 2016; 1:324-32). In this study, we were able to refine existing methods for high-quality DNA/RNA extraction and consecutive state-of-the-art sequencing and methylation mapping to assess left-over myocardial tissue from biopsies taken during diagnostics of patients suffering from heart failure due to DCM. By including the largest sample set yet, we were able to detect disease-associated methylation marks at epigenome-wide significance level, replicate them in independent cohorts and show their effect on global cardiac gene expression.

Heart failure is an epidemic threat in industrialized nations. The prevalence is already 37.7 million individuals globally, which comes at total medical costs of more than 20.9 billion $ annually in the US alone (Ziaeian B and Fonarow G C. Epidemiology and aetiology of heart failure. Nat Rev Cardiol. 2016; 13:368-78). To better stratify affected patients or individuals at risk, new molecular biomarkers are desired. By a very systematic approach, we found an intriguing overlap of DNA methylation changes in myocardial tissue and blood. Such an overlap is not expected by chance and the replication of diagnostic statistical performance along with the stringent filtering procedure to avoid confounding from blood cell heterogeneity and genomic variation points to robust epigenetic biomarker patterns. In this early-stage systolic dysfunction cohort, we find methylation markers that outperform NT-proBNP. However, the value of the methylation markers in prognostication, therapy monitoring and decision-making must be rigorously evaluated before concluding any superiority to existing biomarkers.

Applying a very stringent cut-off (5×10-8), five epigenome-wide significant hits were found in this study located on Chr. 1, 3, 14, and 17. When using a lower cut-off for genomewide significance used in other epigenome-wide association (EWA) studies (10-6) (Tsai P C and Bell J T. Power and sample size estimation for epigenome-wide association scans to detect differential DNA methylation. Int J Epidemiol. 2015), as many as 15 loci could be reliably linked to DCM and heart failure. Genes up- or downstream of the five most-stringent methylation marks all show expression in myocardial tissue. While the top hit from the discovery cohort cg16318181 was replicated in the verification cohort, there is no significant interaction between methylation status and expression of the genes within 10,000 bp distance. However, two of the epigenome-wide significant hits showed direct association with mRNA expression levels, namely cg25838968 (gene body region of PLXNA2) and cg16254946 (within the gene body region of GLIS1). PLXNA2 is a member of the Plexin-A family and a receptor for the guiding molecule Semaphorin 3C and has been described in the context of neural crest and cardiac outflow tract development in the sense of GATA6− (Kodo K, et al., GATA6 mutations cause human cardiac outflow tract defects by disrupting semaphorinplexin signaling. Proc Natl Acad Sci USA. 2009; 106:13933-8) and HAND2-related signalling pathways (Morikawa Y and Cserjesi P. Cardiac neural crest expression of Hand2 regulates outflow and second heart field development. Circ Res. 2008; 103:1422-9).

During heart failure pathogenesis, the re-expression of the fetal gene programme is thought to be a central element of initial adaptation to stressors, but ultimately leads to maladaptation and disease progression. The exact mechanisms by which this concerted switch is realized, is unclear. It is known that non-coding RNAs and several promoter elements and transcription factors are involved. In our analysis, we found and replicated DNA methylation changes in the vicinity of several key-regulators of cardiac development. The transcription factor HAND2, for instance, is implicated in cardiomyocyte differentiation and proliferation in the second heart field (McFadden D G, et al., The Hand1 and Hand2 transcription factors regulate expansion of the embryonic cardiac ventricles in a gene dosage-dependent manner. Development. 2005; 132:189-201). During heart failure, Calcineurin/Nfat signalling as well as certain miRNAs (e.g. miR-25) are thought to control HAND2 activation (Dirkx E, et al., Nfat and miR-25 cooperate to reactivate the transcription factor Hand2 in heart failure. Nat Cell Biol. 2013; 15:1282-93).

In our study, we found a change in DNA methylation of the HAND2 locus significantly associated to the regulation of its transcript. IRX5, TBX5, TBX3 and TBX15 and several of their downstream effectors are also altered in the setting of DCM. Altogether 517 CpGs were directionally replicated to be associated with DCM and mRNA transcription. 307 of the 517 were hypomethylated in DCM and 210 were hypermethylated in DCM. The hypomethylated sites correlated with an upregulation of 374 genes and a downregulation of 173 genes corresponding to an upregulation ratio of 2.16. The hypermethylated sites correlated with an upregulation of 204 genes and a downregulation of 171 genes (upregulation ratio of 1.19). Hence, DNA methylation may be involved in the functional reorganisation of important genes during heart failure and these numbers illustrate that the effect of hypomethylation in DCM seems to result mainly in gene (re)activation, while the effect of hypermethylation is balanced (Movassagh M, et al., Distinct epigenomic features in endstage failing human hearts. Circulation. 2011; 124:2411-22).

Only a few regulatory principles have been identified that drive gene expression during development and under pathological conditions in vivo (Sergeeva I A, et al., Identification of a regulatory domain controlling the Nppa-Nppb gene cluster during heart development and stress. Development. 2016; 143:2135-46). Our data indicate that DNA methylation may act alone or in concert with other mechanisms in this context. As an example may serve the NPPA-NPPB gene cluster. NPPA and -B descend from a common ancestral gene by duplication and hence share common chromatin-regulatory mechanisms (Hotel M, et al., HDAC4 controls histone methylation in response to elevated cardiac load. J Clin Invest. 2013; 123:1359-70). Similarly, we found orchestrated hypomethylation of 5′-flanking CpGs of NPPA and NPPB, which is associated with the upregulation of the transcripts atrial natriuretic factor (ANF) and brain natriuretic peptide (BNP). Strikingly, we find the same direction of hypomethylation in peripheral blood, supporting the intriguing finding of conserved heart failure associated DNA methylation patterning across different tissues.

The bimodality of DNA methylation (two copies of homologous DNA) implies a binary on-off control over gene expression, yet a significant number of intermediate methylated loci throughout the genome do not fit within this model (Elliott G, et al., Intermediate DNA methylation is a conserved signature of genome regulation. Nature communications. 2015; 6:6363). To our knowledge, this is the first study that identified a cross-tissue conservation of such epigenetic patterns occurring during heart failure. Due to our cohort and study design, we can exclude that the observed regulation is only due to medication or other confounders. As shown by the example of NPPA/-B, we postulate that heart failure as a syndrome can impose DNA methylation changes due to mechanisms that are sensitive in different cell types representing an epigenomic signature of context-dependent function (Pai A A, et al., A genome-wide study of DNA methylation patterns and gene expression levels in multiple human and chimpanzee tissues. PLoS Genet. 2011; 7:e1001316).

Potential limitations of this study are confounders that influence the epigenetic pattern and DNA methylation. From a technical perspective, we found that genomic variants within the probe region and batch effects are important aspects that need to be considered. To best address this issue, we conducted whole-genome sequencing of patients to identify those sites and measured a random sample of patients multiple times on different arrays on the Infinium platform to define the strata introduced by batches. On the biological level, pharmacotherapy of cases and controls and heterogeneity of tissue are known to be potential confounders, for which we corrected by Principal Component analysis. Using completely independent replication cohorts, we eliminated confounders such as medication of controls, RNA-seq library generation protocols and methylation measurement batch effects. Using mass-spectrometry based DNA methylation measurement, we further substantiated the reliability of our approach for a selection of markers.

The present study provides to our knowledge the most comprehensive mapping of DNA methylation in the human heart and identifies novel loci associated with heart failure and DCM using a comprehensive approach covering genetic variation, DNA methylation and whole transcriptome analyses. To propel epigenetic studies in cardiovascular diseases, it is necessary to develop novel concepts for statistics (power calculation (Tsai P C and Bell J T. Power and sample size estimation for epigenome-wide association scans to detect differential DNA methylation. Int J Epidemiol. 2015), epigenome-wide significance levels, differential methylation models (Wang S. Method to detect differentially methylated loci with case-control designs using Illumina arrays. Genet Epidemiol. 2011; 35:686-94)), appropriate study designs incorporating different biological levels (multi-omics) and definition of adequate controls and confounders. Especially for myocardial tissue, lack of healthy controls constrains the elucidation of cardiac epigenetics. In the present study, we compared failing myocardium against non-failing tissue derived from transplanted hearts showing regular function and a smaller control group of donors that suffered road accidents. Importantly, we show that it is worth studying DNA methylation in peripheral blood, for which adequate controls are often available.

It will be interesting to systematically evaluate DNA methylation markers in longitudinal cohorts of heart failure due to different etiologies including ischemic heart disease. The potential indication of the here detected methylation markers point towards earlier detection of systolic dysfunction and heart failure, but they could also be evaluated for therapy selection and monitoring.

The presently described method allows an efficient and improved tool for finding markers in patients, particularly for non-infectious diseases, like HF and DCM.

With the presently found markers, an improved, early detection and prognosis of HF/DCM, patient stratification for therapy decision support, and optimized, personalized treatment is possible.

This invention reports molecular markers which are indicative of HF/DCM or of the risk developing HF/DCM or for a prediction of therapy effects or therapy outcome.

The present study provides to the knowledge of the inventors the first epigenome-wide association study in living patients with heart failure using a multi-omics approach.

1. A method of determining markers for a disease from a patient, comprising
obtaining or providing at least one sample of peripheral blood and at least one sample of a diseased tissue of the patient diagnosed with the disease;
obtain an epigenomics profile and/or analyze a transcriptome of the at least one sample of the peripheral blood and the at least one sample of the diseased tissue;
compare the epigenomics profile and/or the transcriptome to an epigenomics profile and/or a transcriptome of a suitable control, respectively; and
determine one or more alteration in the epigenomics profile and/or the transcriptome in both the at least one sample of the peripheral blood and at least one sample of the diseased tissue of the patient diagnosed with the disease.

2. A method of determining markers for a disease from a patient, comprising
obtaining or providing at least one sample of peripheral blood or at least one sample of the diseased tissue of the patient diagnosed with the disease;
obtain an epigenomics profile and analyze a transcriptome of the at least one sample of the peripheral blood and at least one sample of the diseased tissue;
compare the epigenomics profile and the transcriptome to an epigenomics profile and a transcriptome of a suitable control, respectively; and
determine one or more alteration in the epigenomics profile and the transcriptome in either at least one sample of the peripheral blood or the at least one sample of the diseased tissue of the patient diagnosed with the disease.

3. The method of claim 1, wherein the patient is a human.

4. The method of claim 1, wherein the disease is heart failure (HF) and/or dilated cardiomyopathy (DCM).

5. The method of claim 4, wherein the sample of the diseased tissue is obtained from myocardial tissue.

6. The method of claim 1, wherein the alteration is a hyper and/or hypo methylation and/or a change in the RNA expression level

7. The method of claim 1, wherein a plurality of samples of the peripheral blood and/or the diseased tissue are obtained or provided from patients diagnosed with the disease.

8. A method of determining a risk for a disease in a patient, comprising
obtaining or providing an epigenomics profile and/or a transcriptome of at least one sample of the peripheral blood and/or a diseased tissue of the patient, and
determining the presence of at least one marker as determined by the method of claim 1.

9. The method of claim 8, wherein the diseased tissue is the myocard and the disease is heart failure and/or dilated cardiomyopathy.

10. The method of claim 9, wherein the at least one epigenetic and/or transcriptomic marker
is contained in genomic regions with regard to reference genome hg19 that show coordinated hyper/hypo methylation in HF/DCM in peripheral blood and myocardial tissue and are associated with RNA expression levels and is chosen from the sequences disclosed in Table 1; and/or
is contained in genomic regions with regard to reference genome hg19 that show hyper/hypo methylation in HF/DCM in myocardial tissue and are associated with RNA expression levels and is chosen from the sequences disclosed in Table 2; and/or
is contained in genomic regions with regard to reference genome hg19 that show coordinated hyper/hypo methylation in HF/DCM in peripheral blood and myocardial tissue and is chosen from the sequences disclosed in Table 3; and/or
is contained in genomic regions with regard to reference genome hg19 that show dysmethylation in HF/DCM in peripheral blood and is chosen from the sequences disclosed in Table 4; and/or
is contained in genomic regions with regard to the reference Infinium HumanMethylation450K database and the reference genome hg19, respectively, that show dysmethylation in HF/DCM in peripheral blood and is chosen from the cpg IDs or positions disclosed in Table 5; and/or
is contained in genomic regions with regard to reference genome hg19 that show dysmethylation in HF/DCM in peripheral blood and is chosen from the sequences disclosed in Table 6; and/or
is contained in genomic regions with regard to the reference Infinium HumanMethylation450K database and the reference genome hg19, respectively, that show dysmethylation in HF/DCM in peripheral blood and is chosen from the cpg IDs or positions disclosed in Table 7; and/or
is contained in genomic regions with regard to reference genome hg19 that show dysmethylation in HF/DCM in peripheral blood and is chosen from the sequences disclosed in Table 8; and/or
is contained in genomic regions with regard to the reference Infinium HumanMethylation450K database and the reference genome hg19, respectively, that show dysmethylation in HF/DCM in peripheral blood and is chosen from the cpg IDs or positions disclosed in Table 9; and/or
is contained in genomic regions with regard to reference genome hg19 that show dysmethylation in HF/DCM in peripheral blood and myocardial tissue and are associated with RNA expression levels and is chosen from the ANF and/or BNP loci and/or the sequences disclosed in Table 10.

11. The method of claim 10, wherein presence of a plurality of markers is determined.

12. (canceled)

13. A data bank comprising the markers disclosed in claim 10.

14. A method of determining a risk for a disease in a patient, comprising
obtaining or providing data of an epigenomics profile and/or a transcriptome of at least one sample of the peripheral blood and/or a diseased tissue of the patient, and
determining the presence of at least one marker as determined by the method of claim 1.

15. A computer program product comprising computer executable instructions which, when executed, perform a method according to claim 14.

16. A method of prognosis and/or for monitoring and/or assisting in drug-based therapy of a patients diagnosed with heart failure and/or dilated cardiomyopathy, the method comprising determining the presence of at least one marker of claim 10 in the patient.
